(19) United States
(12) Patent Application Publication
(10) Pub. No.: US 2024/0327525 A1
(43) Pub. Date: Oct. 3, 2024

(54) Cation-Independent Mannose-6-Phosphate Receptor Binders For Targeted Protein Degradation

(71) Applicants: VIB VZW, Gent (BE); Universiteit Gent, Gent (BE)

(72) Inventors: Nico Callewaert, Nevele (BE); Justine Naessens, Gent (BE); Linde Van Landuyt, BELGIUM (BE)

(21) Appl. No.: 18/293,467

(22) PCT Filed: Jul. 29, 2022
(86) PCT No.: PCT/EP2022/071381

§ 371 Date: Jan. 30, 2024

(30) Foreign Application Priority Data
Jul. 30, 2021 (EP) 21188724.5

(51) Int. Cl. C07K 16/28; A61K 39/00; C07K 16/46

(52) U.S. Cl. CPC C07K 16/2863; C07K 16/468; A61K 2039/505; C07K 2317/24; C07K 2317/31; C07K 2317/34; C07K 2317/565; C07K 2317/569; C07K 2317/77; C07K 2317/92

(57) ABSTRACT

The present invention relates to protein binding agents specifically binding the human cation-independent mannose-6-phosphate receptor (CI-M6PR), more specifically polypeptide agents comprising an immunoglobulin single variable domain (ISVD) specifically binding CI-M6PR at nano- to picomolar affinity, fused to further protein binding agents specifically binding extracellularly-accessible protein targets, such as membrane proteins, extracellular or secreted proteins. More specifically said CI-M6PR-specific ISVD recognizes CI-M6PR N-terminal domains 1, 2 and/or 3, thereby providing for means and methods for internalization, lysosomal targeting and degradation of agents comprising said ISVD, and of targets bound to said protein binding agents. The CI-M6PR binders disclosed herein are thus linked or fused to a further protein binding agent, in particular another antigen-binding protein, such as an ISVD or antibody, relevant for use in therapy, more specifically for treatment of diseases affected by said target antigen bound by said antigen-binding protein. More specifically disclosed herein are CI-M6PR ISVD fusions to antigen-binding proteins specifically binding EGFR, for treatment of cancer.

### CROSS-REFERENCE TO RELATED APPLICATIONS

This application is a national phase entry under 35 U.S.C. § 371 of International Patent Application PCT/EP2022/071381, filed Jul. 29, 2022, designating the United States of America and published in English as International Patent Publication WO 2023/016828 on Feb. 16, 2023, which claims the benefit under Article 8 of the Patent Cooperation Treaty to European Patent Application Serial No. 21188724.5, filed Jul. 30, 2021, the entireties of which are hereby incorporated by reference.

### INCORPORATION BY REFERENCE

The ST.26 XML Sequence listing named “10337USSequenceListing”, created on Aug. 1, 2022, and having a size of 114,744 bytes, is hereby incorporated herein by this reference in its entirety.

### FIELD OF THE INVENTION

The present invention relates to protein binding agents specifically binding the human cation-independent mannose-6-phosphate receptor (CI-M6PR), more specifically polypeptide agents comprising an immunoglobulin single variable domain (ISVD) specifically binding CI-M6PR at nano- to picomolar affinity, fused to further protein binding agents specifically binding extracellularly-accessible protein targets, such as membrane proteins, extracellular or secreted proteins. More specifically said CI-M6PR-specific ISVD recognizes CI-M6PR N-terminal domains 1, 2 and/or 3, thereby providing for means and methods for internalization, lysosomal targeting and degradation of agents comprising said ISVD, and of targets bound to said protein binding agents. The CI-M6PR binders disclosed herein are thus linked or fused to a further protein binding agent, in particular another antigen-binding protein, such as an ISVD or antibody, relevant for use in therapy, more specifically for treatment of diseases affected by said target antigen bound by said antigen-binding protein. More specifically disclosed herein are CI-M6PR ISVD fusions to antigen-binding proteins specifically binding EGFR, for treatment of cancer.

### BACKGROUND

Small-molecule drugs act by binding to a well-defined pocket of a disease-causing protein and modulating its function. However, as many proteins miss such crevices, as much as 85% of the human proteome is currently considered to be undruggable (Neklesa, et al., 2017, Pharmacol. Ther. 174, 138-144). This has been challenged with the emergence of PROteolysis TArgeting Chimera (PROTAC) technology, a therapeutic modality that exploits the ubiquitin-proteasome system for selective degradation of an intracellular target protein (Sakamoto, et al. (2001) Proc. Natl. Acad. Sci. 98, 8554-8559). Such a degrader consists of a binder of an E3 ligase coupled to a ligand that can bind to any site of the target protein. Besides enabling the targeting of undruggable proteins, the targeted protein degradation strategy has another advantage over inhibition-based treatment strategies: the removal of a protein ablates all of its functions, which is important for example when the protein acts as a signal transduction scaffold. However, as PROTACs make use of the cytosolic protein degradation machinery, they are inherently limited to target engagement within the intracellular environment.

Nanobodies are the variable domains of camelid-derived heavy chain-only antibodies (VHHs), that are characterized by their small size (+15 kDa) [1]. This enables good tissue-penetration, while maintaining similar potency and binding specificity as of conventional antibodies [3]. As modular building blocks, VHHs can be easily concatenated in multivalent and/or multispecific formats, which is exploited in this approach. As VHHs are also highly stable and soluble, they can be easily and cost-effectively manufactured in lower organisms such as bacteria and yeast [4]. Among the explored intracellular TPD approaches, Nanobody-based fusions referred to as the ARMeD system, have been shown to provide for a Nb specifically targeting a protein of interest, coupled to the RING domain of the E3 ubiquitin ligase RNF4, thereby triggering degradation without off-target effects upon delivery into the cell (Zhong et al. Eur J Med Chem. 2022; 231:114142; Ibrahim, et al. Molecular Cell, 2020. 79, (1), 155-166.e9).

Due to their great conformational stability, they possess a high intrinsic pH and protease resistance [1], which are attractive properties for cycling through the endosomal-lysosomal system. Furthermore, VHH-based formats are suitable for various routes of administration, including via intravenous injection and inhalation, positioning them as ideal components for therapeutic purposes. GlueTACs for instance are covalent antigen-binding Nanobody-based chimera targeting a membrane protein and conjugated to cell-penetrating peptide and lysosomal sorting sequence for triggering lysosomal degradation (Zhang, et al. J. American Chem. Society. 2021. 143 (40), 16377-16382).

Indeed, lysosomes are acidified organelles of the cells containing more than 70 hydrolytic enzymes. These enzymes are responsible for the degradation of cleavable cellular macromolecules to their original building blocks [2]. Macromolecules generally reach the lysosome via endocytosis, phagocytosis or endocytosis after which each elementary unit can be recycled and used for the synthesis of other macromolecules or can be further metabolized as a supply for energy.

Membrane-bound protein targets are known to be ubiquitinated through expression of membrane-bound E3 ligases, thereby inducing their endocytosis and lysosomal degradation. Novel technologies have demonstrated making use of this mechanism to apply membrane-bound E3 ligases for co-targeting membrane or extracellular proteins for degradation. For instance, AbTACs as reported by Cotton et al. (J. Am. Chem. Soc. 2021, 143, 593-598); and the heterobifunctional molecules targeting membrane-bound E3 ligases and transmembrane target proteins as reported by Maurice (WO2021/176034A1).

Degradation of extracellularly-accessible proteins may also be enabled by exploiting the lysosome, from the outside through receptor-mediated endocytosis via the cation-independent mannose-6-phosphate receptor (CI-M6PR), a P-type lectins on the cell's plasma membrane, which constantly recycles through the endolysosomal pathway, and thereby efficiently internalizing and delivering proteins or targets bound to the receptor into endosomes and lysosomes. So, a further application is based on the acidic pH in the endosomes, which results in dissociation of a cargo or complex from the CI-M6PR receptor at a pH around 5.8 in a late endosomal stage [20], and allows rapid recycling of the CI-M6PR receptor itself, which constantly shuttles between the cell surface and the late-endosomal compartments in virtually all cell types and is able to target extracellular ligands to the lysosome (Dahms, et al. (1989), S. J. Biol. Chem. 264, 12115-12118).

Thus, CI-M6PR cargos are efficiently delivered to lysosomes through the endocytotic cycle, a concept that is used in design of lysosome-targeting chimaeras (LYTACs) [10], in analogy with PROTACs, providing for an alternative format that couples a complex chemically-synthetized glycopeptide ligand of the CI-M6PR to an anti-target antibody. LYTACS were shown to enable the depletion of secreted and membrane-associated proteins and as agonists of the CI-M6PR [10]. LYTACs were shown to in vitro internalize and degrade a selection of both extracellular and transmembrane proteins when administered to cells. However, a downside for in vivo applications and in terms of large-scale production is the large size of the construct (+150 kDa of monoclonal antibodies), which can hinder its biodistribution in solid tissues [21], and for which recombinant expression in mammalian cells is required. Moreover, when mannose-6-phosphonate (M6Pn) glycopolypeptides are used for binding the CI-M6PR, the long synthesis process to produce the ligand and subsequent conjugation to the antibody is highly complex and very expensive. Indeed, the production of the mannose-6-phosphonate (M6Pn) glycopolypeptide ligand and subsequent conjugation to the antibody involves a 13-step synthesis process.

An interesting example of an extracellularly-accessible protein target is for instance the human epidermal growth factor (EGFR), which is a transmembrane receptor tyrosine kinase (RTK) that plays a central role in the growth and maintenance of epithelial tissues. It is frequently overexpressed and drives disease progression in many types of cancer, including an estimated 60-80% of colorectal cancers (CRCs) [23]. Chemotherapy is usually the first-line treatment for irresectable metastatic CRC (mCRC) and in patients with RAS-wild type (WT) cancers, this is combined with one of the two approved anti-EGFR monoclonal antibodies (mAbs) Cetuximab or Panitumumab. These mainly exert their function through the antagonization of EGF-stimulating activation of EGFR, inhibiting its kinase function as a consequence. The addition of such mAbs to chemotherapy for the treatment of RAS-WT mCRCs has shown increased overall survival of several months as compared to chemotherapy alone [24]. However, activating mutations in KRAS (the predominantly mutated RAS isoform in CRC), which is a downstream component of an EGFR signaling pathway, occur in approximately 35-45% of CRCs and are the main intrinsic resistance mechanisms to anti-EGFR mAbs [25]. But even among the RAS-WT mCRCs, those harbouring a V4600E mutation in the BRAF gene also fail to respond to treatment [26]. Furthermore, acquired resistance to anti-EGFR mAbs arises in virtually all patients, in half of cases caused by secondary mutations in the KRAS gene and sometimes in the EGFR extracellular domain, escaping antibody binding [28]. In this context, targeted degradation of the EGFR may offer an exciting new strategy to overcome intrinsic and acquired resistance. It has been shown that EGFR downregulation, as opposed to EGFR inhibition, induces cell death in a range of cancer cells, including the KRAS-mutated HCT116 cell line that has a relatively low EGFR expression and does not respond to Cetuximab [29]. Indeed, kinase-inhibited EGFR can function as a scaffolding node for interaction with survival proteins and maintenance of downstream pro-survival signaling in several ways [29-30].

So for numerous therapeutic applications a lysosomal targeting approach as an aid in targeted protein degradation would be beneficial as an alternative mechanism of action to provide for a novel medical modality. So, there is a need to generate next-generation lysosome targeting binding agents, which overcome the above hurdles of the existing lysosomal targeting strategies.

### SUMMARY OF THE INVENTION

With the purpose of providing for a new type of binding agent capable of mediating lysosomal targeting through reversible binding to the CI-M6PR receptor, the present invention is based on the application of human to mouse cross-reactive immunoglobulin single variable domains (ISVDs), in particular VHHs, that bind the CI-M6PR at physiological pH and dissociate from it in a pH-dependent manner, resulting in lysosomal uptake (Callewaert et al., PCT/EP2022/054278). A covalent coupling of such anti-CI-M6PR VHH to a further binder specific for an extracellular, secreted, or transmembrane target protein eventually results in a novel modality for CI-M6PR-mediated lysosomal uptake and degradation. So the present invention relates to a new VHH-based LYTAC-format, also called nanoLYTAC, wherein the efficacy and potency of the endosomal/lysosomal targeting on the one hand relies on the properties of the fusion protein provided by the immunoglobulin single variable domain (ISVD) that recognizes the CI-M6PR for recycling, and on the other hand, on the coupled binding agent specific for the extracellularly-accessible target protein. It was found that this new format provides for a number of substantial benefits over the existing extracellular targeted protein degradation modalities.

By using specifically designed and characterized anti-CI-M6PR VHHs for lysosomal targeting, the alternative Nanobody-based LYTACs (or nanoLYTACs) form a functional bispecific therapeutic tool to deliver other, coupled, binding agents, preferably also comprising an antigen-binding protein domain, such as an antibody, or more specifically an ISVD or VHH, for lysosomal degradation, wherein said binding agents in their turn can be selected for their properties in targeting certain extracellularly-accessible proteins of interest. As a proof of concept, the characterized VHHs specific for CI-M6PR as reported in Callewaert et al. (PCT/EP2022/054278) were coupled to antigen-binding proteins known to target EGFR, a transmembrane receptor, as exemplified herein. Further POC was evidenced showing that endocytotic internalization and/or lysosomal degradation, was obtained, based on the coupling with at least two types of the CI-M6PR-specific VHHs disclosed herein, wherein each type is characterized to bind to a CI-M6PR epitope located in the N-terminal domains 1-3, as characterized in Callewaert et al. (PCT/EP2022/054278). Indeed, said panel of VHHs has previously been characterized as a panel of CI-M6PR binders with different pH dependencies for their association with the receptor, therefore resulting in a toolbox that is useful in designing the customized Nb-based LYTACs in view of the desired outcome or treatment purposes.

The present invention relates to multi-specific lysosome targetable anti-CI-M6PR binding agents, called nano-lysosomal targeting chimeras or nanoLYTACs, and is based on the identification of a panel of VHHs that specifically bind to human and mouse CI-M6PR its N-terminal region, present on the extracellular side of the plasma membrane, thereby enabling traffic through the endolysosomal pathway. Moreover, the anti-CI-M6PR VHHs adopt specific pH-dependent dissociation properties, which promote delivery to the lysosomal compartment. Fusions of these anti-CI-M6PR VHHs moieties to further protein binding agents, preferably involving antigen-binding for targeting other extracellular or membranous targets, enables to apply these binding agents in targeted lysosomal internalization and protein target degradation.

A first aspect of the invention thus relates to protein binders containing an immunoglobulin-single-variable domain (ISVD) which specifically bind human cation-independent mannose-6-phosphate receptor (CI-M6PR; also known as IGF2R), specifically recognizing a binding site located on the extracellular N-terminal domains 1, 2 and/or 3 of human CI-M6PR, and wherein said ISVD is fused to a protein binding domain or agent specifically binding an extracellularly-accessible target. More specifically, said CI-M6PR-specific ISVD of said protein binding agent provides for a high affinity binding to the receptor, in vitro or in cells, with a K_{D} value in the range of 100 nM or lower. More specifically, said protein binding agent internalizes in the cells upon binding to the CI-M6P Receptor. Preferably, said protein binding agent upon binding to the CI-M6PR internalizes in the cell as a complex with the extracellularly-accessible target bound to the coupled binding agent specifically binding said extracellularly-accessible target.

In a specific embodiment, said protein binding agent (also referred to herein as nanoLYTAC) comprises an ISVD specifically binding CI-M6PR, which specifically recognizes a binding site positioned on N-terminal domains 2 and 3, and is defined by the epitope comprising the amino acid residues 191, 194-197, 208, 219, 224, 225, 297, 357,408-409, 431, 433 and 457 as depicted in SEQ ID NO:23. A further specific embodiment provides for said binding agent comprising an ISVD which specifically binds through interaction of its residues 32, 52-57, 100-103, and 108 as set forth in SEQ ID NO:8, with the residues depicted herein as epitope in the N-terminal domains 2 and 3 of CI-M6PR.

Another specific embodiment relates to said protein binding agent (also referred to herein as nanoLYTAC) comprising an ISVD specifically binding CI-M6PR, which specifically recognizes a binding site positioned on N-terminal domain 1, and is defined by the epitope comprising the amino acid residues 59, 60, 85, 87, 89, 146, 147, and 148 and 118 or 119 as set forth in SEQ ID NO:23. A further specific embodiment provides for said binding agent comprising an ISVD which specifically binds through interaction of its residues 31, 33, 35, 53, 54, 56, 57, 96, and 104, as set forth in SEQ ID NO:7, or the residues 31-35, 50, 52-57, 96-98 as set forth in SEQ ID NO:24, with the residues depicted herein as epitope in the N-terminal domain 1 of CI-M6PR.

In a specific embodiment, said binding agent comprises or consists of a fusion protein comprising a CI-M6PR-specific ISVD as described herein, and a binder specifically binding an extracellularly-accessible protein target, which are fused directly or via a linker, and preferably wherein said ISVD is structured according to the following formula (1): FR1-CDR1-FR2-CDR2-FR3-CDR3-FR4 (1), and comprising the CDR1, CDR2 and CDR3 regions as selected from the CDR1, CDR2 and CDR3 regions of an ISVD sequence selected from the group of SEQ ID NO: 1, 5, 7, 8, 24 or 25, wherein the CDR regions are annotated according to Kabat, MacCallum, IMGT, AbM, or Chothia. In a specific embodiment, said M6PR-specific ISVDs comprise CDR1, CDR2, and CDR3 from SEQ ID NO:1, or CDR1, CDR2, and CDR3 from SEQ ID NO:5, or CDR1, CDR2, and CDR3 from SEQ ID NO:7, or CDR1, CDR2, and CDR3 from SEQ ID NO:8, or CDR1, CDR2, and CDR3 from SEQ ID NO:24, or CDR1, CDR2, and CDR3 from SEQ ID NO: 25, wherein said CDRs may be defined according to the annotation of Kabat, MacCallum, IMGT, AbM, or Chothia, as further defined herein.

A further embodiment relates to said protein binding agent described herein, wherein the CI-M6PR-specific ISVD comprises a CDR1 sequence selected from SEQ ID NO:36-41, a CDR2 sequence selected from SEQ ID NO:42-47, and a CDR3 sequence selected from SEQ ID NO:48-53, or alternatively comprises an ISVD with:
CDR1 consisting of SEQ ID NO:36, CDR2 consisting of SEQ ID NO:42, and CDR3 consisting of SEQ ID NO:48,
CDR1 consisting of SEQ ID NO:37, CDR2 consisting of SEQ ID NO:43, and CDR3 consisting of SEQ ID NO:49,
CDR1 consisting of SEQ ID NO:38, CDR2 consisting of SEQ ID NO:44, and CDR3 consisting of SEQ ID NO:50,
CDR1 consisting of SEQ ID NO:39, CDR2 consisting of SEQ ID NO:45, and CDR3 consisting of SEQ ID NO:51,
CDR1 consisting of SEQ ID NO:40, CDR2 consisting of SEQ ID NO:46, and CDR3 consisting of SEQ ID NO:52, or
CDR1 consisting of SEQ ID NO:41, CDR2 consisting of SEQ ID NO:47, and CDR3 consisting of SEQ ID NO:53.

A further embodiment relates to said protein binding agent comprising a CI-M6PR-specific ISVD comprising said CDRs of SEQ ID NO: 1, 5, 7, 8, 24 or 25, annotated according to AbM, and comprising a FR1 sequence corresponding to SEQ ID NO:78, a FR2 sequence corresponding to SEQ ID NO:79, a FR3 sequence corresponding to SEQ ID NO: 80, and a FR4 sequence corresponding to SEQ ID NO: 81, or alternatively a FR1 sequence selected from SEQ ID NO:54-59, FR2 sequence selected from SEQ ID NO:60-65, FR3 sequence selected from SEQ ID NO:66-71, and FR4 sequence selected from SEQ ID NO:72-77, or alternatively comprising:
FR1 consisting of SEQ ID NO:54, FR2 consisting of SEQ ID NO:60, FR3 consisting of SEQ ID NO: 66, and FR4 consisting of SEQ ID NO: 72,
FR1 consisting of SEQ ID NO:55, FR2 consisting of SEQ ID NO:61, FR3 consisting of SEQ ID NO: 67, and FR4 consisting of SEQ ID NO: 73,
FR1 consisting of SEQ ID NO:56, FR2 consisting of SEQ ID NO:62, FR3 consisting of SEQ ID NO: 68, and FR4 consisting of SEQ ID NO: 74,
FR1 consisting of SEQ ID NO:57, FR2 consisting of SEQ ID NO:63, FR3 consisting of SEQ ID NO: 69, and FR4 consisting of SEQ ID NO: 75,
FR1 consisting of SEQ ID NO:58, FR2 consisting of SEQ ID NO:64, FR3 consisting of SEQ ID NO: 70, and FR4 consisting of SEQ ID NO: 76, or
FR1 consisting of SEQ ID NO:59, FR2 consisting of SEQ ID NO:65, FR3 consisting of SEQ ID NO: 71, and FR4 consisting of SEQ ID NO: 77,
or a humanized variant of any thereof, as further described herein.

Another embodiment relates to said binding agents wherein said CI-M6PR-specific ISVD comprises a sequence selected from the group of sequences of SEQ ID NO:1, 5, 7, 8, 24 or 25, or a sequence with at least 85% amino acid identity thereof, containing identical CDRs as in SEQ ID NO: 5, 7, 8, 24 or 25, or a humanized variant thereof, as defined further herein, or such as presented in SEQ ID NOs: 26-35.

A further specific embodiment relates to the binding agent as described herein which is a multi-specific or multivalent binding agent. More particularly bivalent or bispecific agents are envisaged herein. Even more specific, a multi-specific protein binding agent is envisaged, comprising an ISVD which specifically binding human CI-M6PR, specifically recognizing a binding site located on the extracellular N-terminal domains 1, 2 and/or 3 of human CI-M6PR, as defined herein, and fused or linked to a binding agent specifically binding an extracellularly-accessible target, wherein said fusion or linking is made by a direct coupling or via a linker, which may be a short peptide linker, or a polypeptide moiety such as an Fc-tail or another moiety, which may comprise a further antigen-binding domain or more specifically an ISVD. Specifically, said binding agent comprising an ISVD specifically binding CI-M6PR, may comprise a binding moiety specifically binding a cell surface or extracellular molecule, which y specifically also comprises an ISVD for binding the extracellularly-accessible target, and/or a further moiety.

In a specific embodiment, said fusion protein or binding agent of the invention is a multispecific fusion protein, comprising the CI-M6PR-specific ISVD of the present invention, fused to a protein binder specifically binding an extracellular-accessible target, and optionally a further moiety, of which any of said components may be labelled for detection, or may provide for a tag or label.

Another embodiment relates to said protein binding agent of the invention comprising a multispecific fusion protein, comprising the CI-M6PR-specific ISVD of the present invention, fused to a protein binder specifically binding an extracellular-accessible target, and optionally a further moiety, wherein said target-specific protein binder comprises or consists of an antigen-binding protein domain, more specifically comprises an ISVD, or an antibody or active fragment thereof, or specifically an IgG, or any type of VHH-Fc fusion format. In a further specific embodiment, said further moiety is a functional moiety, preferably comprising an antigen-binding domain, such as a therapeutic moiety, which preferably binds a further target, and/or a half-life extension.

In a specific embodiment disclosed herein, said protein binding agent of the present invention comprises a binding agent specifically binding the transmembrane protein Epidermal growth factor receptor (EGFR) at the extracellular site. More specifically said fusion protein comprises a CI-M6PR specific ISVD as described herein, and an EGFR-specific binding agent comprising an ISVD consisting of SEQ ID NO:12, 17, or a homologue with at least 90% identity thereof wherein the CDRs are identical, or comprising an antibody composed of the heavy chain as shown in SEQ ID NO: 87, and the light chain as shown in SEQ ID NO: 86 to provide a EGFR-specific conventional antibody binding as EGFR-specific binder, more specifically said protein binding agent may comprise SEQ ID NO: 88 or 89, and SEQ ID NO: 86. Alternatively, the Protein binding agent of the present invention, specifically binding the extracellularly-accessible protein target EGFR comprises a sequence selected from the group of sequences of SEQ ID NOs: 13, 14, 18, 19, 82 to 85, or a functional homologue with at least 90% identity thereof wherein the CDRs are identical, or the heavy chain-VHH fusion of SEQ ID NO: 88 or 89 provided as EGFR-specific antibody with the light chain SEQ ID NO:86.

Another aspect relates to a nucleic acid encoding the protein binding agent or fusion protein comprising a CI-M6PR-specific ISVD fused to the extracellularly-accessible target-specific protein binding agent, as described herein, or the further combined multi-specific binding agents. Furthermore, a vector comprising said nucleic acid molecule, for expression of said binding agents or fusion proteins is disclosed herein.

Another aspect relates to the application or use of the binding agent, the multi-specific binding agent, the fusion protein, or the nucleic acid disclosed herein, in drug discovery, in structural analysis, or in a screening assay, such as for instance in structure-based drug discovery or fragment-based screening assay.

Another aspect relates to production methods for obtaining the binding agent as described herein, comprising the steps of providing a fusion protein of the present invention by recombinant expression of the nucleic acid molecule, and optionally a further nucleic acid molecule (in the case of antibody expression), in a host, and purification of the fusion protein, optionally in the format of a antibody formed by the fusion protein and further antibody chain, from said host.

Further embodiments relate to the application or use a multi-specific binding as described herein, for instance a bispecific agent, comprising an ISVD specifically binding CI-M6PR and a second antigen binding domain for binding an extracellularly-accessible target protein, in a method for degrading said target, which is a cell surface molecule or extracellular molecule or transmembrane protein, through lysosomal uptake of said multispecific agent in the lysosome, when bound to said target. A specific embodiment further discloses the use of said binding agent, multi-specific binding agent or fusion protein as described herein for in vitro lysosomal tracking, optionally when operably linked or chemically coupled to a label.

A further aspect relates to a pharmaceutical composition comprising any of the binding agents described herein, multi-specific binding agents, or fusion proteins described herein.

Another aspect of the invention relates to the medical use of the binding agent, the multi-specific binding agent, the fusion protein, or the pharmaceutical composition as described herein. More specifically said agents or proteins for use in treatment of a lysosomal storage disease, or for use in Enzyme-replacement therapy. Another embodiment of the invention relates to the multi-specific binding agent, or the pharmaceutical composition comprising said multispecific binding agent, as described herein, for use in a disorder related to the target of the disease caused by or related to the extracellularly accessible protein target, specifically bound said binding agent, more specifically, a target which is a cell surface or extracellular molecule. Specifically, in one embodiment said target is EGFR, providing for a binding agent for use in treatment of cancer.

A final aspect of the invention relates to said binding agent, multi-specific binding agent, fusion protein, or labelled form thereof, for use as a diagnostic or for in vivo imaging.

### DESCRIPTION OF THE FIGURES

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

FIG. 1. SDS-PAGE analysis of LYTAC expression tests in Pichia pastoris. Constructs 14-19 (composition indicated in Table 1) were produced in wild type Pichia pastoris (i.e. NCYC2543) and 20 μl of supernatant was analyzed on SDS-PAGE. ‘MM’=molecular weight marker (Precision Plus Protein Standard, Bio-Rad)

FIG. 2. SDS-PAGE analysis of endoglycosidase H (EndoH)-digest of LYTACs. Constructs 14-19 (composition indicated in Table 1) were produced in wild type Pichia pastoris (i.e. NCYC2543) and 19 μl of the supernatant was incubated at 37° C. overnight with EndoH, after which it was analyzed on SDS-PAGE. ‘MM’=molecular weight marker (Precision Plus Protein Standard, Bio-Rad)

FIG. 3. SDS-PAGE analysis of LYTAC expression tests in Pichia pastoris. Constructs 26-29 (composition indicated in Table 1) were produced in WT Pichia pastoris and 20 μl of supernatant was analyzed on SDS-PAGE. The clone indicated in red was selected for larger-scale expression and purification. ‘MM’=molecular weight marker (Precision Plus Protein Standard, Bio-Rad)

FIG. 4. SDS-PAGE of gravity flow Immobilized Metal Ion Chromatography (IMAC) purification on LYTAC constructs. Constructs 26-29 (composition indicated in Table 1) were expressed in 50 ml culture of wild type Pichia pastoris (i.e. NCYC2543) and purified from the supernatant through gravity flow IMAC and subsequent desalting. 20 μl of the flow through (FT) and wash (W) fractions and 1 μg of the purified protein (P) were analyzed on SDS-PAGE. ‘MM’=molecular weight marker (Precision Plus Protein Standard, Bio-Rad)

FIGS. 5A-5C. In vitro EGFR internalization efficacy of VHH-based nanoLYTAC constructs as determined by flow cytometry. Hela cells were treated with 5 or 50 nM of the nanoLYTAC constructs (26-27) or controls during 24 h. Live cells were stained for cell-surface EGFR (PE-AF647) and measured on the BD LSR II flow cytometer. (FIG. 5A) Representative flow cytometry histograms of cell-surface EGFR levels measured for untreated Hela cells or for Hela cells treated with 5 nM of nanoLYTAC constructs 26 (9G8 S54A-VHH8) or 27 (2x9G8 S54A-VHH8) or with the corresponding control constructs 28 (9G8 S54A-GBP) or 29 (2x9G8 S54A-GBP) respectively. (FIG. 5B) Representative flow cytometry histograms of cell-surface EGFR levels measured for untreated Hela cells or for Hela cells treated with 50 nM of nanoLYTAC constructs 26 (9G8 S54A-VHH8) or 27 (2x9G8 S54A-VHH8) or with the corresponding control constructs 28 (9G8 S54A-GBP) or 29 (2x9G8 S54A-GBP) respectively. (FIG. 5C) Bar plot indicating the median fluorescence intensity values measured for each condition. Data are the mean of two replicates±SEM.

FIG. 6. Western Blot analysis for detection of total EGFR in Hela cell lysates. Hela cells were treated in duplicate with 50 nM of construct 26 (9G8 S54A-VHH8), 27 (2x9G8 S54A-VHH8), 28 (9G8 S54A-GBP) or 29 (2x9G8 S54A-GBP) or left untreated (UT) during 24 hours. As positive control for EGFR degradation, cells were treated with 50 ng/ml of recombinant human EGF. Cell lysates were obtained and immunoblotted for EGFR and beta-actin. ‘kDa’=kilodalton.

FIGS. 7A-7F. Primary images of the live-cell imaging experiments. (FIGS. 7A-7F) Show a particular VHH (i.e. VHH7, −1, −5, −8, negative control (GBP) or recombinant human acid α-glucosidase (rhGAA), used as positive control) that were fluorescently labelled to Alexa Fluor 488. For each image, the most appropriate Z-stack was selected at 120 minutes of incubation and intracellular protein (green) was shown together with the LysoTracker (magenta) and bright-field signal. Imaging was performed on the Zeiss Spinning Disk microscope with the Plan-Apochromat 40× (1.40 oil DIC UV-Vis-IR M27) objective.

FIGS. 8A-8C. Microscopic analysis of internalized and intralysosomal anti-CI-M6PR VHH7 and VHH8. Alexa Fluor 488 (AF488)-labelled VHHs were incubated for four hours on Hela cells (37° C.) and stained with an anti-LAMP1 antibody that was detected using a DyLight594 coupled antibody. (FIG. 8A) Percentage of endocytosed anti-CI-M6PR VHH-AF488, detected in LAMP1-positive lysosomes. (FIG. 8B) Percentage of LAMP1-stained lysosomes, containing VHH7 and VHH8. (FIG. 8C) Images corresponding to AF488-VHH7, AF488-VHH8 treated and untreated (medium) cells (green) colocalising with LAMP1 (magenta). Nuclei were stained with DAPI (cyan). Imaging of three fields of view was performed for every VHH-AF488 on the LSM880 Airyscan confocal microscope (Zeiss) in SR mode using the 63× objective.

FIGS. 9A-9E. Association-dissociation graphs of humanized VHH7 variants analyzed using Biolayer interferometry (BLI). BLI was performed on an Octet Red96 (FortéBio) instrument in kinetics buffer (0.2 M Na₂HPO₄, 0.1 M Na⁺citrate, 0.01% bovine serum albumin, 0.002% Tween-20). Biotinylated human domain₁₋₃His₆ was immobilized on Streptavidin SA biosensors (Sartorius) to a signal of 0.6 nm. A 120 s association phase in VHH7 (FIG. 9A), VHH7h1 (FIG. 9B), VHH7h2 (FIG. 9C), VHH7h3 (FIG. 9D) or VHH7hWN (FIG. 9E) serially diluted (0-200 nM) in pH 7.4 phosphate citrate buffer, was followed by 420 s of dissociation in phosphate buffer at either pH 7.4, 6.5, 6.0, 5.5 or 5.0. Between runs, biosensors were regenerated by three times 10 s exposure to regeneration buffer (10 mM glycine pH 3). The degree of association and dissociation was measured in δ nm over time(s). Black curves represent the double reference-subtracted data that were fitted according to the 1:1 binding model (grey dashed line).

FIGS. 10A-10E. Association-dissociation graphs of humanized VHH8 variants analyzed using Biolayer interferometry (BLI). BLI was performed on an Octet Red96 (FortéBio) instrument in kinetics buffer (0.2 M Na₂HPO₄, 0.1 M Na⁺citrate, 0.01% bovine serum albumin, 0.002% Tween-20). Biotinylated human domain₁₋₃His₆ was immobilized on Streptavidin SA biosensors (Sartorius) to a signal of 0.6 nm. A 120 s association phase in VHH8 (FIG. 10A), VHH8h1 (FIG. 10B), VHH8h2 (FIG. 10C), VHH8h3 (FIG. 10D) or VHH8hWN (FIG. 10E) serially diluted (0-200 nM) in pH 7.4 phosphate citrate buffer, was followed by 420 s of dissociation in phosphate buffer at either pH 7.4, 6.5, 6.0, 5.5 or 5.0. Between assays, biosensors were regenerated by three times 10 s exposure to regeneration buffer (10 mM glycine pH 3). The degree of association and dissociation was measured in δ nm over time(s). Black curves represent the double reference-subtracted data that were fitted according to the 1:1 binding model (grey dashed line).

FIG. 11. Amino acid sequence alignment of CI-M6PR domains 1-3 for human, mouse and bovine proteins and indication of the VHH7/1H11 and VHH8 epitope residues. Bovine (B/, Bos taurus), human (H/, Homo sapiens) and mouse (M/, Mus musculus) CI-M6PR Domain 1-3 sequences multiple alignment, showing the three different domains of the antigen, Domain 1 (D1; bovine residues 49-171), domain 2 (D2; bovine res. 172-325) and domain 3 (D3; bovine res. 326-476). Full circles represent the core epitope residues selected based on integrating the outputs of the 4 Angstrom distance of the VHH, PISA and FastContact analysis. Half circles define further residues within 4 Angstrom distance of the VHH.

FIGS. 12A-12D. Cartoon presentation of the co-crystal structure of VHH7 and domains 1-3 of the hCI-M6PR. (FIG. 12A) VHH7 is coloured in black with its paratope residues (shown as sticks), facing domain 1 (D1) of the CI-M6PR (grey). A detailed figure of the CI-M6PR epitope of VHH7 is shown in FIG. 12B and FIG. 12C. (FIG. 12B) Detailed interface of CI-M6PR D1, displayed as a surfaced cartoon, and sticked paratope residues of CDR1, -2 and -3 of VHH7. (FIG. 12C) Detailed interface of VHH7, displayed as a surfaced cartoon and the epitope residues of CI-M6PR D1 shown as sticks. (FIG. 12D) Shows the paratope residues of VHH7 (black) within less than 4 Å from the epitope region on D1 (grey).

FIGS. 13A-13D. Cartoon presentation of the co-crystal structure of VHH8 and domains 1-3 of the hCI-M6PR. (FIG. 13A) VHH8 is coloured in black with its paratope facing domain 2 (D2) and D3 of the CI-M6PR (grey). A detailed figure of the CI-M6PR epitope of VHH8 is shown in FIG. 13B and FIG. 13C. (FIG. 13B) Detailed interface of CI-M6PR D2 and D3, displayed as a surfaced cartoon (light grey), and sticked paratope residues of CDR1, -2 and -3 of VHH7 (dark grey). (FIG. 13C) Detailed interface of VHH8, displayed as a surfaced cartoon and the epitope residues of CI-M6PR D2 and D3 shown as sticks. (FIG. 13D) Shows the paratope residues of VHH8 (black) within less than 4 Å from the epitope region on D1 (grey).

FIGS. 14A-14D. Cartoon presentation of the co-crystal structure of VHH 1H11 and domains 1-3 of the hCI-M6PR. (FIG. 14A) VHH 1H11 is coloured in black with its paratope residues (shown as sticks), facing domain 1 (D1) of the CI-M6PR (grey). A detailed figure of the CI-M6PR epitope of VHH 1H11 is shown in FIG. 14B and FIG. 14C. (FIG. 14B) Detailed interface of CI-M6PR D1, displayed as a surfaced cartoon, and sticked paratope residues of CDR1, -2 and -3 of VHH 1H11. (FIG. 14C) Detailed interface of VHH 1H11, displayed as a surfaced cartoon and the epitope residues of CI-M6PR D1 shown as sticks. (FIG. 14D) Shows the paratope residues of VHH 1H11 (black) within less than 4 Å from the epitope region on D1 (grey).

FIG. 15. Schematic presentation of the binding of anti-CI-M6PR VHHs to domains 1-3 of the hCI-M6PR. (A) The trefoil-shaped structure of CI-M6PR_{D1-D3} (similar to PDB: 1q25) presented schematically (white) with VHH7 and VHH8 bound to either D1 and D2-D3 respectively (grey). (B) Same as A but with CI-M6PR_{D1-D3} being similar to PDB: 6p8i and binding VHH 1H11 to D1 (grey).

FIG. 16. Crystal structure information of N-terminal three domains of the cation-independent mannose-6-phosphate receptor in complex with anti-CI-M6PR VHH7. Observed crystal contacts in the VHH7: hCI-M6PR_{D1-D3} structure; crystal packing enabled by Asn112-linked glycan of one protein and the M6P-binding pocket in hCI-M6PR_{D3} of another protein. Figures were created in PyMol 2.3.3.

FIG. 17-18. In-tandem competitive BLI of purified anti-CI-M6PR VHHs. In-tandem competitive BLI was performed on an Octet Red96 (FortéBio) instrument in kinetics buffer (1×PBS, 1 mg/ml bovine serum albumin, 0.02% Tween-20 and 0.05% sodium azide). Human CI-M6PR domain₁₋₃His₆ (0.5 mg/ml in 50 mM MES, 150 mM NaCl, pH 6.5) was incubated for 30 minutes at room temperature with EZ-Link™ NHS-PEG4-Biotin (1 mg, Thermo Fischer A39259) and NaHCO₃ (100 mM). Biotinylated human domain₁₋₃His₆ was purified using a Zeba spin desalting column™ (7K MWCO, 2 mL, Thermo Fischer 89890) and immobilized on Streptavidin SA biosensors (Sartorius) to a signal of 0.5 nm In a competitive assay (left), a 60 s association phase in 400 nM purified VHH7 (top) or VHH8 (bottom) was followed by a second association phase in: 400 mM of one of a range of anti-CI-M6PR VHHs recombinantly produced in and purified from E. coli (FIG. 17), or in a periplasmic extract of E. coli expressing one of a range of anti-CI-M6PR VHHs (FIG. 18). In a second reverse assay (right), a 60 s association phase either in 400 nM anti-CI-M6PR-VHH recombinantly produced in and purified from E. coli (FIG. 17), or in a periplasmic extract of E. coli expressing one of a range of anti-CI-M6PR VHHs (FIG. 18) was followed by a second 60 s association phase in 400 nM VHH7 or VHH8. Between assays, biosensors were regenerated by three times 10 s exposure to regeneration buffer (10 mM glycine pH 3). Data were double reference-subtracted and aligned in Octet Data Analysis software v9.0 (FortéBio). Greyscale curves represent double reference-subtracted data. A competition table indicates which combinations of saturating and competing VHHs (all at 400 nM in FIG. 17) resulted in competition or non-blocking interactions.

FIG. 19-20. Association-dissociation graphs of anti-CI-M6PR VHH1H11 and VHH1H52, resp. analyzed using BLI. BLI was performed on an Octet Red96 (FortéBio) instrument in kinetics buffer (0.2 M Na₂HPO₄, 0.1 M Na⁺citrate, 0.01% bovine serum albumin, 0.002% Tween-20). Biotinylated human domain₁₋₃His₆ was immobilized on Streptavidin SA biosensors (Sartorius) to a signal of 0.6 nm. A 120 s association phase in VHH 1H11 (FIG. 19) or VHH 1H52 (FIG. 20) serially diluted (0-200 nM) in pH 7.4 phosphate citrate buffer, was followed by 420 s of dissociation in phosphate buffer of either pH 7.4, 6.5, 6.0, 5.5 or 5.0. Between assays, biosensors were regenerated by three times 10 s exposure to regeneration buffer (10 mM glycine pH 3). The degree of association and dissociation was measured in δ nm over time(s). Black curves represent the double reference-subtracted data that were fitted according to the 1:1 binding model (grey dashed line).

FIG. 21. Amino acid sequences of VHH7 and VHH8 with annotated CDRs. Kabat numbering is used for numbering of the amino acid residues. The Complementary-determining-regions 1, 2 and 3 (CDR1,2, 3) are indicated as grey labelled boxed, according to AbM, MacCallum, Chothia, IMGT or Kabat annotation.

FIGS. 22A and 22B. Coomassie Brilliant Blue-stained SDS-PAGE of VHH-based anti-EGFR nanoLYTAC constructs and controls produced in Pichia pastoris, both with (FIG. 22A) and without (FIG. 22B) dithiothreitol in the Laemmli sample buffer. ‘MM’=molecular weight marker. Construct 30=VHH7-FLAG3His6. Construct 31=VHH8-FLAG3His6. Construct 33=9G8 S54A-FLAG3His6. Construct 34=9G8 S54A-VHH7-FLAG3His6. Construct 35=9G8 S54A-VHH8-FLAG3His6. Construct 36=2x9G8 S54A-VHH7-FLAG3His6. Construct 37=2x9G8 S54A-VHH8-FLAG3His6. Construct 38=9G8 S54A-GBP-FLAG3His6. Construct 39=2x9G8 S54A-GBP-FLAG3His6.

FIGS. 23A-23C. In vitro EGFR internalization efficacy of VHH-based nanoLYTAC constructs as determined by flow cytometry. Hela cells were treated with 50 nM of the nanoLYTAC constructs (34-37) or controls during 24 h. Live cells were stained for cell-surface EGFR (PE-AF647) and measured on the BD LSR II flow cytometer. (FIG. 23A) Representative flow cytometry histogram of cell-surface EGFR levels measured for untreated Hela cells or for Hela cells treated with 50 nM of nanoLYTAC constructs 34 (9G8 S54A-VHH7) or 35 (9G8 S54A-VHH8) or with the corresponding control construct 38 (9G8 S54A-GBP) or 50 ng/ml of recombinant human EGF (rhEGF). (FIG. 23B) Representative flow cytometry histogram of cell-surface EGFR levels measured for untreated Hela cells or for Hela cells treated with 50 nM of nanoLYTAC constructs 36 (2x9G8 S54A-VHH7) or 37 (2x9G8 S54A-VHH8) or with the corresponding control construct 39 (2x9G8 S54A-GBP) or 50 ng/ml rhEGF. (FIG. 23C) Bar plot indicating the median fluorescence intensity values measured for each condition, normalized relative to the median fluorescence intensity of untreated Hela cells and expressed in percentages. Erbitux, FDA/EMA-approved monoclonal anti-EGFR antibody. Data are the mean of two replicates±SEM. The indicated asterisks represent p-values obtained from unpaired t-tests comparing the LYTAC-treated conditions with the untreated (in black) and the control construct (38 or 39)-treated conditions (in grey). * P≤0.05. ** P≤0.01. *** P≤0.001. **** P≤0.0001.

FIGS. 24A-24C. Western Blot-assay to evaluate the in vitro EGFR degradation efficacy of VHH-based nanoLYTAC constructs. Hela cells were treated with 50 nM of the nanoLYTAC constructs (34-37), control constructs (38-39) or with 50 ng/ml of recombinant human EGF (rhEGF) during 24 h. Cell lysates were obtained and immunoblotted for EGFR and beta-tubulin. Intensity values for EGFR were determined through densitometry, normalized to loading control and expressed relative to the untreated or the construct 38-treated condition. (FIG. 24A) Western Blot analysis of 1^{st} biological replicate. (FIG. 24B) Western Blot analysis of 2^{nd} biological replicate. (FIG. 24C) Western Blot analysis of 3^{rd} biological replicate. ‘kDa’=kilodalton. ‘r’=biological replicate

FIG. 25. In vitro inhibition of ligand-induced EGFR activation in response to treatment with VHH-based nanoLYTAC constructs. Hela cells were treated with 50 nM of the nanoLYTAC constructs (34-37), control constructs (38-39) or Erbitux (50 nM or 40 μg/ml) during 24 h, after which cells were stimulated with 50 ng/ml recombinant human EGF (rhEGF) during 5 minutes. Cell lysates were obtained and immunoblotted for phospho-EGFR (Tyr1068). A Ponceau S-stain of the membrane is shown to demonstrate total protein levels. ‘UT’=untreated. ‘Ebx’=Erbitux (FDA/EMA-approved monoclonal anti-EGFR antibody). ‘kDa’=kilodalton.

FIG. 26. Coomassie Brilliant Blue-stained SDS-PAGE of cetuximab-based anti-EGFR nanoLYTAC constructs and cetuximab produced in Chinese hamster ovary (CHO) cells, both with and without dithiothreitol in the Laemmli sample buffer. ‘MM’=molecular weight marker. ‘Ctx-VHH7’=cetuximab-VHH7 fusion construct. ‘Ctx-VHH8’=cetuximab-VHH8 fusion constructs. ‘Ctx’=cetuximab.

FIGS. 27A and 27B. In vitro EGFR internalization efficacy of cetuximab-VHH fusions as LYTAC constructs as determined by flow cytometry. Hela cells were treated with 5 or 50 nM of the cetuximab-based nanoLYTAC constructs (Ctx-VHH7 or Ctx-VHH8) or controls during 24 h. Live cells were stained for cell-surface EGFR (PE-AF647) and measured on the BD LSR II flow cytometer. (FIG. 27A) Representative flow cytometry histograms of cell-surface EGFR levels measured for untreated Hela cells or for Hela cells treated with 5 nM of the cetuximab-based nanoLYTAC constructs or cetuximab or with 50 ng/ml or recombinant human EGF (rhEGF). (FIG. 27B) Bar plot indicating the median fluorescence intensity values measured for each condition, normalized relative to the median fluorescence intensity of untreated HeLa cells and expressed in percentages. Data are the mean of two replicates±SEM. Erbitux=FDA/EMA-approved monoclonal anti-EGFR antibody. The indicated asterisks represent p-values obtained from unpaired t-tests comparing the LYTAC-treated conditions with the untreated (in black) and the cetuximab-treated conditions (in grey). * P≤0.05. * $P≤0.01. *** P≤0.001. **** P≤0.0001.

FIG. 28. Western Blot-assay to evaluate the in vitro EGFR degradation efficacy of cetuximab-VHH fusions as nanoLYTAC constructs. In two independent experiments, Hela cells were treated with 5 nM of the LYTAC constructs (Ctx-VHH7 or Ctx-VHH8), cetuximab or 50 ng/ml of recombinant human EGF (rhEGF) during 24 h. Cell lysates were obtained and immunoblotted for EGFR and beta-tubulin. Intensity values for EGFR were determined through densitometry, normalized to loading control and expressed relative to the untreated or cetuximab-treated condition. ‘kDa’=kilodalton. ‘Ctx-VHH7’=cetuximab-VHH7 fusion constructs. ‘Ctx-VHH8’=cetuximab-VHH8 fusion construct. ‘Ctx’=cetuximab. ‘r’=biological replicate.

FIG. 29. Coomassie Brilliant Blue-stained SDS-PAGE of VHH-based anti-GFP nanoLYTAC constructs and controls produced in Pichia pastoris. ‘MM’=molecular weight marker. Construct 42=GBP-FLAG3His6. Construct 43=GBP-VHH7-FLAG3His6. Construct 44=GBP-VHH8-FLAG3His6. Construct 45=GBP-VHH1-FLAG3His6. Construct 46=GBP-VHH5-FLAG3His6. Construct 47=GBP-VHH 1H11-FLAG3His6. Construct 48=GBP-VHH 1H52-FLAG3His6.

FIG. 30. Western Blot assay to evaluate in vitro GFP internalization and degradation in Hela cells treated with anti-GFP nanoLYTAC constructs. Hela cells were treated during 24 h with 50 nM of recombinant GFP (rGFP) and 50 nM of the nanoLYTAC constructs (43=GBP-VHH7 and 44=GBP-VHH8) or of the control construct (42=GBP) with or without chloroquine. Cell lysates were obtained and immunoblotted for GFP and beta-tubulin. As a positive control, 2.5 ng of rGFP was analyzed. ‘UT’=untreated. ‘kDa’=kilodalton. ‘CQ’=chloroquine’.

FIG. 31. Western Blot assay to evaluate in vitro GFP internalization and degradation in MCF7 cells treated with anti-GFP nanoLYTAC constructs. MCF7 cells were treated during 24 h with 200 nM of recombinant GFP and 200 nM of the nanoLYTAC constructs (43=GBP-VHH7 and 44=GBP-VHH8) or of the control construct (42=GBP) with or without chloroquine. Cell lysates were obtained and immunoblotted for GFP and beta-tubulin. ‘UT’=untreated. ‘kDa’=kilodalton. ‘CQ’=chloroquine’.

FIG. 32. Western Blot assay to evaluate in vitro GFP internalization and degradation in Hela cells treated with anti-GFP nanoLYTAC constructs. Hela cells were treated during 24 h with 200 nM of recombinant GFP (rGFP) and 200 nM of the nanoLYTAC constructs (43=GBP-VHH7, 44=GBP-VHH8, 45=GBP-VHH1, 46=GBP-VHH5, 47=GBP-VHH 1H11, 48=GBP-VHH 1H52) or of the control construct (42=GBP). Cell lysates were obtained and immunoblotted for GFP and beta-tubulin. ‘UT’=untreated. ‘kDa’=kilodalton. ‘CQ’=chloroquine’.

FIG. 33. Western Blot assay to evaluate in vitro GFP internalization and degradation in Hela cells after washout of anti-GFP nanoLYTAC treatment. Hela cells were treated during 24 h with 50 nM of recombinant GFP (rGFP) and 50 nM of the nanoLYTAC constructs (43=GBP-VHH7 and 44=GBP-VHH8) or of the control construct (42=GBP) with or without chloroquine. Cell lysates were obtained after treatment (+0h) and after an additional 3 (+3h) and 7 (+7h) hours of incubation in fresh growth medium. The lysates were immunoblotted for GFP and beta-tubulin. As a positive control, 2.5 ng of rGFP was analyzed. ‘UT’=untreated. ‘kDa’=kilodalton. ‘CQ’=chloroquine’.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein. The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases ‘in one embodiment’ or ‘in an embodiment’ in various places throughout this specification are not necessarily all referring to the same embodiment but may.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. Unless defined otherwise, all technical and scientific terms used herein have the same meaning as commonly understood by one of ordinary skill in the art (e.g. in molecular biology, biochemistry, structural biology, and/or computational biology).

“Nucleotide sequence”, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, the (reverse) complement DNA, and RNA. It also includes known types of modifications, for example, methylation, “caps” substitution of one or more of the naturally occurring nucleotides with an analog. By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like. “Coding sequence” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances. The term “vector”, “vector construct,” “expression vector”, or “recombinant vector” as used herein, is intended to refer to a nucleic acid molecule capable of transporting another nucleic acid molecule to which it has been linked. More particular, said vector may include any vector known to the skilled person, including any suitable type, but not limited to, for instance, plasmid vectors, cosmid vectors, phage vectors, such as lambda phage, viral vectors, even more particular a lentiviral, adenoviral, AAV or baculoviral vectors, or artificial chromosome vectors such as bacterial artificial chromosomes (BAC), yeast artificial chromosomes (YAC), or P1 artificial chromosomes (PAC). Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments. The construction of expression vectors for use in transfecting cells is also well known in the art, and thus can be accomplished via standard techniques (see, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, Tex.).

The terms “protein”, “polypeptide”, and “peptide” are interchangeably used further herein to refer to a polymer of amino acid residues and to variants and synthetic analogues of the same. A “peptide” may also be referred to as a partial amino acid sequence derived from its original protein, for instance after tryptic digestion. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. This term also includes posttranslational modifications of the polypeptide, such as glycosylation, phosphorylation and acetylation. Based on the amino acid sequence and the modifications, the atomic or molecular mass or weight of a polypeptide is expressed in (kilo) dalton (kDa). By “isolated” or “purified” is meant material that is substantially or essentially free from components that normally accompany it in its native state. For example, an “isolated polypeptide” or “purified polypeptide” refers to a polypeptide which has been purified from the molecules which flank it in a naturally-occurring state, e.g., a protein binding agent such as a fusion protein or antibody or nanobody as identified and disclosed herein which has been removed from the molecules present in the sample or mixture, such as a production host, that are adjacent to said polypeptide. An isolated protein or peptide can be generated by amino acid chemical synthesis or can be generated by recombinant production or by purification from a complex sample.

“Homologue”, “Homologues”, or “functional homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue occurs in both sequences to yield the number of matched positions, dividing the number of matched positions by the total number of positions in the window of comparison (i.e., the window size), and multiplying the result by 100 to yield the percentage of sequence identity. A “substitution”, or “mutation”, or “variant” as used herein, results from the replacement of one or more amino acids or nucleotides by different amino acids or nucleotides, respectively as compared to an amino acid sequence or nucleotide sequence of a parental protein or a fragment thereof.

It is understood that a protein or a fragment thereof may have conservative amino acid substitutions which have substantially no effect on the protein's activity or functionality.

Amino acids are presented herein by their 3- or 1-lettercode nomenclature as defined and provided also in the IUPAC-IUB Joint Commission on Biochemical Nomenclature (Nomenclature and Symbolism for Amino Acids and Peptides. Eur. J. Biochem. 138:9-37 (1984)); as follows: Alanine (A or Ala), Cysteine (C or Cys), Aspartic acid (D or Asp), Glutamic acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr).

“Binding” means any interaction, be it direct or indirect. A direct interaction implies a contact between the binding partners. An indirect interaction means any interaction whereby the interaction partners interact in a complex of more than two molecules. The interaction can be completely indirect, with the help of one or more bridging molecules, or partly indirect, where there is still a direct contact between the partners, which is stabilized by the additional interaction of one or more molecules. By the term “specifically binds,” as used herein is meant a binding domain which recognizes a specific target, but does not substantially recognize or bind other molecules in a sample. Specific binding does not mean exclusive binding. However, specific binding does mean that proteins have a certain increased affinity or preference for one or a few of their binders. The term “affinity”, as used herein, generally refers to the degree to which a ligand, chemical, protein or peptide binds to another (target) protein or peptide so as to shift the equilibrium of single protein monomers toward the presence of a complex formed by their binding. Affinity is the strength of binding of a single molecule to its ligand. It is typically measured and reported by the equilibrium dissociation constant (K_{D}), which is used to evaluate and rank order strengths of bimolecular interactions. The binding of an antibody to its antigen is a reversible process, and the rate of the binding reaction is proportional to the concentrations of the reactants. At equilibrium, the rate of [antibody] [antigen] complex formation is equal to the rate of dissociation into its components [antibody]+[antigen]. The measurement of the reaction rate constants can be used to define an equilibrium or affinity constant (1/K_{D}). In short, the smaller the K_{D} value the greater the affinity of the antibody for its target. The rate constants of both directions of the reaction are termed: the association reaction rate constant (Kₒₙ), which is the part of the reaction used to calculate the “on-rate” (Kₒₙ), a constant used to characterize how quickly the antibody binds to its target. Vice versa, the dissociation reaction rate constant (K_{off}), is the part of the reaction used to calculate the “off-rate” (K_{off}), a constant used to characterize how quickly an antibody dissociates from its target. In measurements as shown herein, the flatter the slope, the slower off-rate, or the stronger antibody binding. Vice versa, the steeper downside indicates a faster off-rate and weaker antibody binding. The ratio of the experimentally measured off- and on-rates (K_{off}/Kₒₙ) is used to calculate the K_{D} value. Several determination methods are known to the skilled person to measure on and off rates and to thereof calculate the K_{D}, which is therefore, taking into account standard errors, considered as a value that is independent of the assay used. As used herein, the term “protein complex” or “complex” or “assembled protein(s)” refers to a group of two or more associated macromolecules, whereby at least one of the macromolecules is a protein. A protein complex, as used herein, typically refers to associations of macromolecules that can be formed under physiological conditions. Individual members of a protein complex are linked by non-covalent interactions.

A “binding agent” relates to a molecule that is capable of binding to another molecule, wherein said binding is preferably a specific binding, recognizing a defined binding site, pocket or epitope. A binding agent may also be provided as a (covalent) complex of several molecules, such as an antibody or alike. The binding agent may be of any nature or type and is not dependent on its origin. The binding agent may be chemically synthesized, naturally occurring, recombinantly produced (and purified), as well as designed and synthetically produced. Said binding agent may hence be a small molecule, a chemical, a peptide, a polypeptide, an antibody, or any derivatives thereof, such as a peptidomimetic, an antibody mimetic, an active fragment, a chemical derivative, among others. The protein binding agent as disclosed herein is a polypeptide, which is in itself also composed of fusion protein comprising a first binding agent, specifically a CI-M6PR-specific ISVD as described herein, and a second binding agent, specifically binding an extracellularly-accessible target protein. In specific embodiments, said second binding agent of the fusion protein may require further components, such as an antibody light chain, as to form the binding site for the extracellularly-accessible target protein, as a whole, together with the fusion protein forming the protein binding agent of the invention. The term “binding pocket” or “binding site” refers to a region of a molecule or molecular complex, that, as a result of its shape and charge, favourably associates with another chemical entity, compound, proteins, peptide, antibody or Nb. The term “pocket” includes, but is not limited to cleft, channel or site. The term “part of a binding pocket/site” refers to less than all of the amino acid residues that define the binding pocket, or binding site. For example, the portion of residues may be key residues that play a role in ligand binding, or may be residues that are spatially related and define a three-dimensional compartment of the binding pocket. The residues may be contiguous or non-contiguous in primary sequence. For antibody-related molecules, the term “epitope” is also used to describe the binding site, as used interchangeably herein. Methods of determining the spatial conformation of amino acids are known in the art, and include, for example, X-ray crystallography, Cryo-EM, and multi-dimensional nuclear magnetic resonance.

The term “antibody”, “antibody fragment” and “active antibody fragment” as used herein refer to a protein comprising an immunoglobulin (Ig) domain or an antigen binding domain capable of specifically binding the antigen, in this case the N-terminal domains 1-3 of the (human) CI-M6PR protein. ‘Antibodies’ can further be intact immunoglobulins derived from natural sources or from recombinant sources and can be immunoreactive portions of intact immunoglobulins. Antibodies are typically tetramers of immunoglobulin molecules. The term “active antibody fragment” refers to a portion of any antibody or antibody-like structure that by itself has high affinity for an antigenic determinant, or epitope, and contains one or more complementarity-determining-regions (CDRs) accounting for such specificity. Non-limiting examples include immunoglobulin domains, Fab, F (ab)′2, scFv, heavy-light chain dimers, immunoglobulin single variable domains, Nanobodies, domain antibodies, and single chain structures, such as a complete light chain or complete heavy chain. An additional requirement for “activity” of said fragments in the light of the present invention is that said fragments are capable of binding CI-M6PR, or, in view of the binding agent specifically recognizing the extracellularly-accessible target, being an antibody fragment, the activity includes the capability to specifically bind the extracellularly-accessible target, as such, or after co-expression/in the presence of a further protein domain such as a light chain or light chain variable domain. Preferably said CI-M6PR binding activity includes specifically binding and having favorable dissociation profiles at lower pH (i.e. acidic conditions as in endosomes and lysosomes below pH 7), more preferably are capable to dissociate at a pH around 5.8, and/or retain binding at said pH (depending on the application/treatment) in a subject. The term “immunoglobulin (Ig) domain”, or more specifically “immunoglobulin variable domain” (abbreviated as “IVD”) means an immunoglobulin domain essentially consisting of four “framework regions” which are referred to in the art and herein below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and herein below as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain(s) (IVDs) that confer specificity to an antibody for the antigen by carrying the antigen-binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab fragment, a F (ab′) 2 fragment, an Fv fragment such as a disulphide linked Fv or a scFv fragment, or a diabody (all known in the art) derived from such conventional 4-chain antibody, with binding to the respective epitope of an antigen by a pair of (associated) immunoglobulin domains such as light and heavy chain variable domains, i.e., by a VH-VL pair of immunoglobulin domains, which jointly bind to an epitope of the respective antigen. An immunoglobulin single variable domain (ISVD) as used herein, refers to a protein with an amino acid sequence comprising 4 Framework regions (FR) and 3 complementary determining regions (CDR) according to the format of FR1-CDR1-FR2-CDR2-FR3-CDR3-FR4. An “immunoglobulin domain” of this invention also refers to “immunoglobulin single variable domains” (abbreviated as “ISVD”), equivalent to the term “single variable domains”, and defines molecules wherein the antigen binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. The binding site of an immunoglobulin single variable domain is formed by a single VH/VHH or VL domain. Hence, the antigen binding site of an immunoglobulin single variable domain is formed by no more than three CDR's. As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

In particular, the immunoglobulin single variable domain may be a Nanobody® (as defined herein) or a suitable fragment thereof. Note: Nanobody®, Nanobodies® and Nanoclone® are registered trademarks of Ablynx N.V. (a Sanofi Company). For a general description of Nanobodies, reference is made to the further description below, as well as to the prior art cited herein, such as e.g. described in WO2008/020079. “VHH domains”, also known as VHHs, VHH domains, VHH antibody fragments, and VHH antibodies, have originally been described as the antigen binding immunoglobulin (Ig) (variable) domain of “heavy chain antibodies” (i.e., of “antibodies devoid of light chains”; Hamers-Casterman et al (1993) Nature 363:446-448). The term “VHH domain” has been chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHHs and Nanobody, reference is made to the review article by Muyldermans (Reviews in Molecular Biotechnology 74:277-302, 2001), as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1433793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. As described in these references, Nanobody (in particular VHH sequences and partially humanized Nanobody) can in particular be characterized by the presence of one or more “Hallmark residues” in one or more of the framework sequences. A further description of the Nanobody, including humanization and/or camelization of Nanobody, as well as other modifications, parts or fragments, derivatives or “Nanobody fusions”, multivalent or multispecific constructs (including some non-limiting examples of linker sequences) and different modifications to increase the half-life of the Nanobody and their preparations can be found e.g. in WO 08/101985 and WO 08/142164. Nanobodies form the smallest antigen binding fragment that completely retains the binding affinity and specificity of a full-length antibody. Nbs possess exceptionally long complementarity-determining region 3 (CDR3) loops and a convex paratope, which allow them to penetrate into hidden cavities of target antigens.

As used herein, the terms “determining,” “measuring,” “assessing,”, “identifying”, “screening”, and “assaying” are used interchangeably and include both quantitative and qualitative determinations.

A “pharmaceutically or therapeutically effective amount” of protein binding agent or binding agent composition is preferably that amount which produces a result or exerts an influence on the particular condition being treated. A “therapeutically active agent” is used to refer to any molecule that has or may have a therapeutic effect (i.e. curative or stabilizing effect) in the context of treatment of a disease (as described further herein). Preferably, a therapeutically active agent is a disease-modifying agent, and/or an agent with a curative effect on the disease. By “pharmaceutically acceptable” is meant a material that is not biologically or otherwise undesirable, i.e., the material may be administered to an individual along with the compound without causing any undesirable biological effects or interacting in a deleterious manner with any of the other components of the pharmaceutical composition in which it is contained. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. Suitable carriers or adjuvantia typically comprise one or more of the compounds included in the following non-exhaustive list: large slowly metabolized macromolecules such as proteins, polysaccharides, polylactic acids, polyglycolic acids, polymeric amino acids, amino acid copolymers and inactive virus particles. Such ingredients and procedures include those described in the following references, each of which is incorporated herein by reference: Powell, M. F. et al. (“Compendium of Excipients for Parenteral Formulations” PDA Journal of Pharmaceutical Science & Technology 1998, 52 (5), 238-311), Strickley, R. G (“Parenteral Formulations of Small Molecule Therapeutics Marketed in the United States (1999)—Part-1” PDA Journal of Pharmaceutical Science & Technology 1999, 53 (6), 324-349), and Nema, S. et al. (“Excipients and Their Use in Injectable Products” PDA Journal of Pharmaceutical Science & Technology 1997, 51 (4), 166-171). The term “excipient”, as used herein, is intended to include all substances which may be present in a pharmaceutical composition and which are not active ingredients, such as salts, binders (e.g., lactose, dextrose, sucrose, trehalose, sorbitol, mannitol), lubricants, thickeners, surface active agents, preservatives, emulsifiers, buffer substances, stabilizing agents, flavouring agents or colorants. A “diluent”, in particular a “pharmaceutically acceptable vehicle”, includes vehicles such as water, saline, physiological salt solutions, glycerol, ethanol, etc. Auxiliary substances such as wetting or emulsifying agents, pH buffering substances, preservatives may be included in such vehicles.

The term “subject”, “individual” or “patient”, used interchangeably herein, relates to any organism such as a vertebrate, particularly any mammal, including both a human and another mammal, for whom diagnosis, therapy or prophylaxis is desired, e.g., an animal such as a rodent, a rabbit, a cow, a sheep, a horse, a dog, a cat, a lama, a pig, or a non-human primate (e.g., a monkey). The rodent may be a mouse, rat, hamster, guinea pig, or chinchilla. In one embodiment, the subject is a human, a rat or a non-human primate. Preferably, the subject is a human. In one embodiment, a subject is a subject with or suspected of having a disease or disorder, in particular a disease or disorder as disclosed herein, also designated “patient” herein. However, it will be understood that the aforementioned terms do not imply that symptoms are present. The term “treatment” or “treating” or “treat” can be used interchangeably and are defined by a therapeutic intervention that slows, interrupts, arrests, controls, stops, reduces, or reverts the progression or severity of a sign, symptom, disorder, condition, or disease, but does not necessarily involve a total elimination of all disease-related signs, symptoms, conditions, or disorders.

The term “medicament”, as used herein, refers to a substance/composition used in therapy, i.e., in the prevention or treatment of a disease or disorder. According to the invention, the terms “disease” or “disorder” refer to any pathological state, in particular to the diseases or disorders as defined herein.

### DETAILED DESCRIPTION

The present invention is based on the identification of CI-M6PR-specific VHHs fused to a further antigen-binding protein, to enable target binding at the cell surface or extracellular space, and trigger internalisation of the complex of said protein binding agent and said target via the CI-M6P-receptor endocytotic/lysosomal pathway. VHHs were chosen as binding agents to specifically engage with CI-M6PR since they are known as highly stable and soluble, and can easily and cost-effectively be manufactured in lower organisms such as bacteria and yeast. Moreover, VHHs are unique in their great conformational stability, and high intrinsic pH and protease resistance, which all form attractive properties for cycling through the endosomal-lysosomal system. Furthermore, VHH-based formats are suitable for various routes of administration, including via intravenous injection and inhalation, thus providing for a novel approach to apply lysosomal targeting of drug products, optionally in complex with their targets. More specifically, the target binders fused to said CI-M6PR-specific ISVDs or VHHs as described herein may be antigen-binding domains specific for a target protein, preferably a target present on the cell surface or extracellularly, which in itself also provide for antibody-based, preferably, ISVD-based target binding. Such bispecific binders or ISVD-fusion polypeptides also named herein as nanoLYTACs result in CI-M6PR-mediated lysosomal uptake, as cargo for delivery of specific extracellular or cell surface target(s), which will finally be degraded in the lysosomes.

Because the CI-M6PR constantly traffics between the late endosome and the cell membrane, the protein binding agents disclosed herein may dissociate at the lower pH in these subcellular organelles, or may retain binding to CI-M6PR and recycle with it. The latter may contribute to an increased half-life of such binding agents in a subject. Moreover, tunability of pH dissociation of antigen-binding domains is known in the art, and may allow to generate multispecific binders wherein for instance the CI-M6PR-specific ISVD is capable of maintaining its binding throughout the recycling process, while further antigen-domain binders may dissociate from their target at pH values corresponding to pH in the endosome and lysosome, as to release its target for degradation. This would increase their target degradation efficacy and hence potency. Though also a high protease-resistance is required for recycling of such an ISVD-based anti-CI-M6PR binders.

The present invention discloses at least two types of CI-M6PR-specific ISVDs, based on their binding to a specific epitope on the N-terminal domains of CI-M6PR. As exemplified herein, the selection of which of those ISVDs as part of the protein binding agent as described herein is dependent on the combination and choice of extracellularly-accessible target and its binder, since epitope-positions may be relevant for potency, as well as pH-dependency profiles of both, the CI-M6PR binding and the extracellularly-accessible target binding. By providing two types of CI-M6PR ISVDs, each covered by several VHH examples, a toolbox is provided to select from for the skilled person aiming to obtain targeted protein degradation via the CI-M6PR mechanism.

A first aspect of the invention thus provides for a protein binding agent, preferably comprising a fusion protein, comprising an ISVD-based binding agent specifically binding the N-terminal extracellular portion of the CI-M6PR protein, more specifically binding to a conformational epitope present on domains 1, 2 and/or 3 as defined herein, linked to a binding agent specifically binding a target protein which is accessible extracellularly, more specifically a protein that is secreted by the cell or that is a membrane protein, or present on the cell exterior, wherein said binding agents are directly linked, or connected via a spacer or a linker.

The binding agents or fusion proteins of the present inventions are termed ‘fusions’ as the different binding agents are connected by direct fusions, made via peptide bonds between amino acid residues of the chain and ISVD itself, or indirect fusions made by a linker. Said fusion sites preferably being designed to result in flexible fusion protein, wherein the different paratopes do not interfere with each other for binding to their respective target or antigen. Preferred “linker molecules”, “linkers”, or “short polypeptide linkers” are peptides with a length of about ten amino acids. Non-limiting examples of suitable linker sequences are known by the skilled person. Linkers may be selected to keep a fixed distance between the structural domains, as well as to maintain the fusion partners their independent functions (e.g. antigen-binding).

In a specific embodiment, the ‘linker’ between said CI-M6PR-specific ISVD and target-specific binding agent (wherein ‘target’ is used herein a ‘extracellularly-accessible target protein’ as used herein) of the protein binding agent of the invention may be a longer polypeptide linker, as to allow that the at least two different binding sites can be reached or bound simultaneously by the protein binding agent. For instance the CI-M6PR-specific ISVD as described herein may be fused at its N- or C-terminus to an Fc domain, for instance an Fc-tail of an Ig, and the target-specific binding agent may be fused to an identical or compatible Fc-tail via its N- or C-terminus, resulting in a protein binding agent of bispecific format wherein two of said Fc-fusions, form a dimer, as for antibody-type molecules through disulfide bridges in the hinge region of the Fc part. Alternatively, the Fc-tail may be fused on its N- or C-terminus to the CI-M6PR-specific ISVD and the other terminus to the target-specific binder, resulting in a CI-M6PR-ISVD-Fc-target-binder protein binding agent, which may also be formed as dimeric molecules to provide bivalent bispecific agents. Further linker formats include also Fcs with a knob into hole-linkage possibility, wherein again the CI-M6PR-ISVD and target-specific binder or N- or C-terminally fused to said Fcs, to obtain dimeric bispecific binding agents.

In a further embodiment, said linker between said CI-M6PR-specific ISVD and target-specific binding agent of the protein binding agent of the invention may be provided by a further functional group or moiety, advantageous when administrated to a subject. Examples of such functional groups and of techniques for introducing them will be clear to the skilled person, and can generally comprise all functional groups and techniques mentioned in the art as well as the functional groups and techniques known per se for the modification of pharmaceutical proteins, and in particular for the modification of antibodies or antibody fragments, for which reference is for example made to Remington's Pharmaceutical Sciences, 16th ed., Mack Publishing Co., Easton, PA (1980). Such functional groups may for example be linked directly (for example covalently) to the ISVD, and/or target-specific binding agent, or optionally via a further suitable linker or spacer, as will again be clear to the skilled person. Said functional groups may also be applied as a further moiety linked to the CI-M6PR-specific ISVD or to the target-specific binder. One of the most widely used techniques for increasing the half-life and/or reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). For example, for this purpose, PEG may be attached to a cysteine residue that naturally occurs in a immunoglobulin single variable domain of the invention, a immunoglobulin single variable domain of the invention may be modified so as to suitably introduce one or more cysteine residues for attachment of PEG, or an amino acid sequence comprising one or more cysteine residues for attachment of PEG may be fused to the N- and/or C-terminus of an ISVD or active antibody fragment of the invention, all using techniques of protein engineering known per se to the skilled person. Another, usually less preferred modification comprises N-linked or O-linked glycosylation, usually as part of co-translational and/or post-translational modification, depending on the host cell used for expressing the protein binding agent. Another technique for increasing the half-life of a binding domain may comprise the engineering into bifunctional or bispecific domains (for example, at least one target-specific binder, one ISVD or active antibody fragment against the CI-M6PR and one against a serum protein such as albumin aiding in prolonging half-life) or into fusions of antibody fragments, in particular immunoglobulin single variable domains, with peptides (for example, a peptide against a serum protein such as albumin). The half-life extension can thus be applied as a linker between the CI-M6PR-specific ISVD and the target-specific binder, or can be coupled to either one of them.

The binding to the CI-M6PR protein at the extracellular surface of a cell requires a certain affinity, as to maintain its binding upon internalisation of the receptor in the endosomes. Once a threshold binding affinity is reached, which may be in the micromolar, nanomolar, or picomolar range, and the target-specific binder has bound its target, internalisation and uptake of said bispecific agent, in complex with the target in the cell leads to the protein binding agent/target complex being present within the cellular compartments, from early endosomes, to later endosome, to finally go to the lysosomes of the cell. For the CI-M6PR binders of the present invention, a binding affinity in the nanomolar to picomolar range is envisaged, as determined at neutral pH, more specifically at pH 7.4, as to allow efficient uptake and or recycling with the CI-M6PR protein in the cell.

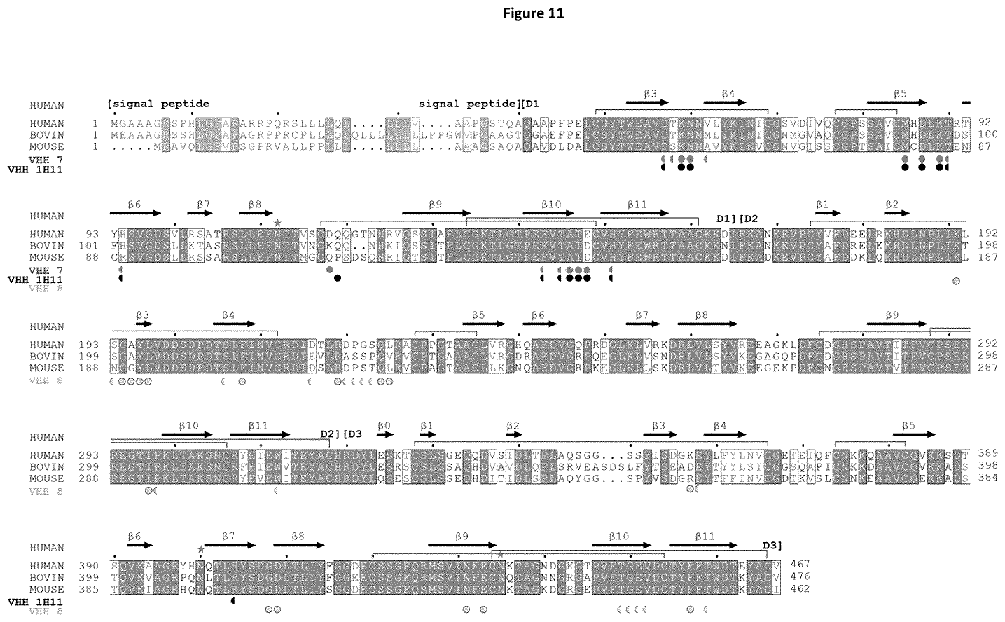

The CI-M6PR-specific ISVD of the protein binding agent of the present invention, specifically binding CI-M6PR at the N-terminal domains 1-3 is defined herein as binding to an epitope that tis present on at least one or more of said 3 N-terminal domains, which are constituting the amino acid residues 1-161 as present in SEQ ID NO:23 for N-terminal domain 1, amino acid residues 162-313 as present in SEQ ID NO: 23 for N-terminal domain 2, and 314-467 as present in SEQ ID NO:23 for N-terminal domain 3 (see for instance FIG. 11). In one embodiment, said CI-M6PR-specific ISVD provides for the necessary biophysical and binding characteristics at different pH values as to retain binding to the CI-M6P receptor N-terminal portion upon internalisation into endosomes and/or lysosome trafficking on or in a cell. In a further specific embodiment, the efficiency of its internalisation is defined as the minimal internalisation rate of said CI-M6PR-specific binding agent by the voxel counts/minute in a life cell imaging experimental method (see Examples), and is herein considered as ‘internalised’ with an internalisation rate of at least 15 voxel counts/min, or at least 35, or at least 50, or at least 65, or at least 80, or at least 100, or at least 120 voxel counts/minute.

In a specific embodiment, said binding agent provides for a retained binding to said CI-M6P receptor upon internalisation, and as shown by its pH dependent binding profile (demonstrated for the ISVDs by BLI), only dissociates from the receptor at a pH below the pH of the endosomal compartment, so below pH 6. Hence said ISVD-based binding agents provide for strong binders at neutral pH and in the endosomes (pH 6-5.5), but allow clear dissociation from the receptor at lower pH, which likely leads to said ISVD-binding agent to at least partially be recycled back to the outer membrane. This may lead to functional ISVD-based removal of surface- or extracellular molecules from the outside of the cell to the endosomal compartments. Such a pH-dependent dissociation profile has for instance been observed for the VHH8 (SEQ ID NO:8), VHH5 (SEQ ID NO:5), and VHH1H52 (SEQ ID NO:25) ISVDs previously disclosed (Callewaert et al., PCT/EP2022/054278). Those VHHs belong to a different VHH family, though, they compete for the same binding site on the CI-M6PR, and based on co-crystal analysis of VHH8 with the CI-M6PR dom1-3, the epitope was determined to be located on N-terminal domains 2 and 3.

Thus in a specific embodiment, said ISVD specifically binding CI-M6PR, specifically recognizes a binding site located on N-terminal domains 2 and 3, wherein said binding site may be more specifically delineated as the ISVD being in contact with the epitope (also called VHH8-petiope) or amino acid residues of CI-M6PR Lys191, Gly194, Ala195, Tyr196, Leu197, Phe208, Arg219, Gln224, Leu225, Ile297, Lys357, Gly408, Asp409, Asn431, Glu433, and Phe457 as depicted in SEQ ID NO:23.

An “epitope”, or “binding site” as used herein, refers to an antigenic determinant of a polypeptide, constituting a binding site or binding pocket on a target molecule, such as the extracellular part of the CI-M6P receptor protein, more specifically a binding pocket on the N-terminal domains (1-3) accessible for the ISVDs or VHHs. An epitope could comprise 3 amino acids in a spatial conformation, which is unique to the epitope. Generally, an epitope consists of at least 4, 5, 6, 7 such amino acids, and more usually, consists of at least 8, 9, 10, or more such amino acids. These residues are in ‘in contact’ with the binding agent. The epitope is defined herein as the amino acids being in contact with each other based on an integrated analysis of a distance of 4 Angstrom or less from the VHH residues, a PISA and a FastContact analysis, as described in Callewaert et al. (PCT/EP2022/054278).

In a further embodiment, said CI-M6PR-specific binding agent may be defined as an agent competing for binding to said VHH8-epitope as described herein.

The binding agent residue specifically binding to the target, or making up the essential residues to bind the epitope of the target are defined herein as the paratope, as known in the art. Such a paratope of a binding agent for CI-M6PR may thus be described as the residues of said ISVD as disclosed herein in contact with the epitope residues on the CI-M6PR N-terminal domains 1-3.

In a further specific embodiment said CI-M6PR-specific ISVD specifically binds by having in contact a specific paratope of said ISVD, which is for instance composed of residues Tyr32, Arg52, Trp53, Ser54, Ser56, Lys57, Ile100, Phe103 and Ser108, as set forth in SEQ ID NO:8 (numerical order, no Kabat numbering is used here) providing for the paratope of said ISVD for binding to said epitope described above. Alternatively, said CI-M6PR-specific ISVD specifically binds by having in contact a specific paratope of VHH5 or VHH1H52 corresponding to said residues 32, 52-57, 100-103, 108 of VHH8, upon sequence alignment.

In a further alternative embodiment, said protein binding agent provides for a CI-M6PR-specific ISVD for internalisation, which, as shown by its pH dependent binding profile (Callewaert et al., PCT/EP2022/054278), gradually dissociates from the receptor at a pH as present in the endosomal compartment, so dissociation occurs similar to the receptor's natural ligands, at a pH around 6 down to 5.5. Hence said ISVD-based binding agents provide for binders at neutral pH but with dissociation in the endosomes (pH 6-5.5), allowing the receptor to cycle back, and the ISVD-binding agent to proceed to the lysosome (and not be recycled to the outer membrane). Such a pH-dependent dissociation profile has for instance been observed for the VHH7 (SEQ ID NO:7), VHH1 (SEQ ID NO:1), and VHH1H11 (SEQ ID NO: 24) ISVDs. Each of those VHHs belong to a different VHH family, though, they compete for the same binding site on the M6PR dom1-3, and based on co-crystal analysis of VHH7 and VHH1H11 with the CI-M6PR dom1-3, the epitope was determined to be located on N-terminal domain 1. More specifically, said CI-M6PR-specific ISVD binding site (herein also referred to as VHH7-epitope or VHH7/VHH1H11 epitope or VHH1H11 epitope) may be more specifically delineated as the ISVD being in contact with the amino acid residues of CI-M6PR at position Lys59, Asn60, Met85, Asp87, Lys89, Ala146, Thr147, and Glu148, and Asp118 or Gln119, as set forth in SEQ ID NO:23. The epitope is defined herein as the amino acids being in contact with each other based on an integrated analysis of a distance of 4 Angstrom or less from the VHH residues, a PISA and a FastContact analysis, as described in Callewaert et al. (PCT/EP2022/054278).

In a further specific embodiment said binding agent comprising an ISVD specifically binding CI-M6PR predominantly domain 1 by having in contact its residues Asp31, Arg33, Asp35, Trp53, Ser54, Ser56, Lys57, Lys96, Asp104, as set forth in SEQ ID NO:7 (numerical order, no Kabat numbering is used here) providing for the paratope of said ISVD for binding to said epitope described above. Alternatively, said CI-M6PR-specific ISVD specifically binds by having in contact a specific paratope of VHH1 or VHH1H11 corresponding to said residues 31, 33, 35, 53, 54, 56, 57, 96, 104 of VHH7, upon sequence alignment, such as for instance the paratope comprising residues 31-35, 50, 52-57, 96-98 as set forth in SEQ ID NO: 24.

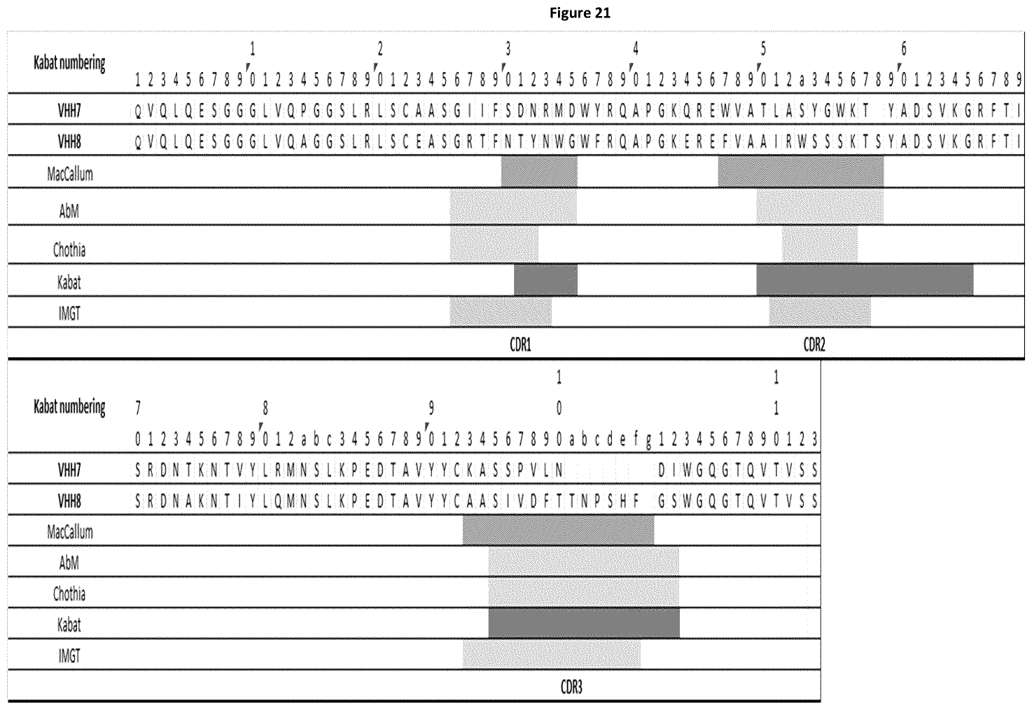

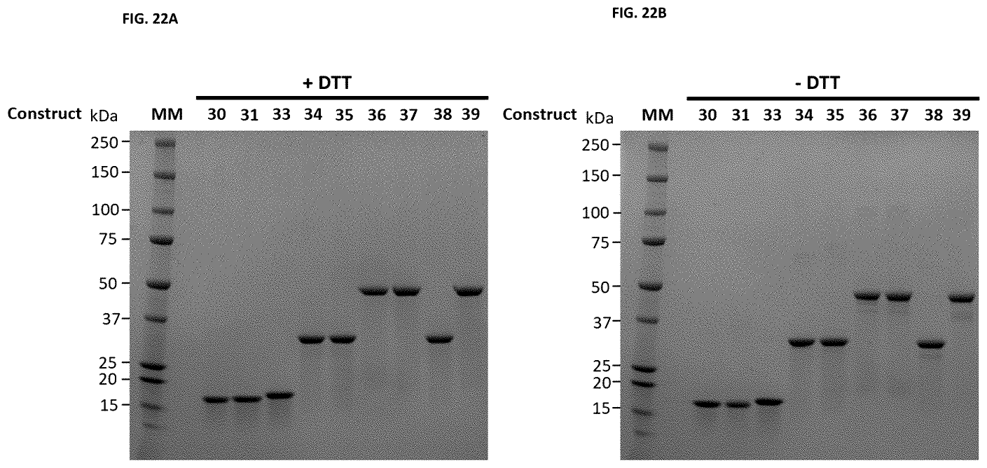

In further embodiments the protein binding agent as described herein comprises the CI-M6PR-specific ISVD comprising a CDR1, CDR2 and CDR3 region, which concern the binding residues of ISVDs, selected from the CDR1, CDR2, and CDR3, respectively of any of the sequences selected from the VHH1, VHH5, VHH7, VHH8, VHH1H11, or VHH1H52 ISVDs wherein said CDR regions are defined according to any one of the annotations known in the art, specifically, according to the annotation of Kabat, MacCallum, IMGT, AbM or Chothia. Determination of CDR regions may be done according to different methods, such as the designation based on contact analysis and binding site topography as described in MacCallum et al. (J. Mol. Biol. (1996) 262, 732-745), or according to any of the annotations known as AbM (AbM is Oxford Molecular Ltd.'s antibody modelling package as described on www.bioinf.org.uk/abs/index.html), Chothia (Chothia and Lesk, 1987; Mol Biol. 196:901-17), Kabat (Kabat et al., 1991; 5th edition, NIH publication 91-3242), or IMGT (LeFranc, 2014; Frontiers in Immunology. 5 (22): 1-22). Said annotations further include delineation of CDRs and framework regions (FRs) in immunoglobulin-domain-containing proteins, and are known methods and systems to a skilled artisan who thus can apply these annotations onto any immunoglobulin protein sequences without undue burden. These annotations differ slightly, but each intend to comprise the regions of the loops involved in binding the target. The CDR region annotation for each VHH sequence described herein according to AbM is provided in Table 12. Alternatively, slightly different CDR annotations known in the art may be applied here to identify the CDR/FR regions of the ISVDs as disclosed herein and as indicated for instance for VHH7 and VHH8 in FIG. 21.

It should be noted that—as is well known in the art for VH domains and for VHH domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. The total number of amino acid residues in a VH domain and a VHH domain will usually be in the range of from 110 to 120, often between 112 and 115. It should however be noted that smaller and longer sequences may also be suitable for the purposes described herein.

In another embodiment the protein binding agent provided herein comprises an ISVD specifically binding the CI-M6PR extracellular N-terminal domains 1-3, wherein said ISVD contains a sequence selected from the group of sequences depicting the VHH1, 5, 7, 8, 1H11 or 1H52, as exemplified herein, as shown in SEQ ID NO:1,5,7,8, 24 and 25, resp., or a sequence with at least 85%, or at least 90%, or at least 95%, or at least 99% identity thereof, wherein the CDR regions are identical to the respective ISVD sequence, and variation of residues is solely present for non-binding residues of the FR regions.

A further embodiment relates to said protein binding agent comprising a CI-M6PR-specific ISVD comprising said CDRs of SEQ ID NO: 1, 5, 7, 8, 24 or 25, annotated according to AbM, as defined herein in Table 12, and comprising:
a FR1 sequence corresponding to any of the sequences included in the consensus sequence ‘xVQLxESGGGLVQxGGSLxLSCxAx’ (SEQ ID NO:78), wherein x at position 1 (x1) is Q, E, or D, x5 is Q or V, x14 is P or A, x19 is R or K, x23 is A, E, T, or V, and x25 is S or A;
a FR2 sequence corresponding to any of the sequences included in the consensus sequence ‘WxRQxPGKxxExVx’ (SEQ ID NO:79), wherein x at position 2 (x2) is L, F or Y, x5 is A or I, x9 is G, E, or Q, x10 is R or I, x12 is G, F, or W, x14 is S or A;
a FR3 sequence corresponding to any of the sequences included in the consensus sequence ‘YxDSxKxRFxxSRDxxKNTxxLxMNSLxxEDTAxxYCxx’ (SEQ ID NO:80), wherein x at position 2 (x2) is A, S, H, or D, x5 is V or A, x7 is G or D, x10 is S, T, or A, x11 is I or V, x15 is D or N, x16 is A, T, or S, x20 is L, I, or V, x21 is Y or N, x23 is R, Q, or Y, x28 is K, Q, or R, x29 is P or T, x34 is V or I, x35 is Y or V, x38 is K, A, or Y, x39 is A, R or C;
a FR4 sequence corresponding to any of the sequences included in the consensus sequence ‘xGQGTXVTVSS’ (SEQ ID NO:81), wherein x at position 1 (x1) is W or R, x6 is Q or L.

Said “x” residues as shown in the consensus FR sequences provide for the amino acid positions with possible variations without reducing the functionality of the ISVD, and for which the possible differences in identity are provided by said consensus sequences based on the sequences described for VHH1, 5, 7, 8, 1H11 and 1H52, and the humanization formats of VHH7 and VHH8 as disclosed in SEQ ID NO: 26-35. Moreover, in view of humanization for instance, even further substitutions of those amino acids at the respective positions will be possible without loss in effect, since amino acids of similar nature/type may be used as an alternative. For instance, substitutions may be allowed among aliphatic small amino acids (I, V, L), or among aromatic amino acids (F, W, Y, H), or among positively charged amino acids (K, R), or among negatively charged amino acids (D or E), or among small polar amino acids (S, T), or very small neutral amino acids (G, A).

More in particular, the FR1-4 regions of said CI-M6PR-specific ISVDs of the protein binding agents of the present invention can be provided by the FR sequences as provided in Table 13.

In a further embodiment, the protein binding agent as described herein comprises a CI-M6PR-specific ISVD selected from the group of SEQ ID NO:1, 5, 7, 8, 24 or 25, or a humanized variant of any one thereof. The term ‘humanized variant’ of an immunoglobulin single variable domain such as a domain antibody and Nanobody® (including VHH domain) refers to an amino acid sequence of said ISVD representing the outcome of being subjected to humanization, i.e. to increase the degree of sequence identity with the closest human germline sequence. In particular, humanized immunoglobulin single variable domains, such as Nanobody® (including VHH domains) may be immunoglobulin single variable domains in which at least one amino acid residue is present (and in particular, at least one framework residue) that is and/or that corresponds to a humanizing substitution (as defined further herein). Potentially useful humanizing substitutions can be ascertained by comparing the sequence of the framework regions of a naturally occurring VHH sequence with the corresponding framework sequence of one or more closely related human VH sequences, after which one or more of the potentially useful humanizing substitutions (or combinations thereof) thus determined can be introduced into said VHH sequence (in any manner known per se, as further described herein) and the resulting humanized VHH sequences can be tested for affinity for the target, for stability, for ease and level of expression, and/or for other desired properties. In this way, by means of a limited degree of trial and error, other or further suitable humanizing substitutions (or suitable combinations thereof) can be determined by the skilled person. Also, based on what is described before, (the framework regions of) an immunoglobulin single variable domain, such as a Nanobody® (including VHH domains) may be partially humanized or fully humanized. Humanized immunoglobulin single variable domains, in particular Nanobody, may have several advantages, such as a reduced immunogenicity, compared to the corresponding naturally occurring VHH domains. In summary, the humanizing substitutions should be chosen such that the resulting humanized amino acid sequence of the ISVD and/or VHH still retains the favourable properties, such as the antigen-binding capacity, and allosteric modulation capacity. The skilled person will be able to select humanizing substitutions or suitable combinations of humanizing substitutions which optimize or achieve a desired or suitable balance between the favourable properties provided by the humanizing substitutions on the one hand and the favourable properties of naturally occurring VHH domains on the other hand. Such methods are known by the skilled addressee. A human consensus sequence can be used as target sequence for humanization, but also other means are known in the art. One alternative includes a method wherein the skilled person aligns a number of human germline alleles, such as for instance but not limited to the alignment of IGHV3 alleles, to use said alignment for identification of residues suitable for humanization in the target sequence. Also, a subset of human germline alleles most homologous to the target sequence may be aligned as starting point to identify suitable humanisation residues. Alternatively, the VHH is analyzed to identify its closest homologue in the human alleles, and used for humanisation construct design. A humanisation technique applied to Camelidae VHHs may also be performed by a method comprising the replacement of specific amino acids, either alone or in combination. Said replacements may be selected based on what is known from literature, are from known humanization efforts, as well as from human consensus sequences compared to the natural VHH sequences, or the human alleles most similar to the VHH sequence of interest. As can be seen from the data on the VHH entropy and VHH variability given in Tables A-5-A-8 of WO 08/020079, some amino acid residues (i.e. hallmark residues) in the framework regions are more conserved between human and Camelidae than others. Generally, although the invention in its broadest sense is not limited thereto, any substitutions, deletions or insertions are preferably made at positions that are less conserved. Also, generally, amino acid substitutions are preferred over amino acid deletions or insertions. For instance, a human-like class of Camelidae single domain antibodies contain the hydrophobic FR2 residues typically found in conventional antibodies of human origin or from other species, but compensating this loss in hydrophilicity by other substitutions at position 103 that substitutes the conserved tryptophan residue present in VH from double-chain antibodies. As such, peptides belonging to these two classes show a high amino acid sequence homology to human VH framework regions and said peptides might be administered to a human directly without expectation of an unwanted immune response therefrom, and without the burden of further humanisation. Indeed, some Camelidae VHH sequences display a high sequence homology to human VH framework regions and therefore said VHH might be administered to patients directly without expectation of an immune response therefrom, and without the additional burden of humanization. Suitable mutations, in particular substitutions, can be introduced during humanization to generate a polypeptide with reduced binding to pre-existing antibodies (reference is made for example to WO 2012/175741 and WO2015/173325), for example in at least one of the positions: 11, 13, 14, 15, 40, 41, 42, 82, 82a, 82b, 83, 84, 85, 87, 88, 89, 103, or 108. The amino acid sequences and/or VHH of the invention may be suitably humanized at any framework residue(s), such as at one or more Hallmark residues (as defined herein) or preferably at one or more other framework residues (i.e. non-Hallmark residues) or any suitable combination thereof. Depending on the host organism used to express the amino acid sequence, ISVD, VHH or polypeptide of the invention, such deletions and/or substitutions may also be designed in such a way that one or more sites for posttranslational modification (such as one or more glycosylation sites at asparagine to be replaced with G, A, or S; and/or Methionine oxidation sites) are removed, as will be within the ability of the person skilled in the art. Alternatively, substitutions or insertions may be designed so as to introduce one or more sites for attachment of functional groups, for example to allow site-specific pegylation. In some cases, at least one of the typical Camelidae hallmark residues with hydrophilic characteristics at position 37, 44, 45 and/or 47 is replaced (Kabat No; see WO2008/020079 Table A-03). Another example of humanization includes substitution of residues in FR 1, such as position 1, 5, 11, 14, 16, and/or 23, and/or 28; in FR2 such as positions 40 and/or 43; in FR3, such as positions 60-64, 73, 74, 75, 76, 78, 79, 81, 82b, 83, 84, 85, 93 and/or 94; and in FR4, such as position 103, 104, 105, 108 and/or 111 (see WO2008/020079 Tables A-05-A08; all numbering according to the Kabat).

In a specific embodiment, the protein binding agent as described herein comprises a CI-M6PR-specific ISVD comprising a humanized variant of VHH7 or VHH8, which corresponds to any one of SEQ ID NOS: 26-35, for which retained functionality was shown in Callewaert et al. (PCT/EP2022/054278).

Another embodiment relates to a protein binding agent comprising an ISVD specifically binding to CI-M6PR domain 1-3, as described herein, and a binding agent specifically binding an extracellularly-accessible target, which is a multi-specific agent, further comprising a binding agent or moiety directly or indirectly linked or coupled to any said CI-M6PR-specific ISVD or target-specific binding agent, with specificity for a different epitope and/or different target. Said further binding agent or moiety may thus comprise a binding agent specific for a CI-M6PR, but with a chemical structure different from the first binding agent, this may result in a multiparatopic or multispecific binding agent, or said further binding agent may comprise a binding agent specific for binding the same extracellularly-accessible target as the binding agent of the fusion protein, but binding to another epitope on said target, or may bind another extracellularly-accessible target. Moreover, said further binding agent may specifically bind another target that is capable of extending the fusion's protein half-life in a subject, such as for instance serum albumin protein. Said further binding agent may thus comprise an antigen-binding domain, and/or may be a functional moiety. When said further binding agent comprises a binding agent with the same or identical in structure or sequence as compared to the other building blocks of the fusion protein, i.e. the CI-M6PR-specific and extracellularly-accessible-target-specific binders, this provides for a multivalent binder for any of said respective binders, which may increase the avidity for binding for instance. Furthermore, said further binding agent may also comprise another form of a CI-M6PR binding agent, including a binding agent with a different target specificity, or binding a different lysosomal-targeting protein. By coupling several binders, which all may comprise an ISVD in a specific embodiment, interacting with different targets, preferably targets present on the cell surface or extracellular environment, these are defined as multispecific binding agents. In a specific embodiment, the fusion protein comprises more than one VHH as disclosed herein to specifically interact with the CI-M6PR and a binding agent for the extracellularly-accessible target. Another specific embodiment relates to a fusion protein comprising one binding agent specific for CI-M6PR and a multivalent or multispecific binding agent for the extracellularly-accessible target protein of interest. In the specific embodiments where several ISVDs are used as binding agents, a “multi-specific” form for instance, is formed by bonding together two or more immunoglobulin single variable domains, of which at least one with a different specificity.

So, the invention relates to bifunctional bispecific agents which target CI-M6PR, as described herein, and as a second binding specifically target a cell surface molecule or extracellular molecule, i.e. an extracellularly-accessible protein (different from the CI-M6PR protein) wherein such a bispecific agent may enhance degradation of the target relative to degradation of the cell surface molecule or extracellular molecule in the presence of the CI-M6PR binding agent alone (so not coupled to said further binding agent specifically binding the target). The protein binding agent of the present invention is in itself already bispecific in nature, as it binds at least CI-M6PR and another extracellularly-accessible protein. So multispecific binding agents or fusion proteins may also relate to the addition of a further binding agent, which may bind one of the same or further targets. Non-limiting examples of multi-specific constructs include “bi-specific” constructs, “tri-specific” constructs, “tetra-specific” constructs, and so on. To illustrate this further, any multivalent or multi-specific (as defined herein) protein binding agent of the invention may be suitably directed against two or more different epitopes on the same antigen, for example against epitope 1 on one domain and epitope 2 on another domain of CI-M6PR; or may be directed against two or more different antigens, for example against CI-M6PR and one as a half-life extension against Serum Albumin. One of the most widely used techniques for increasing the half-life and/or reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). Another technique for increasing the half-life of a binding domain may comprise the engineering into bifunctional or bispecific domains (for example, one or more ISVDs or active antibody fragments against CI-M6PR coupled to one ISVD or active antibody fragment against serum albumin aiding in prolonging half-life)) or into fusions of antibody fragments, in particular immunoglobulin single variable domains, with peptides (for example, a peptide against a serum protein such as albumin). The coupling to additional moieties will result in multispecific binding agent, as further disclosed herein.

Multivalent or multi-specific binding agents of the invention may also have (or be engineered and/or selected for) increased avidity and/or improved selectivity for the desired CI-M6PR interaction, and lysosome targeting function, and/or for any other desired property or combination of desired properties that may be obtained by the use of such multivalent or multi-specific binding agents. For instance, the combination of one or more ISVDs binding any of the CI-M6PR epitopes, and one or more ISVDs binding any of an extracellularly-accessible target epitope as described herein, results in a multi-specific binding agent of the invention with the potential of cellular uptake or internalisation of the full complex of protein binding agent and its targets bound to it, via CI-M6PR internalisation, which may ultimately lead to degradation of said target(s) in the lysosome. With “internalisation” of the extracellularly-accessible target protein is meant herein that the target is removed from the cell surface to an extent that is higher when bound to the protein binding agent of the present invention (thus including the CI-M6PR-ISVD), as compared to a control, which may be the same protein binding agent without said CI-M6PR-ISVD or with an alternative ISVD that does not specifically bind the CI-M6PR or other target for lysosomal uptake; and internalisation can also be expressed as the voxel counts/minute (as determined in a life cell imaging method and as herein considered as ‘internalised’ with an internalisation rate of at least 15 voxel counts/min, or at least 35, or at least 50, or at least 65, or at least 80, or at least 100, or at least 120 voxel counts/minute). With “degradation” or “enhanced degradation” as compared to a control is meant herein that the protein quantity of said target is reduced, when determined for total protein (including the cell-surface retained protein fraction), or when the intracellular fraction or lysate of the cells after internalisation of said target protein qualitatively indicates protein degraded into several fragments (as for instance determined by Western blot analysis, as exemplified herein). With ‘degradation relative to a control’ (e.g. untreated or treatment with a comparable protein binding agent lacking the CI-M6PR-specific ISVD of the protein biding agent of the present invention), is meant that the protein level is reduced with at least 5%, at least 10%, at least 15%, at least 20%, at least 30%, at least 50%, or more, as compared to the control (and preferably based on normalized protein levels using a control protein for normalisation).

In a further embodiment, the protein binding agent of the present invention is a multi-specific binding agent which comprises at least said a CI-M6PR-specific ISVD as described herein, and an extracellularly-accessible target protein-specific binding agent, which may be coupled via a linker, spacer. Upon binding CI-M6PR, said multi-specific binding agent or multivalent ISVD may have an additive or synergistic impact on the CI-M6PR internalizing activity, or may be used to target and extract or shuffle cell-surface or extracellular molecules from the extracellular or membrane environment into the endosomes and lysosome, or alternatively, used to prolong their half-life by recycling those targets through the endosome cycling pathway. The multispecific binders of the invention may be coupled to a functional moiety, a therapeutic (further targeting) moiety, a half-life extending moiety, or to a cell penetrant carrier.

In a further specific embodiment, said extracellularly-accessible target protein-specific binding agent may comprise an antigen-binding domain, such as an ISVD, a VHH, a Nb, a VHH-Fc fusion, a VHH-Fc-VHH fusion, a knob-into hole VHH-Fc fusion or an antibody, such as an IgG, or alternatively may comprise a small molecule (which may be linked via covalent chemical coupling) or may be a peptide or peptidomimetic. Further specific embodiments relate to bispecific or multispecific formats comprising said ISVD-based CI-M6PR binders as described herein, and directly or indirectly via a spacer or linker, or chemically, coupled to extracellularly-accessible target protein-specific and optionally further binding agents. Said coupling or fusion of a CI-M6PR specific ISVD to for instance, another ISVD, antibody fragment or antibody-type of VH or VL structure as defined herein, may also occur through linking via an Fc tail as to produce bispecific ISVD-Fc antibodies, as discussed above.

Hence, specific embodiments envisaged herein include the those bispecific chimeras, wherein the ISVD-based binder specifically interacting with the N-terminal part of CI-M6PR retains its binding to the CI-M6PR during its endosomal cycle, and this has a binding affinity that is stable and resistant to dissociation down to pH˜5.5. The anti-CI-M6PR VHHs described herein provide for a panel of highly specific and high affinity binders at neutral pH, though with different pH dissociation profiles when lowering pH (in vitro) down to pH6, 5, 4.5 or 4. This panel thus provides for a versatile toolbox to explore bispecifics with lysosomal degradation and recycling potential of different nature depending on the needs for specific targets and applications. Moreover, the high affinity of said CI-M6PR binding agents (nanomolar to picomolar K_{D} values) at neutral pH is required as to ensure specific tight binding to the receptor on the cell surface, though subsequently a need to dissociate rapidly when internalized in endosome/lysosome may be desired as to increase the chance that the same late endosomal/lysosomal delivery route is followed as the natural cargo of the CI-M6PR. In view of optimizing binding affinity at specific pH conditions, methods are known to the skilled person as how to engineer the binding agents such as the VHHs using for instance histidine scanning method mutagenesis [22], which is specifically aimed at reducing the binding affinity of antibodies at acidic pH as compared to neutral pH. As the imidazole side chain of a histidine residue has a pKa˜6.0, the switching of its protonation state alters binding interactions at interfaces where it occurs. Briefly, a combinatorial phage library is obtained with histidines incorporated into the VHH CDRs. This library will then be screened through biopanning with binding at pH 7.4 and elution at pH 5.5, followed by determination of the exact binding characteristics of the resulting VHHs at these pH's through BLI.

Another specific embodiment relates to protein binding agents comprising the CI-M6PR-specific ISVD, as described herein, and a binding agent for another extracellularly-accessible protein, which is fused or coupled by a genetic fusion, and produced through recombinant expression in a host.

Another aspect of the invention provides for a method for detecting the presence, absence or level of CI-M6PR and/or extracellularly-accessible target protein in a sample, the method comprising: contacting the sample with the protein binding agent as described herein, and detecting the presence or absence or level, i.e. quantifying, the bound CI-M6PR ISVD, or target protein binding agent, which is optionally a labelled, conjugated or multispecific binding agent. The sample used herein may be a sample isolated from the body, such as a body fluid, including blood, serum, cerebrospinal fluid, among others, or may be an extract, such as a protein extract, a cell lysate, etc.

For the purpose of detection and/or imaging, in vitro or in vivo, the protein binding agent or fusion protein of the invention, comprising a CI-M6PR-specific ISVD and a binding agent specifically binding the extracellularly-accessible protein, as described herein may further comprise in some embodiments a detection agent, such as a tag or a label. For instance, the ISVDs, VHHs, or Nbs as exemplified herein were also tagged. Such a tag allows affinity purification and detection of the antibody or active antibody fragments of the invention.

Some embodiments comprise the protein binding agent, further comprising a label or tag, or more specifically, the fusion protein labelled with a detectable marker. The term detectable label or tag, as used herein, refers to detectable labels or tags allowing the detection and/or quantification of the fusion protein as described herein, and is meant to include any labels/tags known in the art for these purposes. Particularly preferred, but not limiting, are affinity tags, such as chitin binding protein (CBP), maltose binding protein (MBP), glutathione-S-transferase (GST), poly(His) (e.g., 6× His or His6), biotin or streptavidin, such as Strep-Tag®, Strep-tag II® and Twin-Strep-Tag®; solubilizing tags, such as thioredoxin (TRX), poly (NANP) and SUMO; chromatography tags, such as a FLAG-tag; epitope tags, such as V5-tag, myc-tag and HA-tag; fluorescent labels or tags (i.e., fluorochromes/-phores), such as fluorescent proteins (e.g., GFP, YFP, RFP etc.) and fluorescent dyes (e.g., FITC, TRITC, coumarin and cyanine); luminescent labels or tags, such as luciferase, bioluminescent or chemiluminescent compounds (such as luminal, isoluminol, theromatic acridinium ester, imidazole, acridinium salts, oxalate ester, dioxetane or GFP and its analogs); phosphorescent labels; a metal chelator; and (other) enzymatic labels (e.g., peroxidase, alkaline phosphatase, beta-galactosidase, urease or glucose oxidase); radioisotopes. Also included are combinations of any of the foregoing labels or tags. Technologies for generating labelled polypeptides and proteins are well known in the art. A protein binding agent or fusion protein as described herein comprising a CI-M6PR-specific ISVD of the invention, and a binding agent for an extracellularly-accessible target, coupled to, or further comprising a label or tag allows for instance immune-based detection of said bound fusion protein. Immune-based detection is well known in the art and can be achieved through the application of numerous approaches. These methods are generally based upon the detection of a label or marker, such as described above. See, for example, U.S. Pat. Nos. 3,817,837; 3,850,752; 3,939,350; 3,996,345; 4,277,437; 4,275,149 and 4,366,241. In the case where multiple antibodies are reacted with a single array, each antibody can be labelled with a distinct label or tag for simultaneous detection. Yet another embodiment may comprise the introduction of one or more detectable labels or other signal-generating groups or moieties, or tags, depending on the intended use of the labelled or tagged fusion protein of the present invention. Other suitable labels will be clear to the skilled person, and for example include moieties that can be detected using NMR or ESR spectroscopy. Such labelled fusion protein, such as those as described herein may for example be used for in vitro, in vivo or in situ assays (including immunoassays known per se such as ELISA, RIA, EIA and other “sandwich assays”, etc.) as well as in vivo imaging purposes, depending on the choice of the specific label.

Another aspect of the invention relates to a pharmaceutical composition comprising the protein binding agent or fusion protein of the invention, as described herein, or comprising the nucleic acid molecule, or vector as described herein, and optionally a pharmaceutically acceptable carrier or diluent or excipient. These pharmaceutical compositions can be utilized to achieve the desired pharmacological effect by administration to a patient in need thereof.

A further aspect relates to said protein binding agent of the invention, comprising an ISVD-based CI-M6PR-specific fusion protein further recognizing an extracellularly-accessible target protein for internalization and degradation, the nucleic acid molecule or the vector encoding said protein binding agent or fusion protein, or the pharmaceutical composition comprising these, as described herein, for use as a diagnostic.

In a particular embodiment, kits are provided which contain means to degrade the extracellularly-accessible target protein, said kit including the protein binding agent as described herein, allowing to detect or modulate trafficking of a target protein in a system, which may be an in vitro or in vivo system. It is envisaged that these kits are provided for a particular purpose, such as for endosome/lysosome labelling, or to trigger target protein degradation in vitro, or for in vivo imaging, or for diagnosis of an altered CI-M6PR or target quantity, response or effect in a subject. In another embodiment, said kit is provided which contains means including a nucleic acid molecule, a vector, or a pharmaceutical composition as described herein. The means further provided by the kit will depend on the methodology used in the application, and on the purpose of the kit. For instance, detection of a labelled fusion protein, as described herein, or nucleic acid molecule as described herein, which may be desired for CI-M6PR or target quantification on nucleic acid or protein level. For protein-based detection, the kits typically will contain labelled or coupled binding agents such as ISVDs. Likewise, for detection at the nucleic acid level, the kits may contain labels for nucleic acids such as primers or probes. Further control agents, antibodies or nucleic acids may also be provided in the kit. A standard, for reference or comparison, a CI-M6PR or target protein substrate or signaling component, a reporter gene or protein or other means for using the kit may also be included. Of course, the kit may further comprise pharmaceutically acceptable excipients, buffers, vehicles or delivery means, an instruction manual and so on.

A specific aspect of the invention relates to a protein binding agent comprising an ISVD-based CI-M6PR binding agent, as described herein, and a binding agent specifically binding the epidermal growth factor receptor (EGFR) extracellular-accessible target protein, which is located at the cell surface as transmembrane receptor protein. In addition to the proof of concept experiments exemplified herein showing that an extracellularly added protein, such as GFP, can be effectively internalized and degraded in the endosomal/lysosomal machinery, the proof of concept for internalisation and degradation of a transmembrane protein was provided by using a protein binding agent wherein the EGFR-binding agent was provided by a Nb or an antibody, in combination with the coupled VHH7 or VHH8 CI-M6PR-specific ISVD. EGFR targeting for CI-M6PR-mediated internalisation and preferably also lysosomal degradation thereby provides for an alternative approach in therapeutic treatment of several cancers. Said binding agent targeting or specifically binding EGFR may be envisaged herein as any type of binding agent that can be fused to said CI-M6PR-specific ISVD, so the EGFR-specific binding agent may be an antibody, a small molecule, a peptide, or another antigen-binding protein, including an ISVD or VHH or Nb. In a specific embodiment said EGFR-specific binding agent comprises a Nb or a functional (mutant) variant thereof, including for instance but not limited to monovalent 9G8 VHH as presented in SEQ ID NO:12, or a functional homologue with at least 80%, 85%, 90%, 95%, or 97% or 99% identity thereof taken over the total length of the monovalent ISVD. With a functional homologue is meant that the binding properties of said ISVD homologue remain very similar or the same, as defined herein. Preferably the amino acid residues in the CDRs are identical in said functional homologues, unless when a mutation does not affect binding properties significantly and/or another hurdle such as glycosylation can be avoided by introducing such a mutation in the CDRs, as for examples for the mutant variant of SEQ ID NO: 12 provided in SEQ ID NO:17, wherein the N-glycosylation on the Serine was avoided by an S54A substitution (according to Kabat numbering), or any functional homologue with at least 80%, 85%, 90%, 95%, or 97% or 99% identity thereof taken over the total length of the monovalent ISVD. When fused to said CI-M6PR-specific VHHs, this results in the bispecific fusion proteins as exemplified herein in SEQ ID NO: 13, 18, 82, or 84, or a functional homologue with at least 80%, 85%, 90%, 95%, or 97% or 99% identity thereof taken over the total length of each monovalent ISVD, and/or of the total length of the fusion protein. Specifically said EGFR-binding agent fused to said CI-M6PR-specific ISVD may be a multivalent or multispecific EGFR-specific binding agent, more specifically may comprise SEQ ID NO: 14, SEQ ID NO: 19, SEQ ID NO: 85, or a functional homologue with at least 80%, 85%, 90%, 95%, or 97% or 99% identity thereof taken over the total length of each monovalent ISVD, and/or of the total length of the fusion protein. In a further specific embodiment said EGFR-specific binding agent is a conventional antibody, herein provided as cetuximab, provided by the combination of its heavy chain as in SEQ ID NO: 87 and the light chain as presented in SEQ ID NO: 86, or as exemplified herein, said protein binding agent may be provide as the heavy chain of SEQ ID NO: 87 being fused to said CI-M6PR-specific ISVD described herein, such as provided in SEQ ID Nos: 88 or 89, to result in a multispecific EGFR-specific binding agent, which in combination with the light chain provided by SEQ ID NO: 86 is capable of internalizing and degrading EGFR in a potent manner. Said protein binding agents specifically targeting EGFR may thus be used as a medicine, more specifically for use in treatment of cancer.

It is to be understood that although particular embodiments, specific configurations as well as materials and/or molecules, have been discussed herein for products, compositions, methods, uses, samples and biomarker products according to the disclosure, various changes or modifications in form and detail may be made without departing from the scope of this invention. The following examples are provided to better illustrate particular embodiments, and they should not be considered limiting the application. The application is limited only by the claims.

### EXAMPLES

The examples described below show for the first time the development of a Nanobody-based LYTAC, providing proof of concept for a novel bi-/multi-specific platform that couples an anti-CI-M6PR VHH to an antigen-binding protein, such as a VHH or antibody, against an extracellularly-accessible protein of interest, as to target this protein for internalisation and/or lysosomal degradation. The efficacy of internalisation of such multispecific fusion proteins is shown herein in a proof of concept experiment using GFP as extracellular target protein and a GFP-specific VHH, and further explored in the context of transmembrane proteins, such as relevant disease-causing proteins, herein demonstrated using the human epidermal growth factor receptor (EGFR) as a target, with binding agents specifically targeting said receptor fused to anti-CI-MPR VHHs, which we previously isolated after a llama immunization and screening campaign. Since Hela cells are commonly applied as general cell-based model for cancer, we explored whether our Nb-based LYTAC-system could induce internalisation and/or degradation of EGFR on those cells.

### Example 1. Production and Purification of VHH-Based Anti-EGFR LYTAC Constructs

Anti-CI-M6PR VHHs have previously been generated and characterized in the context of enzyme replacement therapy for lysosomal storage diseases (Callewaert et al., PCT/EP2022/054278). In short, alpacas were immunized with domains 1-3 of the human CI-M6PR and a series of phage pannings according to the method in yielded a number of human/mouse cross-reactive VHHs. The VHHs with the most similar affinity between human and mouse were tested and shown to trigger CI-M6PR-mediated lysosomal uptake. As discussed in PCT/EP2022/054278, further characterization has revealed that a selected panel of VHHs, as disclosed and used herein, is suitable for cell-expressed M6PR binding and internalization (Example 4). Said panel could further be grouped into VHHs specific for binding to two epitopes present on the N-terminal M6PR extracellular region (Example 6). Out of those, we selected VHH8 in this example, which binds with high affinity at the pH of plasma (7.4) (K_{D}˜3.35E10-9), as assessed by biolayer interferometry (BLI), for incorporation in the initial nanoLYTAC constructs. As a negative control for internalization/degradation that is not mediated by the CI-M6PR, a GFP-binding VHH (GBP) was used.

As it has been shown that bivalent binding of the CI-M6PR increases the rate of internalization through receptor dimerization [7], both a CI-M6PR monovalent and bivalent format of each construct may be analyzed for assessment of degradation potency and efficacy. To create bivalency, two CI-M6PR VHHs may be linked with for instance a standard Gly4 Ser linker.

In first instance, the monovalent VHH8 has been incorporated in a set of anti-EGFR LYTAC constructs together with an extracellularly-accessible target protein-specific VHH, more specifically for the target chosen herein being EGFR. A VHH (called PMP9G8, hereafter referred to as 9G8; SEQ ID NO:12) that binds to EGFR [5-6] with monovalent affinity in the low nanomolar range and inhibits EGFR-mediated signaling, as has been described, is used for incorporation in our anti-EGFR nanoLYTACs. As receptor tyrosine kinases can forms homodimers upon activation, bivalent binding of EGFR might improve the apparent affinity, resulting in a more potent and efficacious CI-M6PR-mediated internalization. Moreover, it has also been shown that dimerization of EGFR by itself can drive receptor internalization and subsequent downregulation by degradation in the lysosome [8]. To account for this, a fusion construct of the GBP-VHH connected to two anti-EGFR VHHs in tandem (bivalent format) is envisaged herein.

The constructs used herein were cloned using a modular cloning platform, produced in wild type Komagataella phaffii, hereafter named Pichia pastoris, followed by purification, after which their EGFR-internalization and -degradation potential was investigated on Hela cells.

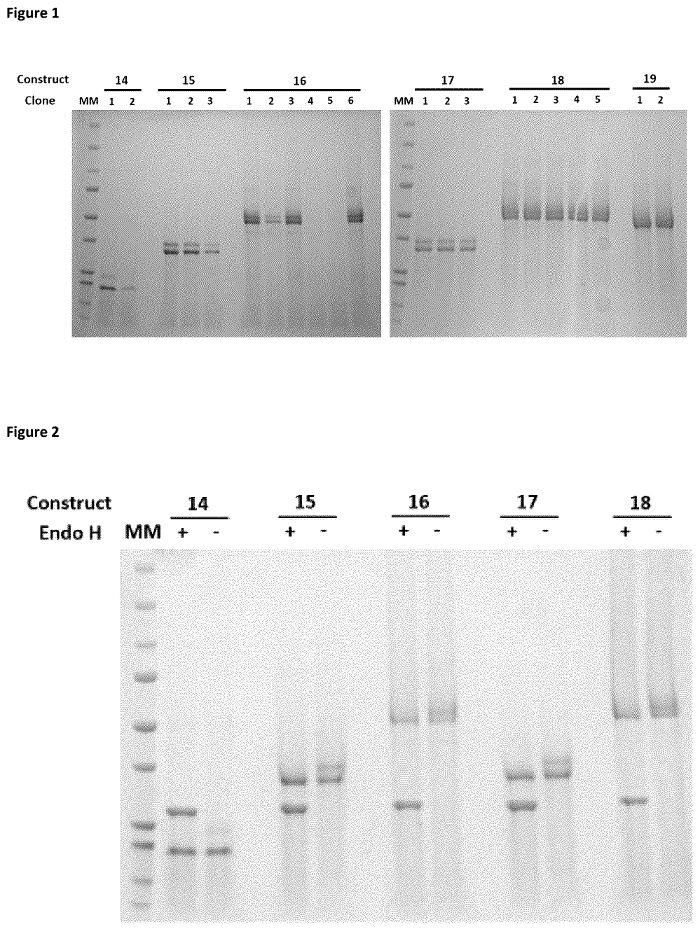

Table 1 summarizes the set of LYTAC constructs that was initially cloned and expressed in P. pastoris. SDS-PAGE analysis of expression tests demonstrated two distinct bands, one at the expected molecular weight and one corresponding to a larger MW (FIG. 1).

TABLE 1
Summary of the constructs.

| Construct | VHH fusion | Anti-EGFR | | Anti-CI- |
|---|---|---|---|---|
| Nr. | SEQ ID NO: | VHH | Linker | M6PR VHH |
| 14 | 12 | 9G8 | / | / |
| 15 | 13 | 9G8 | G4Sx3 | VHH8 |
| 16 | 14 | 9G8 + G4Sx3 + 9G8 | G4Sx3 | VHH8 |
| 17 | 15 | 9G8 | G4Sx3 | GBP |
| 18 | 16 | 9G8 + G4Sx3 + 9G8 | G4Sx3 | GBP |
| 25 | 17 | 9G8 S54A | / | / |
| 26 | 18 | 9G8 S54A | G4Sx3 | VHH8 |
| 27 | 19 | 9G8 S54A + G4Sx3 + | G4Sx3 | VHH8 |
| | | 9G8 S54A | | |
| 28 | 20 | 9G8 S54A | G4Sx3 | GBP |
| 29 | 21 | 9G8 S54A + G4Sx3 + | G4Sx3 | GBP |
| | | 9G8 S54A | | |

9G8: EGFR-specific VHH PMP9G8; G4S: Gly-Ser linker; S54A: Ser to Ala mutation in 9G8 VHH at position 54 (according to Kabat numbering); VHH8: CI-M6PR-specific VHH (SEQ ID NO: 8); GBP: GFP-binding VHH (negative control).
In addition to the SEQ ID VHH amino acid sequence, each construct contains an N-terminal RSM triple amino acid sequence (due to a cloning error).

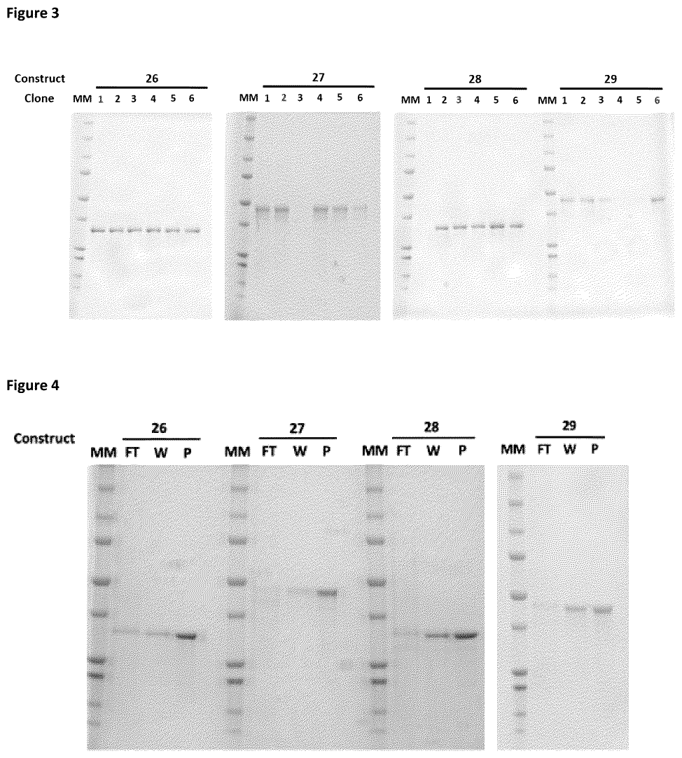

Analysis of an endoglycosidase H (EndoH)-digestion of the supernatant indicated N-glycosylation of the 9G8 anti-EGFR VHH in P. pastoris (FIG. 2). Indeed, in the amino acid sequence of 9G8, an N-W-S sequon was identified in the CDR2 region. Analysis of the existing crystal structures of 9G8 in complex with the EGFR ectodomain revealed that none of the residues from the CDR2 region are involved in antigen binding. In an attempt to remove the N-glycosylation, a new set of LYTAC constructs was produced in which 9G8 contained a S54A mutation (Table 1). Expression tests confirmed the removal of glycosylation, demonstrated by the absence of the additional band on SDS-PAGE (FIG. 3). For each construct, a suitable clone was selected and larger-scale expression and purification was performed through benchtop gravity flow IMAC and desalting (FIG. 4).

Furthermore, in order to achieve the highest degradation efficacy, it is envisaged to generate a fusion of the CI-M6PR VHH to the anti-EGFR VHH containing a protease-sensitive linker that has a cathepsin-cleavage site to assure that the target is released in the late endosome. Between two identical VHHs, a standard flexible 15 amino acid Gly4Ser linker may therefore be used.

Finally, although VHHs have a lot of advantages for therapeutic applications, a downside is their rapid renal clearance upon intravenous administration [9], which is somewhat reduced but still rapid for VHH concatemers. It is thus expectedly beneficial to improve the pharmacokinetic properties of the anti-EGFR nanoLYTACs, for intravenous injection in in vivo studies. Hence, also further variants of these nanoLYTAC constructs are envisaged herein wherein the binders are fused to an anti-albumin VHH. As albumin is constantly recycled by the neonatal Fc-receptor (FcRn) on vascular endothelial cells, it is rescued from glomerular filtration, resulting in a half-life of 19-21 days in humans [12]. In addition, this fusion further increases the hydrodynamic radius of the nanoLYTAC constructs.

### Example 2. In Vitro Assays for Evaluation of LYTAC-Mediated EGFR-Internalisation Efficacy by Flow Cytometry

The disappearance of the target protein from the cell surface can be quantified through flow cytometry. A decrease in the signal of the antibody-coupled fluorescence staining intensity would be detected upon internalization of the target protein.

By detecting cell-surface EGFR using flow cytometry, the EGFR-internalizing efficacy of the first set of EGFR-LYTAC constructs after HeLa cell-treatment was evaluated. Hela cells were either left untreated or were incubated with 5 or 50 nM of construct 26 (9G8 S54A-VHH8) or 27 (2x9G8 S54A-VHH8) or the corresponding control construct 28 (9G8 S54A-GBP) or 29 (2x9G8 S54A-GBP) in duplicate for 24 hours. After the incubation period, the cells were harvested and stained specifically for EGFR.

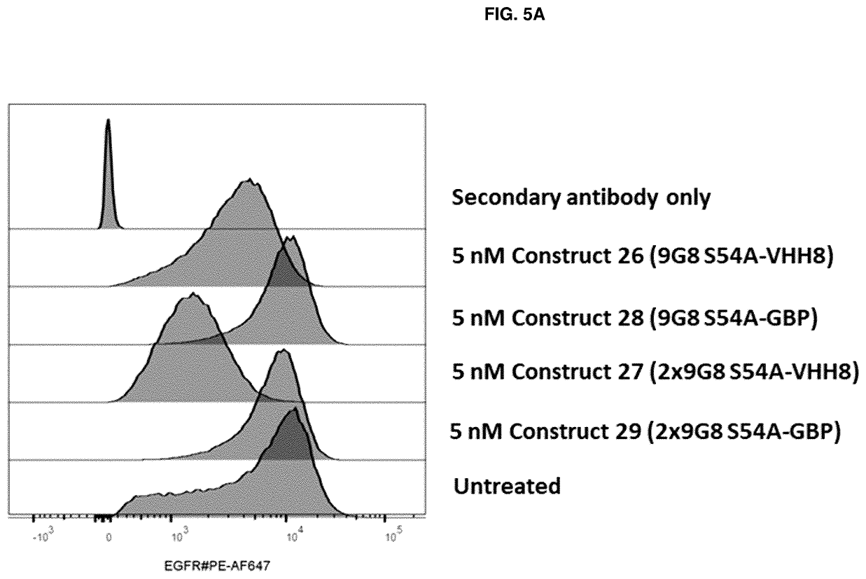

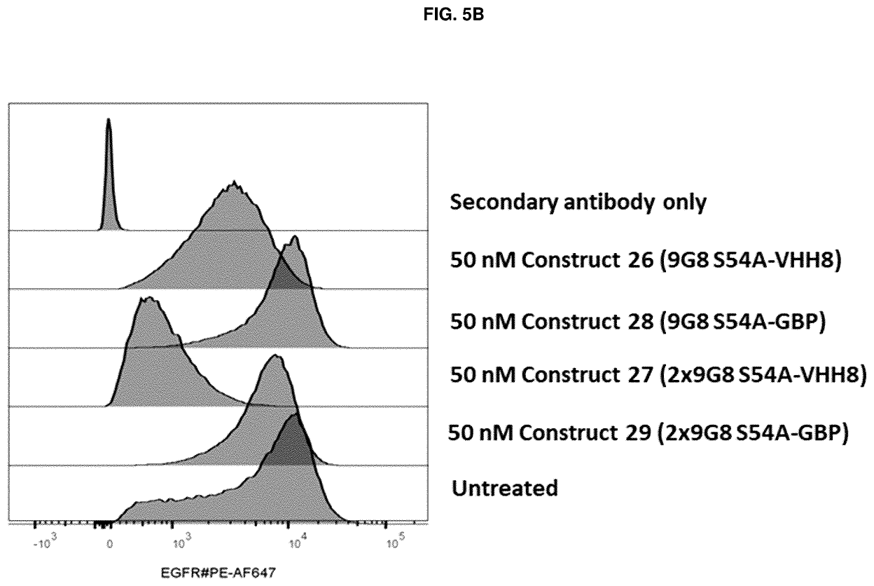

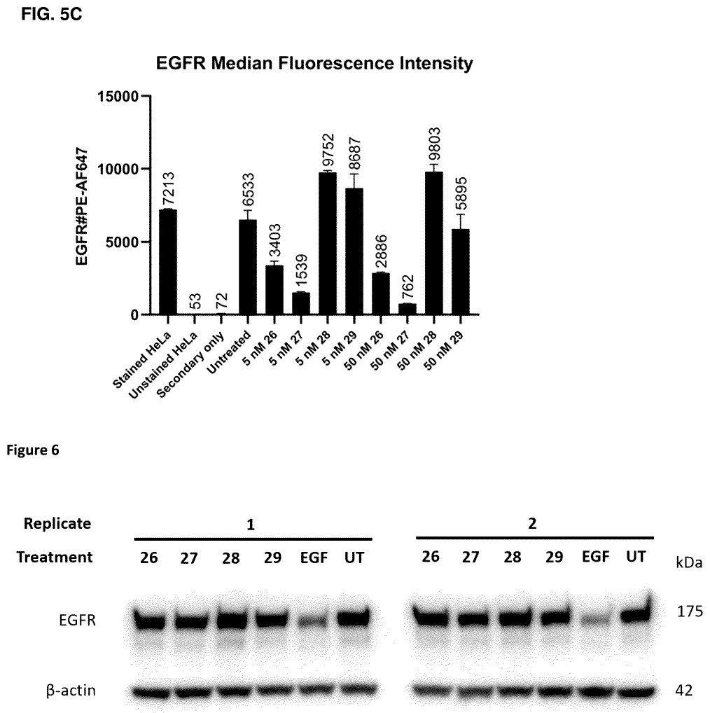

In FIG. 5A-B representative histograms of the fluorescent signal corresponding to cell-surface EGFR were set out for untreated Hela cells and for Hela cells treated with LYTAC-constructs (26 and 27) or with the corresponding control constructs (28 and 29 respectively). In FIG. 5C, a bar chart is shown indicating the median fluorescence intensity values measured for each condition in the experiment. The results demonstrate that the signal for cell-surface EGFR was reduced for cells treated with the LYTAC constructs 26 and 27 in comparison to the untreated cells or for cells treated with the corresponding control constructs 28 and 29. Furthermore, the bivalent EGFR-binding LYTAC (construct 27) resulted in a lower signal as compared to treatment with the monovalent EGFR-binding LYTAC (construct 26) at both the 5 and 50 nM concentrations, with the highest effect for both constructs being observed at 50 nM (FIG. 5B). The peak of the EGFR-signal of cells treated with construct 29 was slightly shifted to the left as compared to the peak of the untreated cells, indicating a modest VHH8-independent internalization-effect most likely due to bivalent binding of EGFR.

### Example 3. In Vitro Assays for Evaluation of LYTAC-Mediated EGFR-Degradation by Western Blot

As to quantify what fraction of the target protein is degraded in response to treatment with the nanoLYTAC, a chemiluminescent Western Blot analysis was performed. Hela cells were incubated with 50 nM of construct 26 (9G8 S54A-VHH8) or 27 (2x9G8 S54A-VHH8) or the corresponding control construct 28 (9G8 S54A-GBP) or 29 (2x9G8 S54A-GBP) during 24 hours in OptiMEM (in duplicate). Lysates were obtained and subjected to immunoblotting with an anti-EGFR antibody (FIG. 6). As a positive control, cells were incubated with EGF, which is known to induce EGFR degradation. In the described Western Blot experiment, we were not effectively able to detect a lower level of total EGFR for the cells treated with the said nanoLYTAC constructs as compared to the untreated cells or the cells treated with the control constructs. However, we speculate that this may be a cause of the current experimental set-up and so optimization of the conditions and methods is required, and is further detailed in Examples 10 & 12.

### Example 4. Anti-CI-M6PR VHH1, VHH5, VHH7 and VHH8 are Endocytosed and Colocalise with Late Endosome and Lysosomes

To demonstrate that the selected panel of M6PR-binding VHHs effectively internalizes and traffics through the endosomal compartments to the lysosome, several experiments were performed previously, as described in Callewaert et al. (PCT/EP2022/054278), and as summarized herein.

Targeting to the endolysosomal compartment was assessed by monitoring AAF488-VHH signal inside living cells over time (3h). Three fields of view were imaged every 6 minutes after LTR-incubation and administration of AF488-labeled anti-CI-M6PR VHHs, GFP binding protein (GBP) and recombinant human acid glucosidase a (rhGAA; positive control). After imaging, we calculated the uptake per cell volume by dividing the sum of voxel count for each fluorescent VHH by the sum of voxel count per imaged cell at a certain time point.

The uptake of the proteins relative to cell volume provided the best result for VHH1, -5, -7 and -8. The highest uptake of protein relative to cell volume was observed for VHH7, following a sigmoidal trend observed over three hours and an internalisation rate of 125.5×10⁴ summed AF-voxels/minute. Similarly, calculated by dividing the sum of AF488-positive voxel counts by time, the internalisation rate for VHH1 was 138.2×10⁴ summed AF-voxels/minute. Compared to VHH7 and -1, the observed intracellular fluorescence of VHH5 was lower and more variable, while for VHH8 and rhGAA, internalisation rates were 68.7, 67.3 and 17.8×10⁴ summed AF-voxels/minute The profiles of the remaining VHHs (VHHs 1-11 were analyzed herein) were comparable to the negative control (GBP) and confirm that these indeed do not bind cell-surface hCI-M6PR.

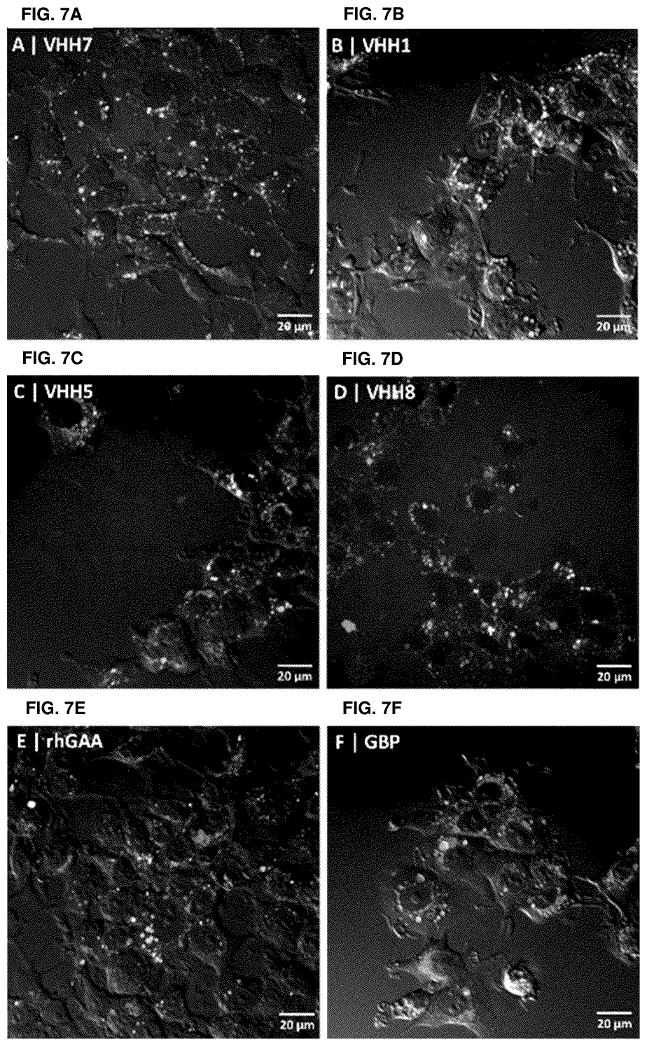

The mean percentages of VHH colocalising with lysosomes were calculated by taking the ratio of the voxel counts of intracellular AF488-signal that colocalized with LTR and of the total intracellular VHH signal. Next to this also the mean percentage of the entire endolysosomal pool containing the particular VHH or rhGAA was determined by the voxel count ratio of the VHH-signal colocalising with LTR and the total LTR signal. Due to the—sometimes—low intracellular AF488 signal and variable percentages, the absolute voxel counts of the intracellular VHH signal and the VHH-LTR colocalising signal were also taken into account. Primary images after 120 minutes of incubation are shown in FIGS. 7A-7F.

After 60 minutes, the percentage intralysosomal VHH1, 5 and 7 reaches equilibrium whereas VHH8 is coming to a plateau at 90 minutes. LTR-positive voxels of cells treated with VHH1, -8 and rhGAA contained up to 60% of the internalized protein while the total VHH7-positive LTR-positive pool was around 20% after three hours. The triangled curves outline the monitored fraction of LTR-stained organelles that colocalize with an AF488-VHH or -rhGAA. The total LTR-pool, positive for AF488 signal was the highest for VHH7, being between 30-40% after three hours, and was around 15% for VHH1. The fraction of the LTR-pool containing VHHs was less than 10% for VHH5, VHH8 and rhGAA and even lower for the other VHHs. Overall, these results clearly indicate specific endocytosis and highest percentage of lysosomal targeting with anti-CI-M6PR VHH1, 5, 7 and 8 (when compared to the negative control (i.e. GBP); Table 2). The positive control shows only limited lysosomal colocalization.

TABLE 2
Percentage of intralysosomal VHHs and percentage
of lysosomes colocalizing with VHH after
60 minutes incubation on MCF7 cells.

| | % cellular VHH | % of lysosomes |
|---|---|---|
| VHH | in lysosomes | containing VHH |
| anti-GFP VHH | 2.082 | 0.016 |
| anti-CI-M6PR VHH1 | 59.045 | 4.086 |
| anti-CI-M6PR VHH2 | 19.744 | 0.150 |
| anti-CI-M6PR VHH3 | 99.452 | 0.249 |
| anti-CI-M6PR VHH4 | 78.595 | 0.3797 |
| anti-CI-M6PR VHH5 | 89.436 | 1.551 |
| anti-CI-M6PR VHH6 | 4.590 | 0.035 |
| anti-CI-M6PR VHH7 | 25.908 | 9.473 |
| anti-CI-M6PR VHH8 | 62.290 | 6.642 |
| anti-CI-M6PR VHH9 | 38.810 | 0.259 |
| anti-CI-M6PR VHH10 | 74.60317461 | 0.123 |
| anti-CI-M6PR VHH11 | 0.279 | 0.002 |

Although labelled with a differential efficiency, the variation in endolysosomal content for these four anti-CI-M6PR VHHs upon endocytosis may indicate a differential lysosomal delivery or a variable cycling path for these molecules. This is the most pronounced for VHH7, for which we observed—compared to the others—a lower VHH7-endolysosomal pool but an increased fraction of endolysosomes containing VHH7. To explore whether these variations in endolysosomal content were the result of true lysosomal delivery, we investigated the fraction of AF488-VHH colocalizing with LAMP1, a lysosomal membrane protein increasingly present in mature lysosomes, on fixed cells. We did this for VHH7—which is increasingly endocytosed—and also for VHH8, for which a low intracellular fraction but larger LTR-positive fractions could be observed. After their incubation for four hours on Hela cells, an anti-LAMP1 antibody was used for staining.

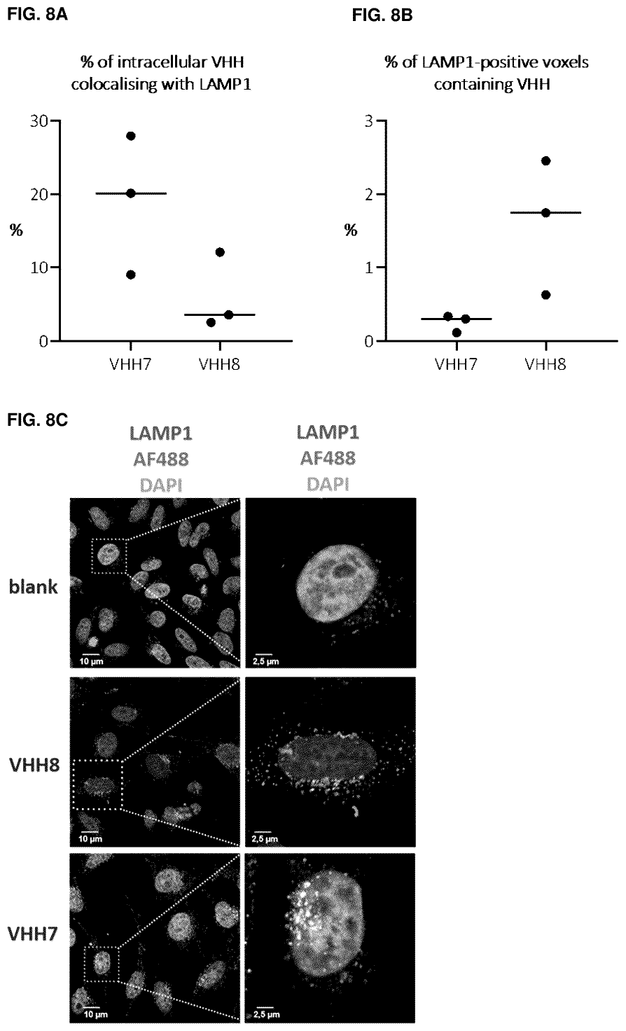

As shown in FIGS. 8A-8C, the intralysosomal fraction of intracellular VHH7 after 240 minutes in the same range (i.e. 19%) as to what we observed during live-cell imaging experiments after 200 minutes with LTR (i.e. 20%). However, this is entirely opposite for VHH8 as we detected only 2.5-12% intracellular VHH8 colocalising with LAMP1-positive lysosomes compared to the 60% with LTR. Next to the intralysosomal fraction, we also calculated the percentage of LAMP1-stained voxels containing VHH (FIG. 8A).

It is remarkable that the LAMP1-colocalising and LTR-colocalising fraction of AF488-VHH7, being 19% and 20% respectively, is similar while VHH8 has a much higher LTR-colocalising fraction (i.e. 60%) compared to the LAMP1-colocalising fraction (i.e. maximally 12%). Although it is difficult to compare live-cell imaging and microscopic examination after fixation, also of course because of the different cell lines used, these experiments possibly suggest that VHH7 and VHH8, may follow different endolysosomal paths. VHH7 shows a more or less equal colocalisation with LTR and LAMP1, suggesting VHH7's increased lysosomal targeting. Given VHH7's five times higher dissociation rate at pH 6.0 versus pH 7.4, it is plausible that immediate endosomal acidification upon endocytosis allows VHH7 to quickly dissociate after which it can be delivered to the lysosome together with the endosomal cargo during maturation.

In that case, the unbound receptor is recycled and may participate in a new round of binding. The low amount of VHH8 in LAMP1-stained compared to LTR-stained organelles could indicate an increased colocalisation with the earlier endolysosomal network. However, a higher amount of LAMP1-lysosomes with VHH8 was detected (FIG. 8B). Because VHH8's transition in dissociation between pH 6.0 and pH 5.0, it is plausible that it may remain bound to hCI-M6PR at the early endosomal stage (pH 5.9-6.5) instead of being delivered to the lysosome. The high LAMP1-colocalisation of VHH7 on the one hand and the peripheral localisation of VHH8, on the other hand, can be indicative of this (FIG. 8C).

It should also be noted that once the VHHs reach the mature lysosome, they would probably be denatured by lysosomal proteases. What then happens to the fluorophore in terms of localisation is unknown. However, we can assume that this behaviour will be similar across the studied VHHs.

Important throughout the interpretation of the absolute counts in these microscopic examinations is to be aware of the obtained degree of labelling of every VHH and its reduced affinity for the recombinant hCI-M6PR_{D1-D3}. While this is unavoidable and comparable to what is generally expected for these NHS-ester labels, absolute counts of VHH7 are probably overestimated due to its high labelling efficiency. It is also important to recall that the divergent affinity combined for recombinant hCI-M6PR_{D1-D3} does not directly correspond binding to the native hCI-M6PR. Moreover, as we calculated fractions of colocalising AF488 and LTR or LAMP1 signal, it was observed, with all other noted, that low-level endocytosis with variability among the replicate VHHs can quickly result in aberrantly high colocalising percentages. The other VHHs had no evidence of CI-M6PR receptor engagement at the cell surface in previous experiments; yet, most did show this low-level highly variable uptake, similar to GBP and were excluded for these reasons from the above discussion.

### Example 5. Humanized Variants of VHH7 and VHH8

Multiple humanized variants of the anti-CI-M6PR VHHs VHH7 and VHH8 were designed in silico (SEQ ID NOs: 26-29 for VHH7 humanized variants and SEQ ID NOs: 30-35 for VHH8 humanized variants; as disclosed in Callewaert et al. (PCT/EP2022/054278)). VHH7hWN and VHH8hWN were produced in HEK293S and purified through IMAC and SEC. The variants VHH7h1-3 and VHH8h1-5 were produced in E. coli and purification was performed through IMAC and desalting.

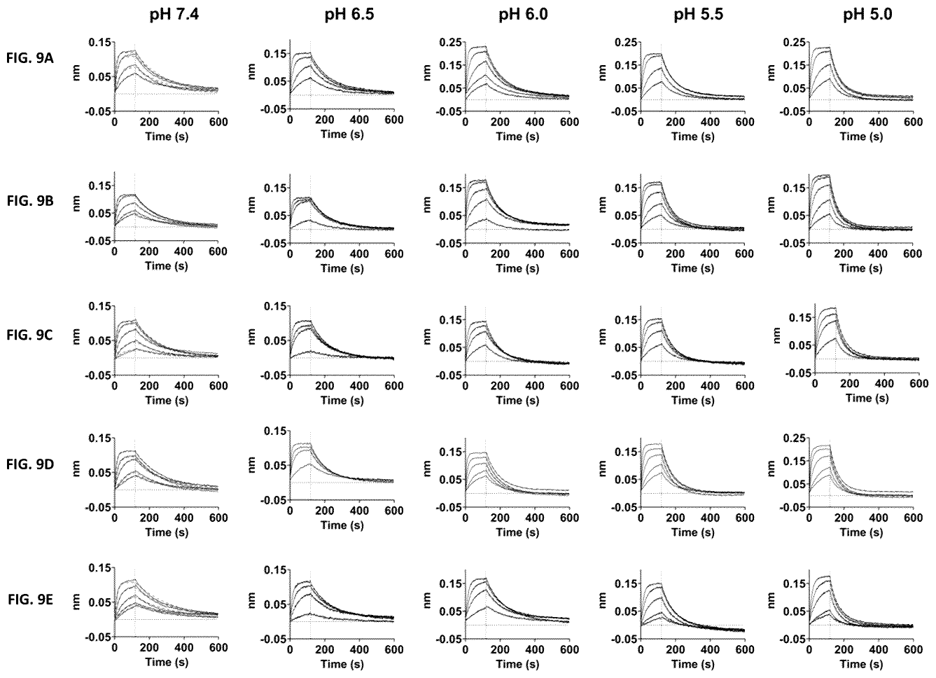

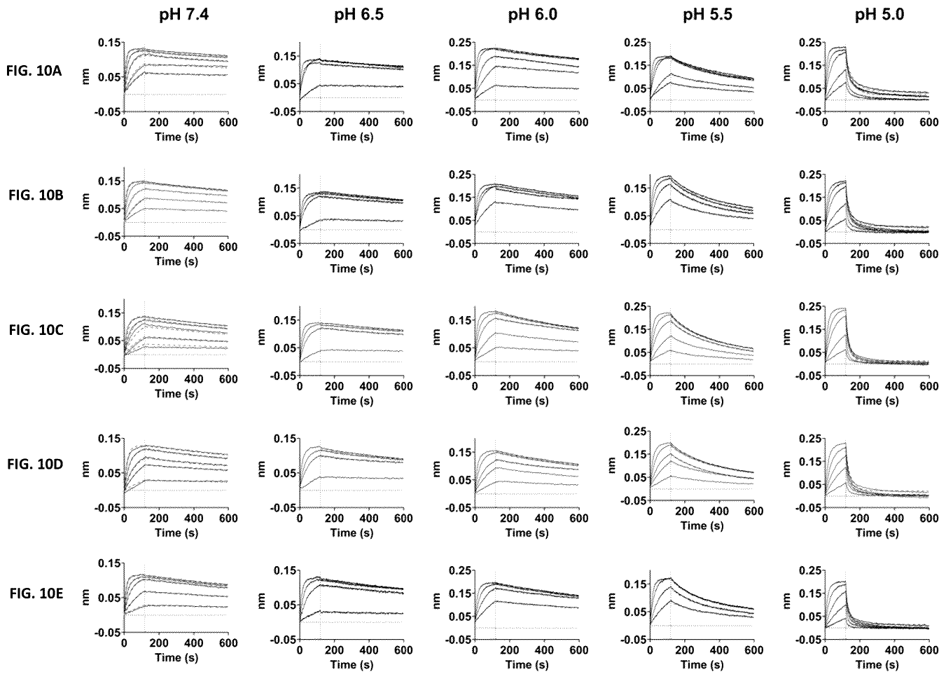

For a selection of the humanized variants of VHH7 and VHH8, a biolayer interferometry (BLI) experiment was performed in which the human CI-M6PR domain₁₋₃His6 was biotinylated and coupled to streptavidin biosensor tips. After loading, the tips were incubated with VHHs serially diluted in pH 7.4 kinetic buffer during the association phase and dissociation was performed at pH 7.4, pH 6.5, pH 6.0, pH 5.5 and pH 5.0. All biosensor tips were then regenerated before analysis of the subsequent VHH. Table 3 summarizes the kinetic parameters retrieved after processing and curve fitting of the BLI measurements. When both association and dissociation were performed at pH 7.4, a global fit was performed according to the 1:1 binding model of which the resulting affinity constants (K_{D}), association (kₒₙ) and dissociation rate constants (k_{off}) are shown. For measurements with association at pH 7.4 and dissociation at pH 6.5, 6.0, 5.5 and 5.0, the depicted dissociation rate constants are an average of the parameters determined by local curve fitting of the dissociation of 200, 100 and 50 nM VHH. Association-dissociation curves for VHH7 and its humanized variants are shown in FIGS. 9A-9E and those for VHH8 and its humanized variants are depicted in FIGS. 10A-10E.

BLI revealed pH-dependent dissociation of the humanized variants VHH7h1, VHH7h2, VHH7h3 and VHH7hWN, with dissociation profiles similar to their non-humanized counterpart VHH7, with the dissociation rate gradually but moderately increasing with decreasing pH (FIGS. 9A-9E, Table 3). Furthermore, their affinity for the human CI-M6PR domain₁₋₃His₆ at pH 7.4 remains almost unaltered upon humanization. Likewise, the pH-dependent dissociation profile of VHH8, where the dissociation rate only moderately increases between pH 7.4 and 6.0, but then demonstrates a rapid surge with close to one order of magnitude between pH 5.5 and 5.0, is unaltered for its humanized variants VHH8h1, VHH8h2, VHH8h3 and VHH8hWN (FIGS. 10A-10E, Table 3). Here also, the obtained K_{D}-value at pH 7.4 is comparable for all variants under evaluation.

TABLE 3
Overview of kinetics parameters determined through BLI of the binding of VHH7, VHH8
and a selection of their humanized variants to human CI-M6PR domain₁₋₃His₆.

| \|VHH | pH | K_{D} (M) | k^{On} (M⁻¹s⁻¹) | k_{Off} (s⁻¹) | VHH | pH | K_{D} (M) | k^{On} (M⁻¹s⁻¹) | k_{Off} (s⁻¹) |
|---|---|---|---|---|---|---|---|---|---|
| VHH7 | 7.4 | 1.01 × 10⁻⁸ | 5.4 × 10⁵ | 5.5 × 10⁻³ | VHH8 | 7.4 | 5.34 × 10⁻¹⁰ | 6.1 × 10⁵ | 3.2 × 10⁻⁴ |
| | 6.5 | | | 9.1 × 10⁻³ | | 6.5 | | | 8.4 × 10⁻⁴ |
| | 6.0 | | | 9.9 × 10⁻³ | | 6.0 | | | 9.1 × 10⁻⁴ |
| | 5.5 | | | 1.3 × 10⁻² | | 5.5 | | | 3.4 × 10⁻³ |
| | 5.0 | | | 1.7 × 10⁻² | | 5.0 | | | 2.3 × 10⁻² |
| VHH7h1 | 7.4 | 1.41 × 10⁻⁸ | 5.0 × 10⁵ | 7.1 × 10⁻³ | VHH8h1 | 7.4 | 1.06 × 10⁻⁹ | 4.5 × 10⁵ | 4.8 × 10⁻⁴ |
| | 6.5 | | | 9.7 × 10⁻³ | | 6.5 | | | 5.3 × 10⁻⁴ |
| | 6.0 | | | 1.2 × 10⁻² | | 6.0 | | | 1.5 × 10⁻³ |
| | 5.5 | | | 1.4 × 10⁻² | | 5.5 | | | 4.3 × 10⁻³ |
| | 5.0 | | | 2.0 × 10⁻² | | 5.0 | | | 2.9 × 10⁻² |
| VHH7h2 | 7.4 | 1.36 × 10⁻⁸ | 4.7 × 10⁵ | 6.4 × 10⁻³ | VHH8h2 | 7.4 | 3.07 × 10⁻⁹ | 2.2 × 10⁵ | 6.7 × 10⁻⁴ |
| | 6.5 | | | 9.4 × 10⁻³ | | 6.5 | | | 1.1 × 10⁻³ |
| | 6.0 | | | 1.1 × 10⁻² | | 6.0 | | | 1.5 × 10⁻³ |
| | 5.5 | | | 1.4 × 10⁻² | | 5.5 | | | 4.4 × 10⁻³ |
| | 5.0 | | | 1.9 × 10⁻² | | 5.0 | | | 4.1 × 10⁻² |
| VHH7h3 | 7.4 | 1.40 × 10⁻⁸ | 4.6 × 10⁵ | 6.4 × 10⁻³ | VHH8h3 | 7.4 | 1.76 × 10⁻⁹ | 2.9 × 10⁵ | 5.1 × 10⁻⁴ |
| | 6.5 | | | 1.1 × 10⁻² | | 6.5 | | | 2.2 × 10⁻³ |
| | 6.0 | | | 1.3 × 10⁻² | | 6.0 | | | 2.0 × 10⁻³ |
| | 5.5 | | | 1.5 × 10⁻² | | 5.5 | | | 4.1 × 10⁻³ |
| | 5.0 | | | 1.9 × 10⁻² | | 5.0 | | | 3.3 × 10⁻² |
| VHH7hWN | 7.4 | 1.44 × 10⁻⁸ | 3.0 × 10⁵ | 4.4 × 10⁻³ | VHH8hWN | 7.4 | 1.29 × 10⁻⁹ | 4.4 × 10⁵ | 5.7 × 10⁻⁴ |
| | 6.5 | | | 8.6 × 10⁻³ | | 6.5 | | | 1.3 × 10⁻³ |
| | 6.0 | | | 9.5 × 10⁻³ | | 6.0 | | | 1.2 × 10⁻³ |
| | 5.5 | | | 1.1 × 10⁻² | | 5.5 | | | 5.1 × 10⁻³ |
| | 5.0 | | | 1.8 × 10⁻² | | 5.0 | | | 3.7 × 10⁻² |

### Example 6. Multi-Angle Light Scattering and Crystallography of VHH-hDom1-3his Complexes

In view of identifying the binding sites of the VHHs used herein, structural analysis of several VHHs in complex with the human M6PR domains 1-3 was performed, as previously described in Callewaert et al. (PCT/EP2022/054278). Because the three N-terminal domains of CI-M6PR are repeats of CI-M6PR type domains (Pfam PF00878), we first determined the molecular mass and oligomeric state of the hCIMPR_{D1-D3}:VHH protein complex, we analysed SEC-MALLS-eluted and fractionated samples after incubating hCIMPR_{D1-D3}: VHH8 in a 1:1 and 1:3 molar fashion. The calculated protein masses corresponded to what was expected for the VHH and antigen, 17 kDa (±1 kDa) and 51.3 kDa (±0.9 kDa) respectively, and 62 kDa (±2 kDa) for the complex, which complies to an equimolar binding of both proteins. Aggregated or other oligomeric structures could be detected but remain limited, also when fractionated samples were analyzed on non-reducing SDS-PAGE. The complexation of VHH and antigen proteins was also independent from hCI-M6PR_{D1-D3} N-glycans, as investigated after endoglycosidase H digest. Because we found an equimolar binding of VHH8 and hCI-M6PR_{D1-D3}, also equal concentrations of VHH7 and hCIM6PR_{D1-D3} were used during a next SEC-MALLS run. Molecular masses of 50 kDa (±2 kDa) for hCIM6PRD_{D1-D3}, 15 (±1 kDa) for anti-CI-M6PR VHH7 and 63.7 (±0.5 kDa) for the complex were measured. A final preparative SEC run was therefore performed accordingly for both VHH7, VHH8 and VHH1H11 (the latter being obtained after re-panning experiments, see Example 7) with (glycosylated) hCI-M6PR_{D1-D3} before the co-crystallisation screening. After crystallisation, the presence of both the antigen and the VHH was verified on SDS-PAGE.

The N-terminal first three domains of the CI-M6PR (CI-M6PR_{D1-D3}), resemble previously published conformations. In the co-crystal with VHH7 and VHH8, hCI-M6PR_{D1-D3} adopts a trefoil-shaped structure similar to a conformation observed for bovine CI-MPR_{D1-D3} (pdb 1q25).

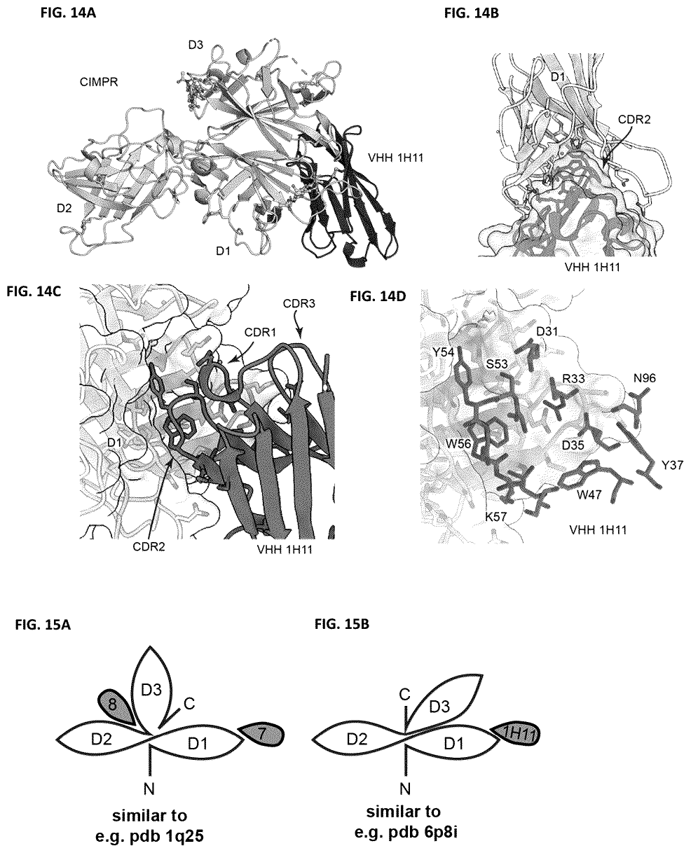

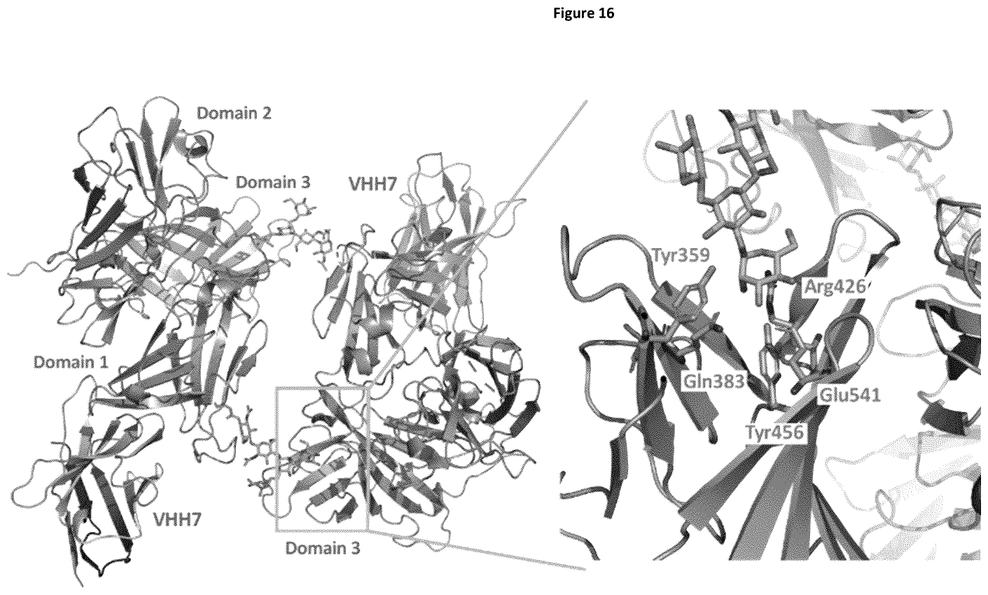

In the co-crystal structure with VHH1H11 the third domain has shifted towards D1 to resemble the conformation present in pdb 6p8i (FIGS. 14A-14D). While present in the crystallisation mixture of VHH7 and -8-containing complexes mannose-6 phosphate was not observed in any of the structures. N-glycans at the three N-glycosylation sequons (i.e. Asn112, Asn400 and Asn435) could be identified to varying degrees from the electron density. In the co-crystal structures with VHH7 and VHH8 clear electron density could be interpreted for a Man3GlcNAc2 or Man4GlcNAc2 containing glycan at Asn112. Only partial core GlcNAc or GlcNAc2 could be interpreted from the electron density at the other positions. Interesting however are the crystal contacts, observed in these crystal structures between the N-linked glycan on Asn112 and the M6P binding pocket in D3 of the crystallographic symmetry-related CI-M6PR copy. More specifically the α1,3-Man of the oligomannosylated glycan on Asn112 binds a cleft on CI-M6PR_{D3} interacting with residues Tyr359, Gln383, Arg426, Glu451 and Tyr456 mostly via hydrogen bonds (FIG. 16). The N-linked glycans on Asn112 and Asn435 of the VHH 1H11 co-crystal structure could be identified as core 1-6 fucosylated.

The core structure of each domain consists of a flattened B-barrel (Pfam domain CIMR PF00878) comprising a five-stranded antiparallel B-sheet (B3-B6) with its strand running orthogonally oriented over a second five-stranded B-sheet (B8-B11), of which the fourth strand interjects between B9 and B11. Each domain should contain four disulfide bonds, as comparable to the bovine crystal structure of the N-terminal three domains of the CI-M6PR (PDB: 1sy0, 1sz0, 1q25,6p8i)¹⁷. The N-terminus of human domain 2 (residues 161-313) and domain 3 (residues 314-467) each contain a linker region composed of a random coil followed by two ancillary β-strands (β0, β1 and β2) that connect the core-flattened β-barrel structures.

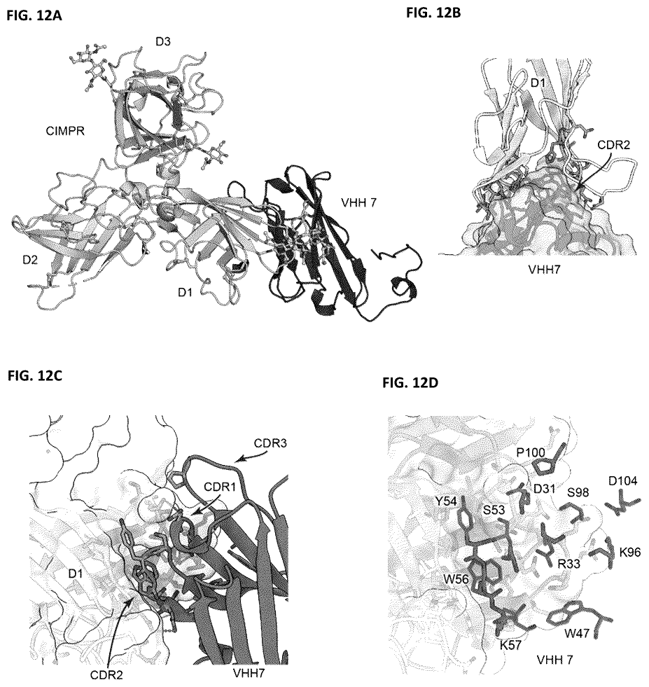

Anti-CI-M6PR VHH7, VHH8 and VHH 1H11 adopt the general immunoglobulin-like fold with a neutral, and stretched-twist turned CDR3 loop respectively. The highest resolution crystal structure of the anti-CI-M6PR VHH7 and hCI-M6PR_{D1-D3} protein complex was solved to a resolution of 2.2 Å (FIG. 12A) and was grown at pH 6.5 (FIG. 12A). The first protein complex reveals a unilateral positioned VHH7 that is packed in between the two B-sheets of hCI-M6PR_{D1}'s flattened B-barrel (FIG. 12B). While presenting one flank to its antigen, VHH7 interacts via its CDR1, 2 but also with residues in CDR3 (FIG. 12C). These make contacts with the amino acid side chains of the intradomain loops A-D of D1 (FIG. 12). This complex is nearly identical in the other crystal form.

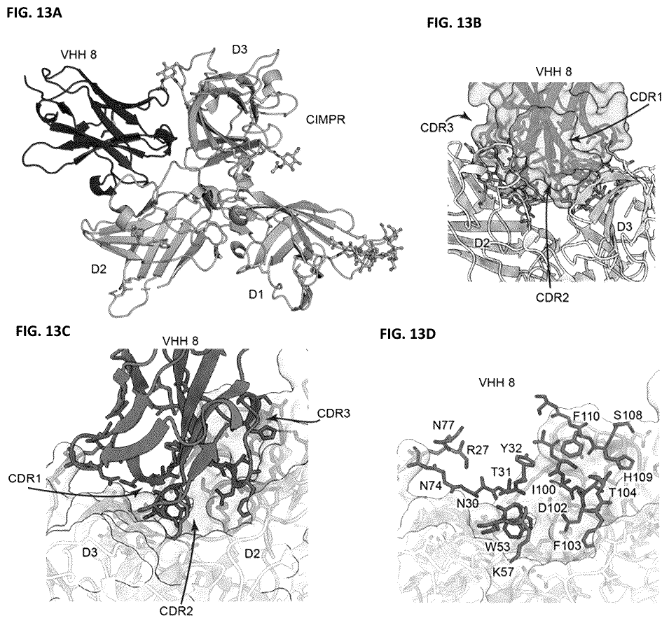

The VHH8 co-crystal structure which was solved to a resolution of 2.75 Å reveals VHH8 is situated in between hCI-M6PR_{D2} and hCI-M6PR_{D3} of the CI-M6PR (FIG. 13A). These form a V-shaped surface from which the amino acids contact the variable protruding loops of VHH8 (FIG. 13B). In general, most of the residues from CDR2 interact with residues of D3, whereas the residues from CDR3 are faced towards D2. The contribution of CDR1 is, compared to the other CDRs, only limited for the overall interaction (FIG. 13C).

The crystal structure of the VHH7-competing anti-CI-M6PR VHH 1H11 and hCI-M6PR_{D1-D3} was solved to a resolution of 2.7 Å and thereby confirmed the results obtained from the mutational screening and competitive BLI. Comparable to VHH7, VHH 1H11 faces hCI-M6PR_{D1}'s flattened β-barrel unilaterally (FIG. 14A-B) and interacts with residues from both β-sheets with CDR1 and CDR2 predominantly (FIG. 14C). As a general overview, a schematic representation of the binding of the lead anti-CI-M6PR VHHs is shown in FIGS. 15A and 15B.

The PISA and FastContact software were consulted to roughly calculate and identify the interacting residues at the binding surface of anti-CI-M6PR VHH7, -8 and -1H11 with hCI-M6PR_{D1-D3}. Because FastContact analysis is biased towards electrostatic interactions, we combined the calculations of the desolvation free energy and electrostatic energy with distance measures, calculated in PyMol to approximate the interfacing residues of anti-CI-M6PR VHHs and its antigen. From this information, two very different para- and epitopes could be delineated for either VHH7, VHH 1H11 and VHH8. The epitope of anti-CI-M6PR VHH7 (FIGS. 12A-12D) mainly consists of amino acids that are part of the intradomain loops A, B, C and D of the β-sheets in CI-M6PRD1 (FIGS. 12A-12D). In addition, hydrophobic residues (e.g. Phe143) that make up the hydrophobic core of the flattened βbarrel contribute to the VHH binding. According to current estimations, important paratope residues comprise Arg33, Lys57 and Asp104 and interact with hCI-M6PR_{D1} residues Asp 87, Glu148 and Lys89 respectively (Table 4, FIG. 12D). These calculations allowed us to confirm the similarity of the epitopes of VHH 1H11 and VHH7. Generally, residues estimated to contribute to the interaction were comparable to the epi- and paratope of VHH7 (Table 4). The residues Arg33 and Lys57 for example, were estimated to be highly involved in the binding of VHH 1H11 to CI-M6PR_{D1-D3} (FIG. 14D), whereas residues of the VHH 1H11's CDR3 are probably contributing less. On top of this, the estimations here showed a high similarity between both epitopes with Asp 87, Lys 89 and Glu148 as highly contributing residues (Table 5). The epitope of anti-CI-M6PR VHH8 is highly different (FIGS. 13A-13D). In contrast to VHH7, interactions of CI-M6PR_{D1-D3} with VHH8 occur with inter- and intradomain loops of hCI-M6PR_{D2} and hCI-M6PR_{D3} but also residues within the β-strands of these domains are impactful (Table 6). As described, amino acids that constitute CDR2 contact D3. Of these, the Lys57 of VHH8 is estimated to form an electrostatic interaction with Glu409 and Glu433 of D3 (Table 6, FIG. 13D). Strong contacts between D2 and CDR3 were estimated to be Asp102 and Lys191 respectively (Table 6, FIG. 13D).

The epitope information allows us to further discuss the (non-) cross-reactive binding of VHH7, -8 and 1H11. Despite a sequence identity of 75% between the human Domain 1-3 and either Bos taurus or Mus musculus domain 1-3 sequences, the VHH7 and VHH8 interface is rather conserved. In FIG. 11, we indicated each of the specific epitope residues in the orthologous sequences for hCI-M6PR_{D1-D3}. A higher degree of variation can be observed for VHH7 than for VHH8 when taking into account residues that contribute significantly to the total binding free energy (i.e. AG below-1.5 kcal/mol).

For Tables 4-6 corresponding to the information on VHH 7, VHH 1H11, and VHH8, resp., the interacting residues and their estimated type of interactions as the estimated binding free energy (ΔG) determined by the sum of the calculated electrostatic free energy, desolvation free energy and configuration entropy for the interaction between the residues of anti-CI-M6PR VHH and hCI-M6PR_{D1-D3} by FastContact and PISA are shown.

TABLE 4
Overview of the epi- and paratopes of anti-
CI-M6PR VHH7 binding the rhCI-M6PR_{D1-D3}.

| | | | Estimated | Estimated |
|---|---|---|---|---|
| | VHH7 | hCI-MPR_{D1-D3} | type of | binding free |
| | residues | residues | interaction | energy (kcal/mol) |
| CDR1 | Asp 31 | Lys 59 | Electrostatic | ΔG < −1.0 |
| | Arg 33 | Asp 57 | Polar | ΔG < 0.0 |
| | Arg 33 | Asp 87 | Electrostatic | ΔG < −4.0 |
| | Arg 33 | Asn 60 | Polar | ΔG < −1.0 |
| | Asp 35 | Lys 89 | Electrostatic | ΔG < −1.0 |
| CDR2 | Ser 53 | Asn 60 | Polar | ΔG < −1.0 |
| | Tyr 54 | Asp 57 | Polar | ΔG < 0.0 |
| | Tyr 54 | Ala 146 | Polar | ΔG < 0.0 |
| | Tyr 54 | Thr 147 | Polar | ΔG < 0.0 |
| | Tyr Sa | Phe 143 | Hydrophobic | ΔG < 0.0 |
| | Trp 56 | Met 85 | Polar | ΔG < 0.0 |
| | Trp 56 | Gla 148 | Polar | ΔG < 0.0 |
| | Lys 57 | Glu 148 | Electrostatic | ΔG < −4.0 |
| | Lys 57 | Asp 118 | Electrostatic | ΔG < −10 |
| CDR3 | Lys 96 | Asp 87 | Electrostatic | ΔG < −0.0 |
| | Asp 104 | Lys 89 | Electrostatic | ΔG < −4.0 |

TABLE 5
Overview of the epi- and paratopes of anti-
CI-M6PR VHH 1H11 binding the rhCI-M6PRD1-D3.

| | | | Estimated | Estimated |
|---|---|---|---|---|
| | VHH 1H11 | bCI-MPR_{D1-D3} | type of | binding free |
| | residues | residues | interaction | energy (kcal/mol) |
| CDR1 | Asp 31 | Lys 59 | Electrostatic | ΔG < −4.0 |
| | Asn 32 | Lys 59 | Electrostatic | ΔG < −1.0 |
| | Arg 33 | Asp 87 | Electrostatic | ΔG < −4.0 |
| | Arg 33 | Asn 60 | Polar | ΔG < −1.0 |
| | Arg 33 | Thr 90 | Polar | ΔG > 0.0 |
| | Arg 33 | Asp 57 | Polar | ΔG < 0.0 |
| | Asp 35 | Lys 89 | Electrostatic | ΔG < −4.0 |
| CDR2 | Thr 50 | Asp 87 | Polar | ΔG < 0.0 |
| | Ala 52 | Asn 60 | NA | ΔG < 0.0 |
| | Ser 53 | Asn 60 | Polar | ΔG < −1.0 |
| | Tyr 54 | Ala 146 | NA | ΔG < 0.0 |
| | Tyr 54 | Glu 148 | Polar | ΔG: NA |
| | Tyr 54 | Arg 404 | Polar | ΔG: NA |
| | Gly 55 | Glu 148 | NA | ΔG > 0.0 |
| | Trp 56 | Ala 146 | NA | ΔG > 0.0 |
| | Trp 56 | Thr 147 | Polar | ΔG > 0.0 |
| | Trp 56 | Glu 148 | Polar | ΔG < 0.0 |
| | Trp 56 | Met 85 | NA | ΔG < 0.0 |
| | Lys 57 | Gln 119 | Polar | ΔG < 0.0 |
| | Lys 57 | Asp 118 | Electrostatic | ΔG < 0.0 |
| | Lys 57 | Glu 148 | Electrostatic | ΔG < −1.0 |
| CDR3 | Asn 96 | Lys 89 | Polar | ΔG < −1.0 |
| | Ser 97 | Lys 89 | Polar | ΔG: NA |
| | Gly 98 | Lys 89 | Polar | ΔG: NA |

TABLE 6
Overview of the epi- and paratopes of anti-
CI-M6PR VHH8 binding the rhCI-M6PRD1-D3.

| | VHHB | hCI-MPR_{D1-D3} | E nated type | Estimated binding free |
|---|---|---|---|---|
| | residues | residues | interaction | energy (kcal/mol) |
| CDR1 | Tyr 32 | Arg 219 | Polar | ΔG < −0.0 |
| CDR2 | Arg 52 | Asp 409 | Polar | ΔG < −1.0 |
| | Trp 53 | Phe 457 | Hydrophobic | ΔG < −0.0 |
| | Ser 54 | Asn 431 | Polar | ΔG < −0.0 |
| | Ser 55 | Asp 409 | Polar | ΔG < −0.0 |
| | Ser 56 | Glu 433 | Polar | ΔG < −0.0 |
| | Lys 57 | Gly 408 | Electrostatic | ΔG < −1.0 |
| | Lys 57 | Asp 409 | Electrostatic | ΔG < −4.0 |
| | Lys 57 | Glu 433 | Electrostatic | ΔG < −4.0 |
| FR2 | Arg 72 | Glu 433 | Polar | ΔG < −0.0 |
| | Asp 73 | Lys 357 | Polar | ΔG < −1.0 |
| | Asn 74 | Lys 357 | Polar | ΔG < −1.0 |
| CDR3 | Ile 100 | Gly 194 | Hydrophobic | ΔG < −0.0 |
| | Ile 100 | Ala 195 | Hydrophobic | ΔG < −0.0 |
| | Val 101 | Ala 195 | Hydrophobic | ΔG < −0.0 |
| | Val 101 | Phe 208 | Hydrophobic | ΔG < −0.0 |
| | Val 101 | Leu 225 | Hydrophobic | ΔG < −0.0 |
| | Asp 102 | Lys 391 | Electrostatic | ΔG < −4.0 |
| | Asp 102 | Ala 195 | Polar | ΔG < −0.0 |
| | Asp 102 | Leu 197 | Polar | ΔG < −0.0 |
| | Phe 108 | Tyr 196 | Hydrophobic | ΔG < −1.0 |
| | Phe 108 | Leu 197 | Hydrophobic | ΔG < −0.0 |
| | Phe 108 | Ile 297 | Hydrophobic | ΔG < −1.0 |
| | Ser 108 | Gln 224 | Polar | ΔG < −0.0 |

Verification of the novelty and uniqueness of the human M6PR binding sites defined herein for VHH7, VHH1H11 and VHH8 required to screen the panel of anti-CI-M6PR VHHs that were developed against the extracellular part of CI-M6PR by LinXis BV (as described in Houthoff et al. published as WO2020/185069A1). In-tandem competitive biolayer interferometry of the purified alternative anti-CI-M6PR VHHs revealed that LinXis VHHs 13E8, as well as the VHH7 and VHH8, described herein, each specifically bound to non-overlapping epitopes on CI-M6PR hDom₁₋₃His₆, and no binding to the CI-M6PR hDom₁₋₃His₆ was observed for any further representative VHH, and thus no competing binders were identified.

### Example 7. Additional VHH CDR3-Families Competing for Binding to the VHH7 or VHH8 Human M6PR Epitope

The original VHH-library of the llama that yielded an antigen-specific response to immunization with recombinant human CI-M6PR Dom1-3His₆ was re-panned onto coated CI-M6PR hDom₁₋₃His₆, as previously described in Callewaert et al. (PCT/EP2022/054278). 15 new VHHs belonging to 12 novel CDR3-groups were identified in these panning efforts.

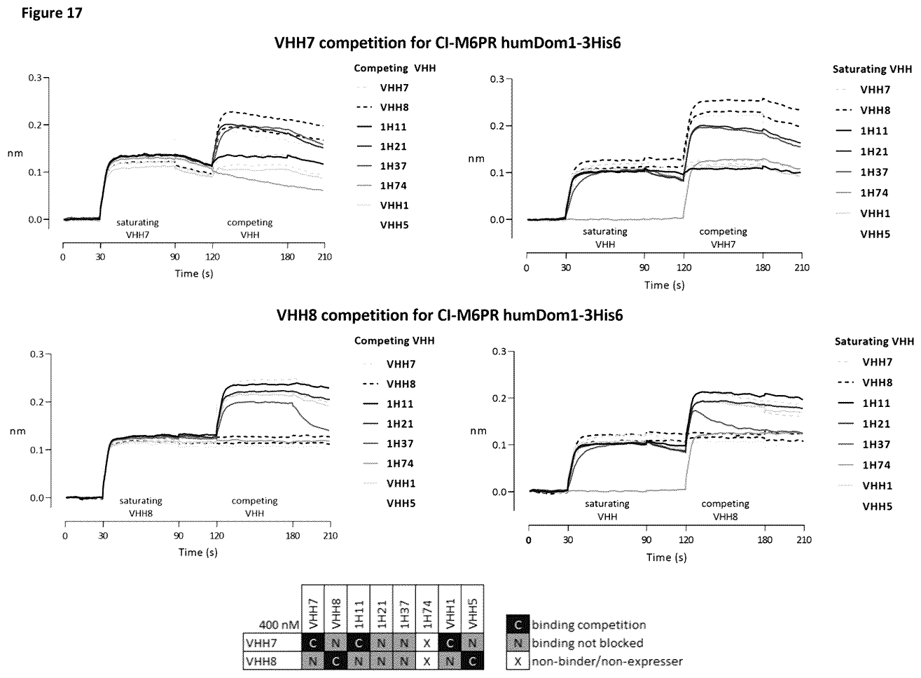

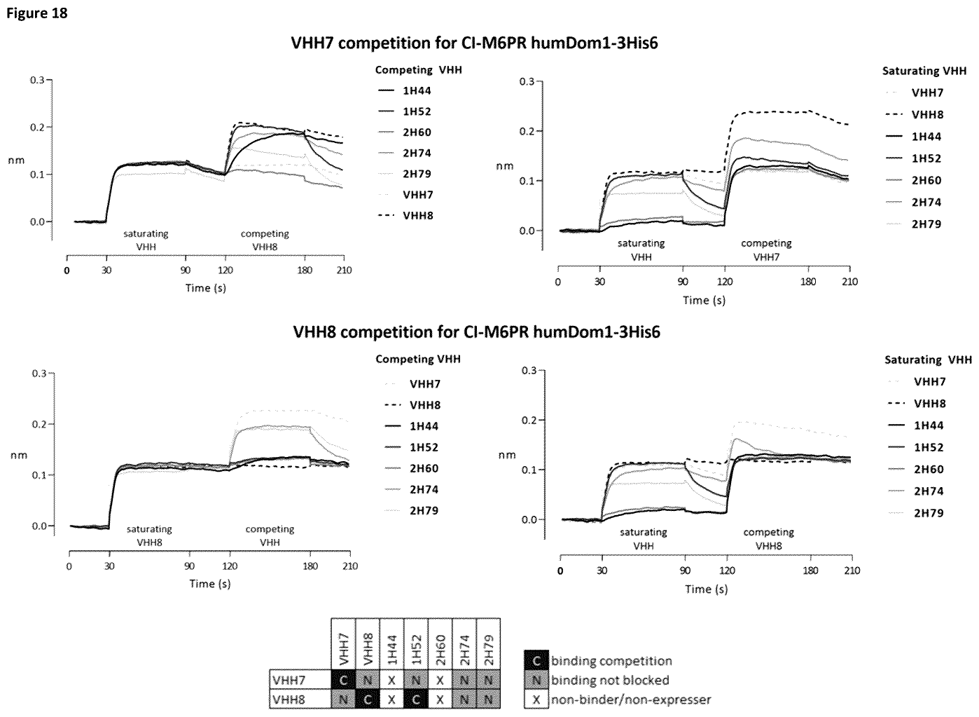

In order to identify anti-CI-M6PR VHHs that bind an overlapping or identical epitope on CI-M6PR as VHH7 or VHH8 from a different ‘CDR3’-family, or VHH family, as defined herein, biolayer interferometry (BLI) experiments were performed for identification of competitors of VHH7 or VHH8 for the CI-M6PR binding site, wherein previously characterized VHH1 and VHH5 were also evaluated. In-tandem competitive BLI of anti-CI-M6PR VHHs purified from E. coli revealed that VHHs 1H11 and VHH1 competed with VHH7 for CI-M6PR hDom₁₋₃His₆ binding but not with VHH8; and 1H52 and VHH5 competed with VHH8 for CI-M6PR hDom₁₋₃His₆ binding but not with VHH7, whereas 1H21, 1H37, 2H74 and 2H79 did not compete for binding to CI-M6PR hDom₁₋₃His₆ with VHH7 or VHH8. No saturating binding of CI-M6PR hDom₁₋₃His₆ was obtained for 1H74, 1H44 and 2H60 (FIG. 17, 18).

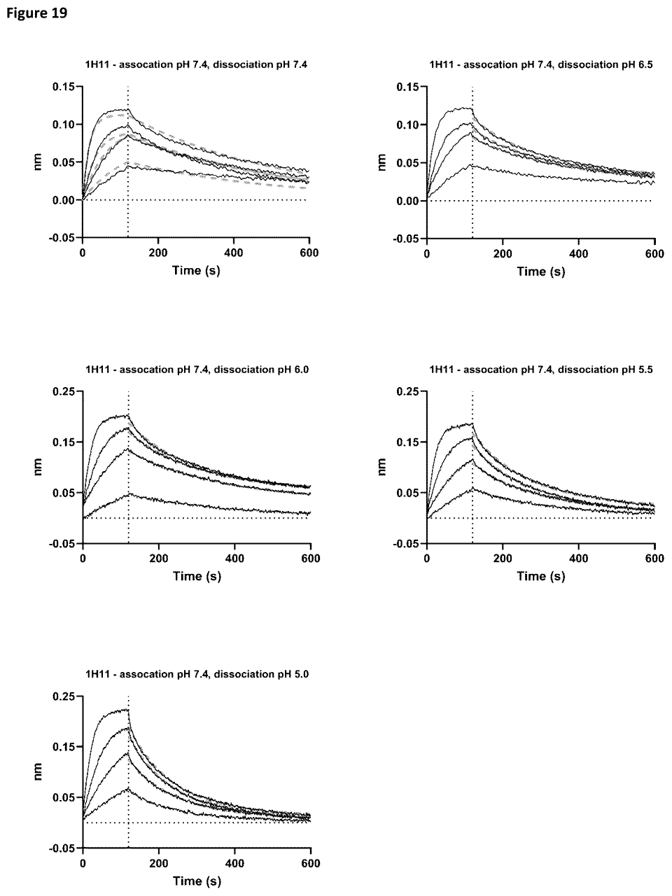

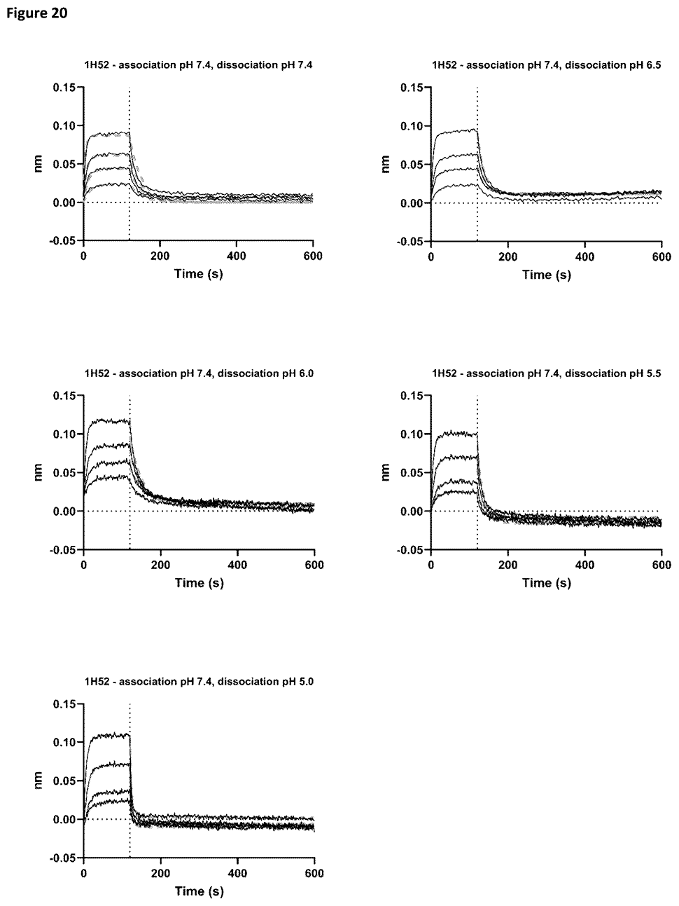

For VHH 1H11 (SEQ ID NO: 24) and VHH 1H52 (SEQ ID NO: 25), a BLI experiment was performed in which the human CI-M6PR domain₁₋₃His₆ was biotinylated and coupled to streptavidin biosensor tips. After loading, the tips were incubated with VHHs serially diluted in pH 7.4 kinetic buffer during the association phase and dissociation was performed at pH 7.4, pH 6.5, pH 6.0, pH 5.5 and pH 5.0. All biosensor tips were then regenerated before analysis of the subsequent VHH. Table 7 summarizes the kinetic parameters retrieved after processing and curve fitting of the BLI measurements. When both association and dissociation were performed at pH 7.4, a global fit was performed according to the 1:1 binding model of which the resulting affinity constants (K_{D}), association (kₒₙ) and dissociation rate constants (k_{off}) are shown. For measurements with association at pH 7.4 and dissociation at pH 6.5, 6.0, 5.5 and 5.0, the depicted dissociation rate constants are an average of the parameters determined by local curve fitting of the dissociation of 200, 100 and 50 nM VHH. Association-dissociation curves for VHH1H11 and VHH1H52 are shown in FIG. 19 and FIG. 20, respectively.

Analysis of the BLI data revealed that anti-CI-M6PR VHH 1H11, for which competition for binding of CI-M6PR hDom₁₋₃His₆ with VHH7 was demonstrated (in addition to VHH1) through BLI, demonstrates a similar pH-dependent dissociation profile as VHH7 itself (FIG. 19). As is the case for VHH7, values for the dissociation rate constant gradually but moderately increase with decreasing pH from pH 7.4 down to pH 5.0 (Table 7). VHH 1H52, one of the anti-CI-M6PR VHHs that competed with VHH8 for binding of CI-M6PR hDom₁₋₃His₆ (next to VHH5) as shown through BLI, also showed a similar pH-dependent dissociation profile as VHH8 (FIG. 20). Indeed, there is a rapid increase in the rate of dissociation between pH 5.5 and pH 5.0 (Table 7).

TABLE 7
Overview of binding data analysis as determined
by BLI for the binding of VHH 1H11 and VHH
1H52 to human CI-M6PR domain1-3His6.

| VHH | pH | K_{D} (M) | k_{On} (M⁻¹s⁻¹) | k_{Off} (s⁻¹) |
|---|---|---|---|---|
| 1H11 | 7.4 | 4.90 × 10⁻⁹ | 5.1 × 10⁵ | 2.5 × 10⁻³ |
| | 6.5 | | | 4.8 × 10⁻³ |
| | 6 | | | 5.1 × 10⁻³ |
| | 5.5 | | | 6.3 × 10⁻³ |
| | 5 | | | 8.3 × 10⁻³ |
| 1H52 | 7.4 | 3.10 × 10⁻⁸ | 1.0 × 10⁶ | 3.2 × 10⁻² |
| | 6.5 | | | 6.7 × 10⁻² |
| | 6 | | | 3.0 × 10⁻² |
| | 5.5 | | | 5.1 × 10⁻² |
| | 5 | | | 2.0 × 10⁻¹ |

### Example 8. Production and Purification of VHH-Based Anti-EGFR NanoLYTAC-Constructs

LYTAC-constructs directed against EGFR were cloned, containing the anti-EGFR VHH 9G8 S54A, coupled at the C-terminus with a (G4S)₃-linker (or additionally a (G4S)₉-linker) to either the anti-CI-M6PR VHH VHH7 or VHH8. Furthermore, LYTAC-constructs containing two copies of 9G8 S54A, also coupled with a (G4S)₃-linker, linked to VHH7 or VHH8 were cloned. As controls for internalization and/or degradation not specifically mediated by the CI-M6PR, we designed constructs linking either one or two copies of 9G8 S54A to the anti-GFP VHH named ‘GBP’. All of these constructs were produced in Pichia pastoris with a C-terminal FLAG3His₆ tag and purified through IMAC and desalting of which the expression yields are summarized in Table 8, as well as the composition of the protein constructs. The quality of the protein constructs was checked through SDS-PAGE (FIGS. 22a and 22B) and their HeLa-cell binding was verified through flow cytometry (data not shown).

TABLE 8
Composition and Pichia pastoris expression yield of VHH-
based anti-EGFR nanoLYTAC constructs and controls.

| | | | | Anti-CI- | |
|---|---|---|---|---|---|
| Construct | VHH fusion | | | M6PR | Total yield (mg) |
| Nr. | SEQ ID NO: | Anti-EGFR moiety | Linker | moiety | (100 ml culture) |
| 34 | 82 | 9G8 S54A | G4Sx3 | VHH7 | 11.4 |
| 35 | 18 | 9G8 S54A | G4Sx3 | VHH8 | 16.0 |
| 36 | 84 | 9G8 S54A − G4Sx3 − | G4Sx3 | VHH7 | 11.4 |
| | | 9G8 S54A | | | |
| 37 | 19 | 9G8 S54A − G4Sx3 − | G4Sx3 | VHH8 | 10.6 |
| | | 9G8 S54A | | | |
| 38 | 20 | 9G8 S54A | G4Sx3 | GBP | 18.2 |
| 39 | 21 | 9G8 S54A − G4Sx3 − | G4Sx3 | GBP | 10.2 |
| | | 9G8 S54A | | | |
| 40 | 88 | 9G8 S54A | G4Sx9 | VHH7 | 9.4 |
| 41 | 89 | 9G8 S54A | G4Sx9 | VHH8 | 9.9 |

‘G4Sx3’ = triple Gly4Ser linker

Of the LYTACs containing VHH8 and the control constructs, an initial set of proteins was produced earlier in small scale (constructs 26-29, as described in examples 1-3) that, however, N-terminally potentially contained 3 additional amino acids related to the cloning method comprising the amino acids Arg-Ser-Met (RSM) prior to the sequences as provided herein. As described in this example, those constructs were re-cloned with the absence of those additional amino acids and re-produced, after which their retained in vitro EGFR-internalization efficacy was validated through flow cytometry (Example 9). The proteins without the N-terminal additions have thus been used throughout the experiments and examples further provided, with results aligning to those initially observed.

### Example 9. Evaluation of In Vitro EGFR-Internalization Efficacy of VHH-Based Anti-EGFR NanoLYTAC-Constructs Through Flow Cytometry

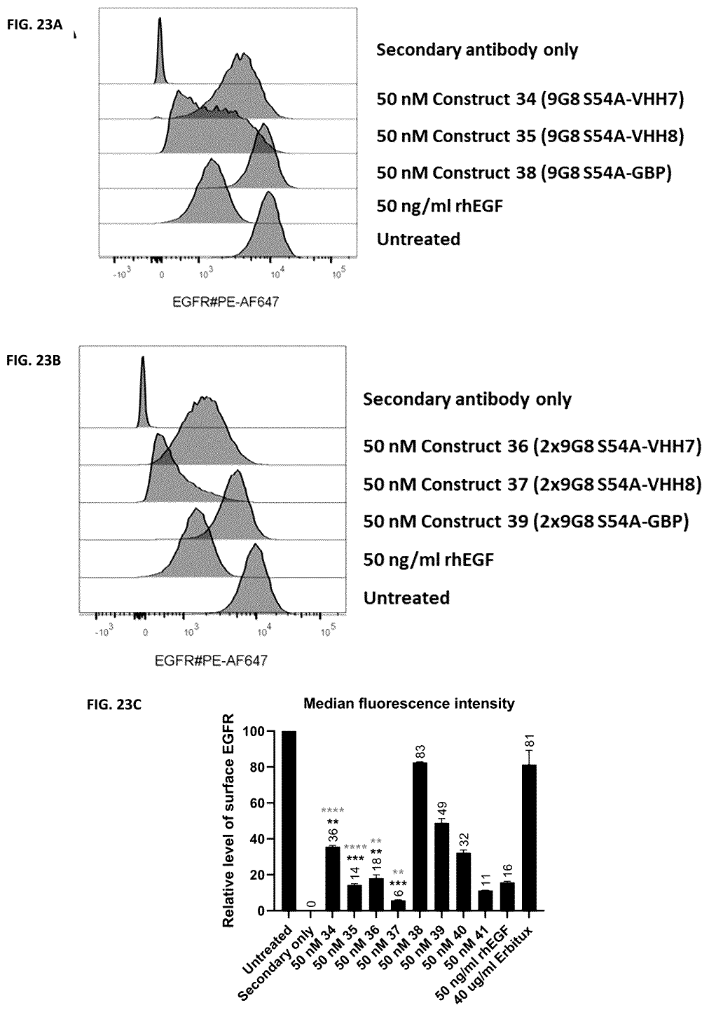

The efficacy of the VHH-based anti-EGFR nanoLYTAC-constructs to lower the levels of EGFR at the cell surface was evaluated in vitro. To this end, Hela cells were incubated in complete medium with 50 nM of the LYTAC-constructs (number 34-37, see Table 8) or the control constructs during 24 h, followed by detachment of the cells, staining for cell-surface EGFR and measurement through flow cytometry. In FIG. 23A, representative histograms of the fluorescent signal corresponding to cell-surface EGFR were set out for untreated Hela cells and Hela cells treated with 50 ng/ml recombinant human EGF (rhEGF), LYTAC-constructs 34 (9G8 S54A-VHH7), 35 (9G8 S54A-VHH8) or the corresponding control construct 38 (9G8 S54A-GBP). FIG. 23B, depicts representative histograms for untreated Hela cells and Hela cells treated with 50 ng/ml rhEGF, LYTAC-constructs 36 (2x 9G8 S54A-VHH7), 37 (2x9G8 S54A-VHH8) or the corresponding control construct 39 (2x9G8 S54A-GBP). In FIG. 23C, a bar chart is shown indicating the median fluorescence intensity (MFI) values measured for all conditions in the experiment, normalized to the MFI measured for untreated Hela cells and expressed in percentages.

The results of this experiment demonstrate that the signal for cell-surface EGFR was significantly reduced for cells treated with either of the LYTAC-constructs that are monovalent for EGFR (constructs 34 & 35) in comparison to the untreated cells (34: P=0.001, 35: P=0.0007) and to the cells treated with the corresponding control construct (38) (34: P=0.00009), 35: P=0.00005) that also induces a modest EGFR-internalization effect (to 83% of the untreated cells). The monovalent EGFR-binding constructs with a longer (G4S)₉-linker (constructs 40 & 41) also demonstrated efficient induction of EGFR internalisation, comparable to (and in this experiment even slightly more efficient than) their (G4S)₃-counterparts.

Furthermore, a lower signal was detected for Hela cells treated with either of the LYTAC-constructs bivalent for EGFR (constructs 36 & 37) as compared to the untreated cells (constructs 36: P=0.001, 37: P=0.0006) and to the cells treated with the corresponding control construct (39) (constructs 36: P=0.005, 37: P=0.002). Moreover, the bivalent EGFR-binding LYTACs (constructs 36 & 37) induced an even more efficacious internalization of EGFR in Hela cells than the monovalent EGFR-binding LYTACs (constructs 34 & 35). Note that the signal corresponding to cell-surface EGFR for cells treated with the bivalent control construct (construct 39) is also lowered to 49% of the signal for untreated Hela cells, an expected effect as a result of bivalent binding of EGFR.

### Example 10. Evaluation of In Vitro EGFR-Degradation Efficacy of VHH-Based Anti-EGFR NanoLYTAC-Constructs

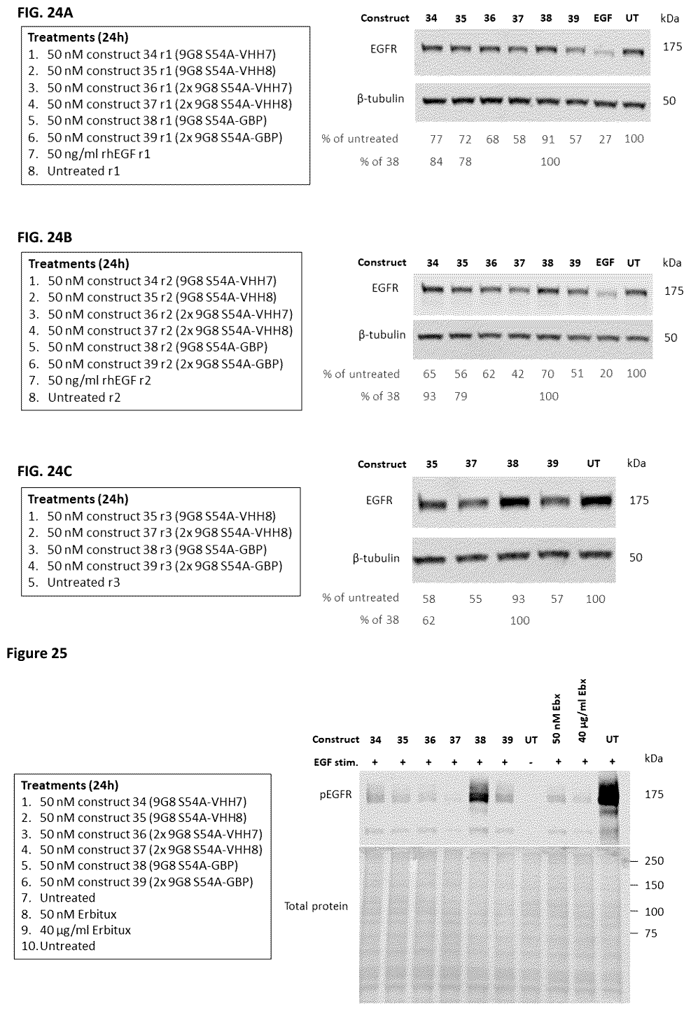

As to evaluate the efficacy of the VHH-based anti-EGFR nanoLYTAC constructs to induce degradation of the target protein, a Western Blot assay was optimized. To this end, Hela cells were incubated during 24 h in complete growth medium—as opposed to serum-free optiMEM as detailed in Example 3—with 50 nM of the anti-EGFR LYTACs (constructs 34-37) or the corresponding control constructs (38-39) or with 50 ng/ml rhEGF as a positive control for EGFR degradation. Cell lysates were then obtained in RIPA buffer and equal amounts of protein (as determined through a BCA-assay) were subjected to immunoblotting for fluorescent detection—as opposed to chemiluminescent detection in Example 3—of EGFR and beta-tubulin (FIGS. 24A-24C).

From the quantified intensity values corresponding to total EGFR, determined through densitometry, it is demonstrated that there is a consistently lower signal for the cells treated with either of the LYTAC-constructs (34-37) as compared to the untreated cells. Furthermore, when normalized to the signal for lysates treated with the monovalent control construct 38 (9G8 S54A-GBP), it is clear that there is a consistently lower value for the cells treated with either of the monovalent LYTAC-constructs (34 & 35) indicating a CI-M6PR-dependent effect, confirming that these completely VHH-based anti-EGFR nanoLYTAC-constructs can effectively induce degradation of EGFR. We strongly suspect that our earlier inability to detect lower levels of total EGFR as induced by the VHH8-containing anti-EGFR nanoLYTAC-constructs (26 & 27) (as described in Example 3) was due to both unoptimized cellular assay-related and technical conditions.

### Example 11. Evaluation of In Vitro Inhibition of Ligand-Induced EGFR Activation by VHH-Based Anti-EGFR NanoLYTAC-Constructs

In order to assess whether treatment with the completely VHH-based anti-EGFR nanoLYTAC constructs is effective in inhibiting the ligand-induced activation of EGFR, a Western Blot assay was performed. HeLa cells were treated during 24 h in complete growth medium with 50 nM of the nanoLYTAC-constructs (34-37) or the corresponding controls (constructs 38-39) or with the FDA/EMA-approved therapeutic anti-EGFR monoclonal antibody Erbitux® (50 nM or 40 μg/ml). Following the incubation period, cells were stimulated with 50 ng/ml of recombinant human EGF (rhEGF) during 5 minutes after which cell lysates were obtained in RIPA buffer and equal amounts of protein were subjected to immunoblotting for the fluorescent detection of phosphorylated EGFR (@ Tyr1068) (FIG. 25).

The results demonstrate a significant reduction in the levels of phosphorylated EGFR upon treatment with either of the monovalent EGFR-binding LYTACs (construct 34 & 35) and the bivalent EGFR-binding LYTACs (construct 36 & 37) as compared to the stimulated untreated cells and compared to the cells treated with their corresponding control constructs (38 and 39, respectively). Interestingly, the strength of this effect is in the same range as for treatment with Erbitux, with construct 37 inducing an even stronger inhibitory effect than Erbitux at either of the tested concentrations.

### Example 12. Production and In Vitro EGFR-Internalization and Degradation Efficacy of Cetuximab-VHH Fusions as Anti-EGFR NanoLYTAC-Constructs

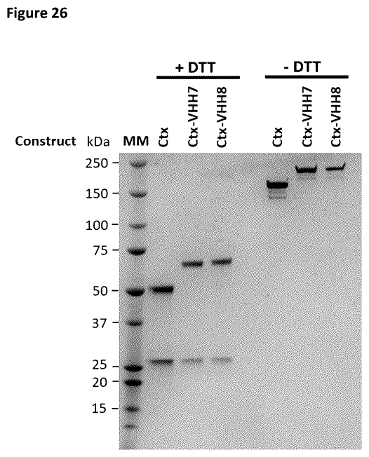

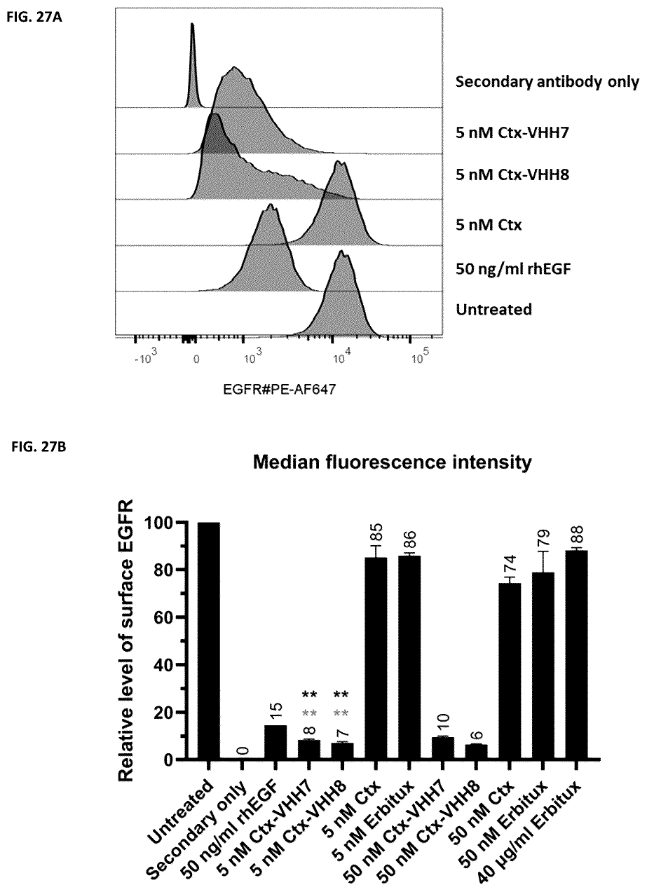

LYTAC-constructs directed against EGFR were designed, consisting of the therapeutic anti-EGFR monoclonal antibody (mAb) cetuximab, coupled at the C-terminus of the Fc-domain with a (G4S)₂-linker to either the anti-CI-M6PR VHH7 or VHH8. These cetuximab-based nanoLYTAC constructs were expressed in Chinese hamster ovary (CHO) cells and purified from the supernatant through protein A chromatography and SEC. As a control for non-CI-M6PR-mediated EGFR-internalization, non-VHH-fused cetuximab was also produced in CHO. The expression yields hereof are indicated in Table 9, as well as a summary of the composition of the protein constructs. Their quality was assessed through SDS-PAGE (FIG. 26).

TABLE 9
Composition and CHO cell expression yield of cetuximab-
based anti-EGFR nanoLYTAC constructs and controls.

| | | | | | Anti-CI- | Total yield |
|---|---|---|---|---|---|---|
| Construct | Heavy chain | Light chain | Anti-EGFR | | M6PR | (mg) (100 ml |
| name | SEQ ID NO: | SEQ ID NO: | moiety | Linker | moiety | culture) |
| Ctx | 87 | 86 | cetuximab | / | / | 1.17 |
| Ctx-VHH7 | 88 | 86 | cetuximab | G4Sx2 | VHH7 | 1.84 |
| Ctx-VHH8 | 89 | 86 | cetuximab | G4Sx2 | VHH8 | 1.76 |

‘G4Sx2’ = double Gly4Ser linker

The efficacy of these cetuximab-VHH fusion constructs to lower the levels of EGFR at the cell surface was evaluated in vitro. To this end, Hela cells were incubated with either 5 or 50 nM of the LYTAC-constructs (Ctx-VHH7 or Ctx-VHH8) or the control constructs during 24 h, followed by detachment of the cells, staining for cell-surface EGFR and measurement through flow cytometry.

In FIG. 27A, representative histograms of the fluorescent signal corresponding to cell-surface EGFR were set out for untreated Hela cells and Hela cells treated with 50 ng/ml recombinant human EGF (rhEGF), 5 nM of the LYTAC-constructs (Ctx-VHH7 or Ctx-VHH8) or the corresponding control construct (Ctx). In FIG. 27B, a bar chart is shown indicating the median fluorescence intensity (MFI) values measured for all conditions in the experiment, normalized to the MFI measured for untreated Hela cells and expressed in percentages.

The results of this experiment demonstrate that the signal for cell-surface EGFR was significantly reduced for cells treated with 5 nM of either of the cetuximab-based nanoLYTAC-constructs (Ctx-VHH7 & Ctx-VHH8) in comparison to the untreated cells (Ctx-VHH7: P=0.002, Ctx-VHH8: P=0.002) and to the cells treated with the corresponding control construct (Ctx) (Ctx-VHH7: P=0.002, Ctx-VHH8: P=0.002) that also induces an expected modest EGFR-internalization effect. The induced effect size is not further increased at the higher (50 nM) treatment concentration.

Interestingly, the size of the EGFR-internalization effect in HeLa-cells induced by the said cetuximab-VHH fusion constructs seems to be larger than the one induced by the mannose-6-phosphonate (M6Pn)-functionalized LYTAC-constructs described by Banik et al. and Ahn et al. [15], and in Bertozzi et al. WO2020132100A1. Indeed, when expressed relative to the cell-surface EGFR level of untreated HeLa cells, treatment with 50 nM Ctx-VHH7 reduced the level to 10% and treatment with 50 nM Ctx-VHH8 reduced the level to 6%, while 50 nM of the M6Pn-Ctx construct only reduces the EGFR-level to 25-30% (as read from the bar chart shown in FIG. 4C Ahn et al. [15]). We believe that the effect size measured in these flow cytometry experiments can be reliably compared due to the following reasons: (1) both experiments are conducted on Hela cells, (2) in both experiments the same primary EGFR-detection antibody is used (EGFR monoclonal antibody 199.12, #MA5-13319, Invitrogen), (3) the said LYTAC-constructs utilize the same EGFR-binding moiety being the monoclonal antibody cetuximab and (4) treatments were conducted with the same concentration of each LYTAC-construct. Taking into account that no side by side comparison was made, the differences are still significant enough to conclude that this indicates that the CI-M6PR binding VHHs as disclosed in Callewaert et al. (PCT/EP2022/054278) and used for incorporation in the LYTAC-constructs described herein are more effective in inducing internalization of a target protein in the context of a bispecific construct than the M6Pn-ligand.

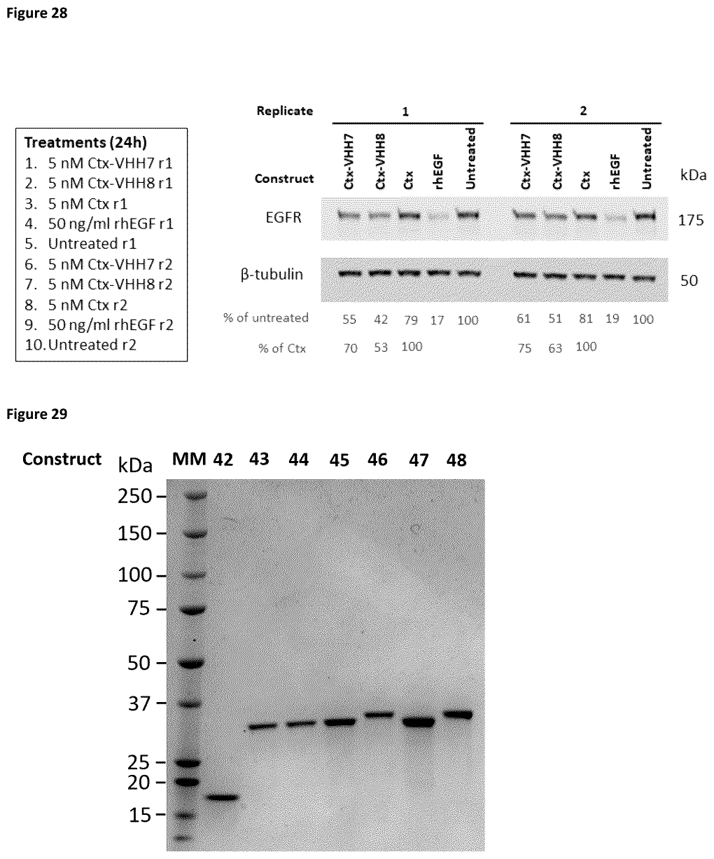

As to evaluate the efficacy of the cetuximab-VHH fusions as nanoLYTAC constructs to induce degradation of EGFR, a Western Blot assay was performed. Hela cells were incubated during 24h in complete growth medium with 5 nM of Ctx-VHH7 or Ctx-VHH8 or of the corresponding non-VHH-fused control Ctx or with 50 ng/ml of rhEGF as a positive control for EGFR degradation. After the treatment period, cell lysates were obtained in RIPA buffer and equal amounts of protein (as determined through a BCA-assay) were subjected to immunoblotting for fluorescent detection of EGFR and beta-tubulin (FIG. 28).

From the quantified intensity values corresponding to total EGFR, determined through densitometry, it is demonstrated that there is a consistently lower signal for the cells treated with each of the LYTAC-constructs as compared to the untreated cells. Furthermore, when normalized to the signal for lysates treated with the non-VHH fused control construct (Ctx), it is clear that there is a consistently lower value for the cells treated with either of the LYTAC-constructs (Ctx-VHH7 & Ctx-VHH8) indicating a CI-M6PR-dependent effect, confirming that the described cetuximab-VHH fusion constructs can effectively induce degradation of EGFR.

### Example 13. Production and In Vitro GFP-Internalization and Degradation Efficacy of VHH-Based Anti-GFP LYTAC Constructs

As to gain proof of concept that a CI-M6PR VHH-based LYTAC construct can also effectively induce internalization and degradation of a soluble antigen, LYTAC-constructs directed against GFP were produced, containing the anti-GFP VHH ‘GBP’ coupled at the C-terminus with a (G4S)₃-linker to either the anti-CI-M6PR VHH ‘VHH7’ or ‘VHH8’. Additionally, anti-GFP LYTAC-constructs coupled to the ‘VHH7’-competing VHHs ‘VHH1’ and ‘VHH 1H11’ and coupled to the ‘VHH8’-competing VHHs ‘VHH5’ and ‘VHH 1H52’ were produced. As a control for non-CI-M6PR-mediated internalization and degradation of GFP, monovalent GBP was also included in this set. These constructs, of which the composition is summarized in Table 10, were produced in Pichia pastoris with a C-terminal FLAG3His₆ tag and purified through IMAC and desalting. Their quality was assessed through SDS-PAGE (FIG. 29).

TABLE 10
Composition of the VHH-based anti-GFP nanoLYTAC constructs
and controls produced in Pichia pastoris.

| Construct | VHH fusion | Anti-GFP | | Anti-CI-M6PR |
|---|---|---|---|---|
| Nr. | SEQ ID NO: | moiety | Linker | moiety |
| 42 | 90 | GBP | / | / |
| 43 | 91 | GBP | G4Sx3 | VHH7 |
| 44 | 92 | GBP | G4Sx3 | VHH8 |
| 45 | 93 | GBP | G4Sx3 | VHH1 |
| 46 | 94 | GBP | G4Sx3 | VHH5 |
| 47 | 95 | GBP | G4Sx3 | VHH 1H11 |
| 48 | 96 | GBP | G4Sx9 | VHH 1H52 |

‘G4Sx3’ = triple Gly4Ser linker

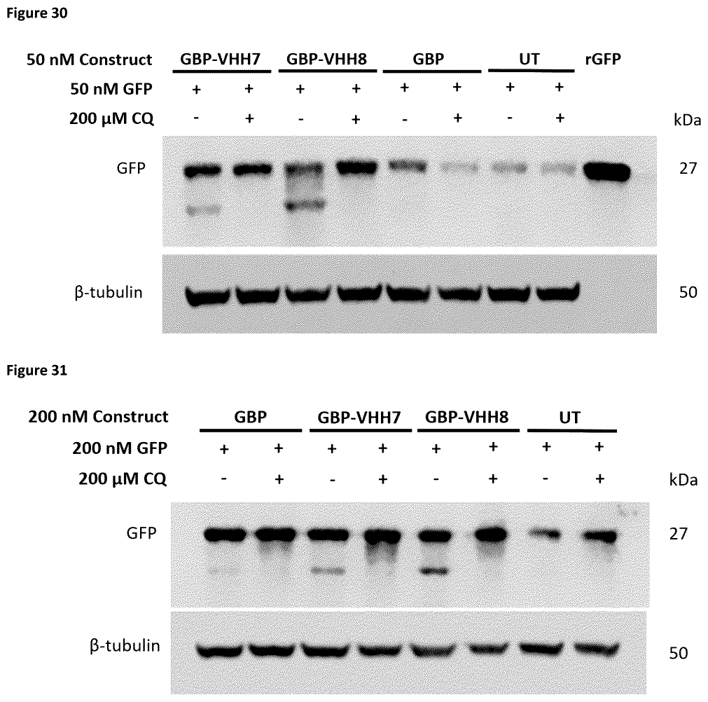

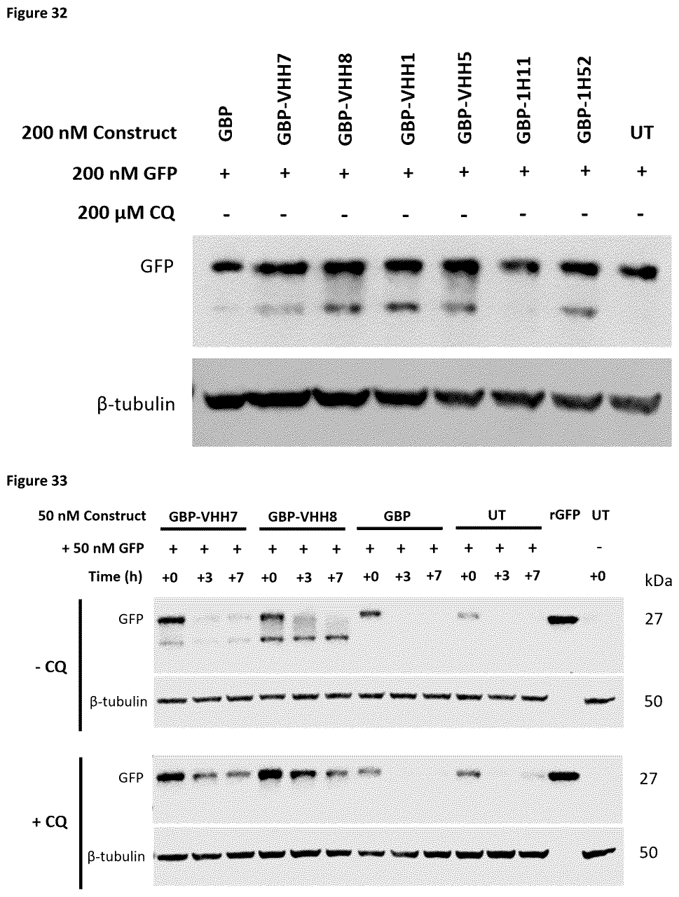

In first instance, we assessed whether the VHH7- and VHH8-containing anti-GFP LYTACs could induce cellular internalization and degradation of GFP in vitro. To this end, 50 nM of these LYTAC-constructs (43=GBP-VHH7 & 44=GBP-VHH8) or of the corresponding non-CI-M6PR-VHH-fused control (42=GBP) was pre-incubated during 30 minutes at room temperature with 50 nM of recombinant GFP (rGFP) in serum-free OptiMEM (Gibco). Hela cells were then incubated with these protein solutions or with a solution containing only GFP during 24h either with or without the presence of 200 μM chloroquine, a well-described lysosomotropic compound that inhibits endosomal acidification and thus lysosomal degradation. Following the incubation period, cell lysates were obtained and equal amounts of protein (as determined through a BCA-assay) were subjected to immunoblotting for the fluorescent detection of GFP and beta-tubulin as a loading control (FIG. 30). The results indicate that GFP was indeed taken up in the cells treated with the LYTAC-constructs, albeit also to some extent in the cells treated with monovalent GBP and the cells only incubated with GFP. More importantly however, is the appearance of an additional band at a lower molecular weight than expected for full-length GFP and this only in the LYTAC-treated non-chloroquine-treated conditions. This suggests that GFP was degraded upon internalization only in the presence of a GFP-specific LYTAC-construct and this in a CI-M6PR- and lysosome-dependent manner. These results were also validated in MCF7 cells (FIG. 31). Through a similar experiment, the GFP-LYTAC constructs containing the previously described competitor anti-CI-M6PR VHHs of VHH7, being VHH1 and VHH 1H11, and of VHH8, being VHH5 and VHH 1H52, were evaluated in vitro. In this assay however, Hela cells were incubated in complete growth medium with 200 nM of each construct and rGFP and chloroquine-treated samples could not be obtained due to the bad condition of the cells (FIG. 32). The lower-molecular weight band, as had been previously established to be a degradation product of GFP, was here effectively detected for the anti-GFP LYTACs containing VHH7, VHH8, VHH1, VHH5 and VHH 1H52, indicating that bispecific constructs with these anti-CI-M6PR VHHs can also effectively and selectively induce degradation of a target antigen. For the cells treated with the GBP-VHH 1H11 fusion construct, the degradation product of GFP could unfortunately not be detected in this particular assay.

In a follow-up experiment, we wanted to evaluate the cellular fate of internalized GFP after treatment washout. Therefore, Hela cells were again treated with pre-incubated solutions of 50 nM of the LYTACs (constructs 43=GBP-VHH7 & 44=GBP-VHH8) or of monovalent GBP (=42) and 50 nM rGFP with or without chloroquine during 24 h, after which the cells were washed with PBS and incubated with fresh complete growth medium during an additional amount of time. Cell lysates were obtained immediately after the treatment period (±0 h) and at 3 h (±3 h) and 7 h (±7 h) of additional incubation. In the same way as before, the lysates were immunoblotted for detection of GFP and beta-tubulin (FIG. 33). The Western Blot analysis indicated the occurrence of a steady-state process in which GFP was exocytosed to the non-GFP containing medium in the investigated time period after treatment washout for all conditions tested, implied by the gradual disappearance of the signal corresponding to GFP. Besides that, it is implied that the pool of recombinant GFP that was internalized after the initial treatment period is further degraded in a CI-M6PR-dependent manner in the time frame following washout in the non-chloroquine LYTAC-treated conditions as evidenced by the continued detection of the lower-molecular weight band.

### Material and Methods

### Cell Culture

HEK293 suspension cells were cultivated in FREX medium composed of EX-CELL (Gibco 14571C) and Freestyle 293 medium (Gibco) supplemented with L-glutamine (Lonza, 2 mM). Expi-Chinese hamster ovary (CHO) cells were cultivated in ExpiCHO™ Expression Medium (Gibco). Hela cells were cultured in DMEM (0.1 mM non-essential amino acids (NEAA), 2 mM L-glutamine, 1 mM sodium pyruvate, 10% fetal calf serum (FCS)) and incubated with 5% CO2 at 37° C. MCF7 cells were cultivated in DMEM: F12 medium supplemented with FCS (10%) and L-glutamine (2 mM).

Cloning, Production and Purification of VHH-Based Anti-EGFR and Anti-GFP nanoLYTAC Constructs

A modular cloning platform was employed for the generation of expression vectors for Pichia pastoris. Codon-optimized coding sequences were cloned in between the AOX1-promoter and -terminator and a FLAG3His₆-tag was attached C-terminally. The vectors were transformed to competent P. pastoris cells (NCYC2543) through electroporation and the proteins were produced via methanol-induction [11]. The clarified supernatant was supplemented with MgCl2 (25 mM), reduced L-Gluthation (100 mg/L, Sigma Aldrich, G4251-1G). After filtration (0.22 μm) the supernatant was loaded onto a HisTrap HP (5 ml) column (GE Healthcare, 17524801) after which the bound proteins were washed (5 CV of 20 mM imidazole, 0.5 M NaCl, 20 mM NaH2PO4/Na2HPO4, pH 7.5) and gradually eluted (10 CV, 400 mM imidazole, 20 mM NaCl, 20 mM NaH2PO4/Na2HPO4, pH 7.5). Analysis of selected peak fractions was performed on SDS-PAGE (4-20%, Genscript). Fractions containing the protein of interest were pooled and ran on a HiLoad16/10 desalting column, equilibrated with HBS buffer (50 mM HEPES, 150 mM NaCl, pH 7.5).

Cloning, Production and Purification of Cetuximab-Based Anti-EGFR nanoLYTAC Constructs

The human codon optimized coding sequences for Ctx-VHH7, Ctx-VHH8 and Ctx containing the IgG CH signal peptide were ordered synthetically, incubated for 45 minutes at 37° C. with Klenow fragment (3′ to 5′ exo-) (NEB, M0212L), NEBuffer 2 (NEB), dATP (0.1 mM) and cloned using the pcDNA™3.3-TOPO™ TA Cloning™ Kit (Thermo Fischer Scientific, K830001) according to the provided protocol. The cloned plasmids were heat shock transformed (42° C., 90 seconds) into chemically competent E. coli and sequence verified. For recombinant protein production, the corresponding expression vectors were transfected into Expi-Chinese hamster ovary (CHO) suspension cells using the ExpiFectamine™ CHO Transfection Kit (Thermo Scientific). The medium was harvested for purification on day 10 after transfection and the supernatant was loaded on a HiTrap MabSelect SuRe (5 mL) column (Cytiva) after which the bound proteins were washed with Mcilvaine buffer pH 7.2 (0.2 M Na₂HPO₄, 0.1 M citric acid) and eluted with McIlvaine buffer pH 3.0. Analysis of selected peak fractions was performed on SDS-PAGE (4-20%, Genscript). Fractions containing the protein of interest were pooled and ran on a HiLoad 16/600 Superdex 200 pg (Cytiva) and again the eluted fractions were analysed on SDS-PAGE. Positive fractions were pooled and concentrated in HBS-buffer (50 mM HEPES, 150 mM NaCl, pH 7.2).

### Flow Cytometry

Hela cells were cultured as described before, seeded at 100,000 cells per well in 12 well plates and incubated with various concentrations of the LYTAC-constructs or the control constructs during 24 hours. After the incubation period, the cells were harvested using Cell Dissociation Buffer (Gibco) and transferred to Eppendorf tubes for final transfer to a 96 well V-bottom plate. After harvest, the cells were washed 2 times with PBS and once with PBS+0.5% BSA before incubation with anti-EGFR monoclonal antibody (199.12, ThermoFisher, #MA5-13319, 1:40) during 1 hour at 4° C. The cells were washed 3 times and incubated with the secondary anti-mouse IgG PE-AF647 (ThermoFisher, #A-20990, 1:250) during one hour at 4° C. After three additional wash steps with PBS+0.5% BSA, the cells were resuspended in 100 μl PBS+0.5% BSA and transferred to tubes for measurement on the BD LSR II flow cytometer.

### Protein Degradation Analysis by Western Blot

Hela cells and MCF7 cells were cultured as described before and seeded at 300,000 or 450,000 cells per well respectively in 6-well plates. For the in vitro GFP degradation assay, 50 or 200 nM of anti-GFP LYTACs were pre-incubated with an equimolar concentration of recombinant GFP during 30 minutes at room temperature. Cells were then incubated with these protein solutions with or without the addition of 200 μM chloroquine during 24 h. For the in vitro EGFR-degradation assay, cells were incubated with 50 nM of anti-EGFR LYTACs during 24 h. For both assays, cell lysates were obtained in 100 μl RIPA-buffer by scraping with a pipette-tip, agitated during 1 hour at 4° C. and centrifuged at maximal speed to remove cell debris. The protein concentration was determined through a BCA-assay and an equal amount of protein was mixed with 5× Laemmli-buffer containing DTT and incubated at 98° C. during 10 minutes. The proteins were separated on precast 4-20% gradient SDS-PAGE gel (GenScript, M00657) and transferred to a nitrocellulose membrane through a wet-blot method. The membrane was blocked in 5% skim milk in PBST (or 5% BSA in TBST when phosphorylated EGFR is detected) during one hour and incubated with anti-EGFR antibody (EGFR Rabbit mAb (D38B1), Cell Signaling Technology, #4267S), anti-GFP antibody (GFP Rabbit mAb (D5.1), Cell Signaling Technology, #2956) or anti-phoshpoEGFR antibody (phospho-EGFR (Tyr1068) (D7A5) Rabbit mAb, Cell Signaling Technology, #3777) overnight at 4° C. Three washing steps with PBST (or TBST) of 15 minutes each were performed, before incubation with the secondary antibody.

For chemiluminescent detection, the membrane was incubated with HRP-conjugated secondary antibody (Rabbit IgG from donkey, Cytiva, NA934) during one hour at room temperature. Beta-actin was detected as a loading control with a directly-labeled primary antibody (Anti-β-actin antibody C4, Santa Cruz, sc-47778 HRP). After another wash step, the membrane was developed with TMB-substrate solution (Western Lighting Plus-ECL, Perkin Elmer, NEL103001EA) and imaged with the Amersham Imager 680 (Cytiva).

For fluorescent detection, the membrane was incubated during 1 hour at room temperature with a mouse anti-ß-tubulin antibody (Sigma Aldrich, T4026) and, after three washes, incubated with an anti-rabbit Dylight800-conjugated secondary antibody (Thermo Scientific) and an anti-mouse Dylight680-conjugated secondary antibody (Thermo Scientific). Imaging was conducted with an Odyssey Imaging System (LI-COR Biosciences). Densitometric analysis of the Western Blot was performed using ImageJ.

### Recombinant Production of the Human Domain1-3His6 Antigen

HEK293 suspension cells were cultivated in serum-free EX-CELL (Gibco 14571C-1000ML) and Freestyle 293 medium (Gibco) (1:1) supplemented with L-Glutamine (2 mM) and grown at 37° C. in 8% CO₂ while shaking at 125 rpm. pcDNA™3.3-TOPO-hDom₁₋₃His₆ (675 μg) and SV40 Large T antigen DNA (1%) was used for transfection of HEK293 suspension cells (300 mL) with polyethylene imine (1:2) (PolyScience, linear, 25 kDa). The supernatant was harvested 3 days after transfection (200×g, 5′) and supplemented with MgCl2 (2 mM), reduced L-Gluthation (100 mg/L, Sigma Aldrich, G4251-1G) and 1× cOmplete™ Protease Inhibitor (Roche, 11697498001). After filtering (0.22 μm), the supernatant was loaded onto a HisTrap HP (5 ml) column (GE Healthcare, 17524801). After washing (5 CV of 20 mM imidazole, 0.5 M NaCl, 20 mM NaH2PO4/Na₂HPO₄, pH 7.5), the bound proteins were eluted (10 CV, 400 mM imidazole, 20 mM NaCl, 20 mM NaH2PO4/Na₂HPO₄, pH 7.5) and analysed on SDS-PAGE (4-20%, Genscript). Afterwards, the hDomain₁₋₃His₆ positive fractions were loaded on a HiLoad 16/600 Superdex 200 pg (GE Healthcare, 28989335) and eluted fractions were analysed on SDS-PAGE followed by staining with Coomassie β-Blue R250 and positive fractions were pooled and concentrated in MES buffer (50 mM MES, 150 mM NaCl, pH 6.5). The mDom1-3His6 was expressed and produced similarly to the human variant but only purified over a HisTrap (5 mL) column (GE Healthcare, 17524801). The eluted fractions were pooled, concentrated over a Amicon® Ultra-15 Centrifugal Filter Unit (Merck Millipore, UFC901008) and resuspended in MES buffer.

### Production and Purification of Anti-CI-M6PR VHHs

The plasmid (1000 ng) was linearized using PmeI (1U, NEB) and used to transform electrocompetent Pichia pastoris NRRL-Y-11430 by electroporation. Subsequently, Buffered Glycerol Complex Medium for Yeast (pH 6) was used for inoculation of a single clone transformant and growth for 48 h at 28° C. while shaking at 225 rpm. A buffer switch was performed to Buffered Complex Medium for Yeast (pH 6) and cultures were grown for another 48 h at 28° C. while shaking at 225 rpm. Every 12 h, the growing cultures were spiked with methanol (1%). Finally, the supernatant was harvested by centrifugation (1250 rpm, 15′) and adjusted to pH 7.

VHH1, VHH5, VHH6, VHH 1H11 and VHH 1H52 were expressed in E. coli by transforming competent WK6 E. coli cells with the pHEN6c vector containing the VHH open reading frames, the Lac operon, the PelB secretion signal, the ampicillin selection marker and an origin of replication. Transformed E. coli cells were inoculated in LB medium containing ampicillin (100 μg/mL) and incubated overnight at 37° C., while shaking at 200-250 rpm. Of this preculture 1 ml was added to 330 ml TB containing ampicillin (100 μg/mL), MgCl2 (2 mM) and glucose (0.1%) and incubated at 37° C. while shaking until OD600 was 0.6-0.9. When reached the desired OD600, the expression was induced by addition of IPTG (Immunosource Cat® 102A) (1 mM) and the culture was incubated at 28° C., while shaking for 16-18 h. To extract the proteins the overnight-induced cultures were centrifuged for 8 minutes at 8000 rpm and the cell pellet was resuspended from 1 L culture in 12 ml TES by pipetting up and down, followed by shaking for 1 hour at 4° C. Per 12 ml TES, 18 ml TES (1:4 diluted in MQ) was added and further incubated on ice for an additional hour, while shaking. The whole was centrifuged for 30 min at 8000 rpm at 4° C. and the supernatant was used for further purification.

For all VHHs, the clarified supernatant was supplemented with supplemented with MgCl2 (2 mM), reduced L-Gluthation (100 mg/L, Sigma Aldrich, G4251-1G). After filtration (0.22 μm) the supernatant was loaded onto a HisTrap HP (5 ml) column (GE Healthcare, 17524801) after which the bound proteins were washed (5 CV of 20 mM imidazole, 0.5 M NaCl, 20 mM NaH₂PO₄/Na₂HPO₄, pH 7.5) and gradually eluted (10 CV, 400 mM imidazole, 20 mM NaCl, 20 mM NaH₂PO₄/Na₂HPO₄, pH 7.5). Analysis of selected peak fractions was performed on SDS-PAGE (4-20%, Genscript). Fractions containing the protein of interest were pooled and ran on a HiLoad16/10 desalting column, equilibrated with HBS buffer (50 mM HEPES, 150 mM NaCl, pH 7).

### Cloning Production and Purification of Humanized Variants of VHH7 and VHH8

The human codon optimized coding sequences for VHH7hWN and VHH8hWN containing the IgG CH signal peptide and a His₆-tag were ordered synthetically and incubated for 45 minutes at 37° C. with Klenow fragment (3′ to 5′ exo-) (NEB, M0212L), NEBuffer 2 (NEB), dATP (0.1 mM) and cloned using pcDNA™3.3-TOPO™ TA Cloning™ Kit (Thermo Fischer Scientific, K830001) according to the provided protocol. Codon optimized sequences of the humanized variants VHH7h1-3 and VHH8h1-5 were cloned into the pVDS100 vector using the GenBuilder™ cloning kit (GenScript®; cat. no.: L00701) according to the manufacturer's instructions. The cloned plasmids were heat shock transformed (42° C., 90 seconds) into chemically competent E. coli and sequence verified. For recombinant protein production of VHH7hWN and VHH8hWN, the corresponding expression vectors were transfected into HEK293 suspension cells through PEI-transfection. The medium was harvested for purification on day 4 after transfection. VHH7h1-3 and VHH8h1-5 were expressed in E. coli by transforming competent cells with the pVDS100 vector containing the VHH open reading frames. Transformed E. coli cells were inoculated in selective LB medium and incubated overnight at 37° C., while shaking at 250 rpm. The preculture was diluted 1:50 in selective TB-medium supplemented with glucose and lactose for auto-induction of protein expression. The culture was incubated for 2 h at 37° C. while shaking at 250 rpm, after which the temperature was reduced to 30° C. and the culture was incubated for an additional 26h. To extract the proteins the overnight-induced cultures were centrifuged for 20 minutes at 4000 rpm and the cell pellet was resuspended in D-PBS (1/12.5th of the expression volume) by pipetting up and down, followed by shaking for 1 hour at 4° C. The whole was centrifuged for 20 min at 8500 rpm at 4° C. and the supernatant was used for further purification. All supernatants were filtrated (0.22 μm) before purification. Supernatant for VHH7hWN and VHH8hWN was loaded onto a HisTrap HP (5 ml) column (GE Healthcare, 17524801) after which the bound proteins were washed (5 CV of 20 mM imidazole, 0.5 M NaCl, 20 mM NaH₂PO₄/Na₂HPO₄, pH 7.5) and gradually eluted (10 CV, 400 mM imidazole, 20 mM NaCl, 20 mM NaH₂PO₄/Na₂HPO₄, pH 7.5). The VHH-positive fractions were loaded on a HiLoad 16/600 Superdex 75 pg (GE Healthcare) and eluted fractions were analysed on SDS-PAGE. Positive fractions were pooled and concentrated in HBS-buffer (50 mM HEPES, 150 mM NaCl, pH 7). For VHH7h1-3, VHH8h1-5, the IMAC purification was performed on a Janus BioTx system (Perkin Elmer) according to standard procedures. Fractions containing the protein of interest were pooled and buffer exchanged to PBS prior to protein concentration.

Labelling of Anti-CI-M6PR VHHs with Amine Reactive Alexa Fluor 488

Every anti-CI-M6PR VHH (1 mg) was diluted in HEPES (50 mM), NaCl (150 mM) and NaHCO₃ (100 mM), pH 8.3 and incubated with 1 mg Alexa Fluor 488 (AF488) NHS ester (Jena Biosciences, APC-002-5), resuspended in DMSO, for 1 h at room temperature. Afterwards, free AF488 NHS ester was removed using size exclusion chromatography (HiLoad 16/600 Superdex75 pg, GE Healthcare). Eluted fractions were pooled and degree of labelling (DOL) and functional binding to hDom1-3His6 was assessed.

### Microscopic Analysis of Lysosomal Targeting of the Anti-CI-M6PR VHHs

For live-cell imaging, 2×10⁴ MCF7 cells/well were seeded in OptiMEM medium and incubated the next day with Lyso Tracker Deep Red DND-99 (50 nM, Thermo Fischer, L12492) for 30 minutes at 37° C., 5% CO₂ and washed with OptiMEM after which 7.5 μM AF488 labelled anti-CI-M6PR VHHs (in OptiMEM) were incubated on the cells. For every well (i.e. anti-CI-M6PR VHH), Z-stacks were taken at three different positions every six minutes, for three hours in total. Z-slices (12) were acquired per position at a step size of 1.5 μm and XY pixel size was 275 nm by 275 nm. Excitation and emission wavelengths of the fluorescent compounds used were LTR (λ_{Ex}: 633 nm and λ_{Em}: 665-715 nm), AF488 (λ_{Ex}: 488 nm and λ_{Em}: 520±35 nm), Hoechst/DAPI (λ_{Ex}: 405 nm and λ_{Em}: 420-470 nm).

Hela cells were cultivated as previously described and seeded in 8-well chambers (iBidi, 80841) at 2.5×10⁴ cells/well in Ham F-12 medium (supplemented with penicillin and streptomycin) respectively. AF488-labelled VHHs (5 μM) were incubated for four hours on the cells and washed three times with PBS afterwards. Cells were fixed in prewarmed PFA: first in 2% PFA in PBS for 5 minutes at 37° C., and then with 4% PFA for 10 minutes at room temperature. Washing with PBS was performed three times for 5 minutes before and after cell permeabilisation (0.2% Triton X-100) for 10 minutes at room temperature. Cells were then blocked for 30 minutes with normal goat serum diluted (1/100) in PBT buffer. Primary mouse anti-LAMP1 monoclonal antibody (Abcam, Ab25630, 1/500) was diluted in blocking buffer and incubated overnight at 4° C. After washing, 5 minutes each in PBS, the secondary goat anti mouse antibody, coupled to DyLight594 (1/1,000 in PBT) was incubated for two hours at room temperature. The cells were counterstained with DAPI (1/1,000 in PBS) for 15 minutes at 16° C. after washing three times with PBS. Lastly, washed and stained cells were stored at 4° C. after mounting in polyvinyl alcohol.

Imaging was performed on the LSM880 Airyscan confocal microscope (Zeiss, Jena) used in FAST Airyscan SR-mode with a Plan-Apochromat 63x/1.4 Oil DIC M27 objective. For every VHH, optimal Z-stacks—to capture the entire cell volume—were taken at three different positions per well of three fluorescent compounds: LAMP1 (λ_{Ex}: 633 nm and λ_{Em}: >650 nm), AF488 (λ_{Ex}: 488 nm λ_{Em}: 495-550 nm) and Hoechst/DAPI (λ_{Ex}: 405 nm and λ_{Em}: 420-480 nm).

### Image Processing and Analysis

Images acquired with the Airyscan detector were processed using ZEN software (Zeiss, Jena). The processing included pixel reassignment and default Wiener filtering. The processed images were then imported into Volocity (Quorum Technologies, Ontario) for further analysis. Both images acquired on the LSM880 and the Spinning Disk microscope were analyzed with Volocity software. In both cases, thresholds were determined for intensity values and size of segmented objects in the channel of (endo) lysosomal staining and in the channel of labelled VHH. In this way, two populations were created, one containing (endo) lysosomes, one containing the labelled VHHs. Applying the ‘intersect’ command allowed us to determine the fraction of VHH that localized inside the (endo) lysosomes, and the fraction of lysosomes that contains VHH. The total volume of analyzed cells was also measured to correct for. The calculation of the fractions was done based on the segmented volumes and volumes were expressed in ‘voxels’. A voxel is the 3D version of a pixel, so a volumetric unit in the image stack. For the live-cell imaging results, uptake per cell volume was calculated by dividing the sum of voxel count for each fluorescent VHH time point by the sum of voxel count per cell (representing the cell volume) at that time point. The percentages of VHH colocalising with lysosomes and the percentage of the entire endolysosomal pool containing the particular VHH were calculated by taking the ratio of the voxel counts of VHH-signal colocalising with LTR and of the total intracellular VHH signal. The percentage of lysosomes with VHHs was determined by the voxel count ratio of the VHH-signal colocalising with LTR and the total LTR signal. The last graph shows the absolute voxel counts of the intracellular VHH signal and the VHH-LTR colocalising signal.

### Size-Exclusion Chromatography Coupled to Multi-Angle Light Scattering

To estimate the molecular mass and stoichiometry of the hCI-M6PR_{D1-D3} and anti-CI-M6PR VHH8 protein complex, we incubated both proteins in a 1:1 and 1:3 molar fashion in HBS buffer (50 mM HEPES, 150 mM NaCl, pH7.5, 0.1 μm filtered) containing sodium azide (0.02%). The total concentration of both samples was 0.81 and 1.08 mg/ml. After SEC (Superdex 200 increase HR 10/30), eluted proteins were detected with an online UV detector (Generic UV), a mini DAWN 8 (Wyatt) multi-angle laser light scattering (MALLS) detector and an Optilab refractive index (RI) instrument (Wyatt) at 298 K. The RI increment value (dn/dc value) at 298 K and 658 nm was calculated for the determination of the protein concentration and molecular mass (dn/dc: 0.1850 ml/g). Eluted fractions between 14-40 minutes (at 0.5 mL/min) were collected for analysis on SDS-PAGE. Data analyses and reporting was performed using the ASTRA 7.3.2 software.

### Intact Mass Spectrometry of Anti-CI-M6PR VHH: Receptor Complexes

Intact proteins were separated on an Ultimate 3000 HPLC system (Thermo Fisher Scientific, Bremen, Germany) online connected to an LTQ Orbitrap XL mass spectrometer (Thermo Fischer Scientific). Briefly, approximately 4 μg of protein was injected on a Zorbax Poroshell 300SB-C8 column (5 μm, 300 Å, 1×75 mm IDxL; Agilent Technologies) and separated using a 15 min gradient from 5% to 80% solvent B at a flow rate of 100 μl/min (solvent A: 0.1% formic acid and 0.05% trifluoroacetic acid in water; solvent B: 0.1% formic acid and 0.05% trifluoroacetic acid in acetonitrile). The column temperature was maintained at 60° C. Eluting proteins were directly sprayed in the mass spectrometer with an ESI source using the following parameters: spray voltage of 4.2 kV, surface-induced dissociation of 30 V, capillary temperature of 325° C., capillary voltage of 35 V and a sheath gas flow rate of 7 (arbitrary units). The mass spectrometer was operated in MS1 mode using the orbitrap analyzer at a resolution of 100,000 (at m/z 400) and a mass range of 600-4000 m/z, in profile mode. The resulting MS spectra were deconvoluted with the BioPharma Finder 3.0 software (Thermo Fisher Scientific) using the Xtract deconvolution algorithm (isotopically resolved spectra), after which the deconvoluted spectra were annotated automatically using the BioPharma Finder protein sequence manager and protein identification tool.

### Co-Crystallisation of the VHH-hCI-M6PRD1-D3 Complex

Complexes of hCI-M6PR_{D1-D3} with either VHH7, -8 or 1H11 were formed and polished on SEC in HBS buffer (20 mM HEPES, 150 mM NaCl, pH 7.5). To this end, solutions containing hCI-M6PR_{D1-D3} and a 1.25 molar excess of either anti-CI-M6PR VHH were injected onto a Superdex 200. The fractions containing VHH complexes were collected, supplemented with mannose-6-phosphate (1 mM, M3655-100 MG, Sigma) and concentrated up to 3.5 mg/ml and 7 mg/ml respectively using an Amicon Ultra-15 protein concentrator (UFC903024, Millipore). VHH 1H11 complexes where concentrated to 3.7 mg/ml without the addition of mannose-6-phosphate. For crystalization of the complexes in general, nanolitre-scale sitting drop vapour diffusion crystallization experiments were set up at 287 K using commercially available sparse matrix crystals screens (Molecular Dimensions, Hampton Research) and a Mosquito crystallization robot (TTP Labtech). Promising hits were further optimized using gradient optimization in 96-well.

Two crystal forms of VHH7: hCI-M6PR_{D1-D3} were identified: a rhombohedral crystal, diffracting to 2.2 Å, crystallised from 0.3 M KBr, 0.1 M NaCacodylate pH 6.5, 8% w/v y-PGA (Na+ form, LM) (PGA screen condition C9: Hu et al. Acta Crystallogr D Biol Crystallogr. 2008; 64:957-63) and a tetragonal crystal form, diffracting to 3.0 Å, crystallised from 0.2 M NH₄NO₃, 0.1 M Bis-Tris propane pH 8.5, 18% v/v PEG Smear High (BCS screen condition F6). A single crystal form of hCI-M6PR_{D1-D3}: VHH8 was identified growing from sodium acetate trihydrate (0.08 M), sodium chloride (0.15 M), Tris (0.1 M), PEG Smear (0.015% v/v), pH 8) (BCS screen condition F3) which diffracted to 2.75 Å. Two crystal forms of VHH 1H11: hCI-M6PRD1-D3 were identified: a poorly diffracting rhombohedral crystal form crystallized from a few conditions amongst which 0.2 M (NH4) 2SO4 0.1 M Sodium acetate 4.6 25% v/v PEG Smear Broad (BCS screen condition C10) and a tetragonal crystal form, diffracting to 2.7 Å, crystallized from a few conditions amongst which 0.1 M Ammonium sulfate, 0.1 M Tris pH 7.5, 20% w/v PEG 1500 (Proplex screen condition A7).

The crystals containing complexes of VHH7 and VHH8 grown from BCS conditions were cryoprotected in mother liquor supplemented with ZW221 cryosolution (17.5% v/v) (Sanchez, et al. Biochemistry 54, no. 21 (2015): 3360-3369) consisting of DMSO (40%), ethylene glycol (20%) and glycerol (40%). The crystal grown from the PGA condition was cryoprotected in mother liquor supplemented with glycerol (17.5% v/v) and the crystal containing the VHH 1H11 complex was cryoprotected in mother liquor supplemented with 17.5% (v/v) ethylene glycol prior to vitrification in liquid nitrogen. Final X-ray diffraction measurements of VHH8-hCI-M6PR_{D1-D3} crystals were performed at EMBL P14 beamline (Petra 3 synchrotron, Germany), Proxima PX1 beamline (Soleil synchrotron, France) for the VHH7-hCI-M6PR_{D1-D3} crystal and ESRF ID30A3 for the VHH 1H11-hCI-M6PR_{D1-D3} crystal. All datasets originate from individual crystals. Diffraction data integration and scaling was performed in XDS. Dataset statistics are reported in Table 11. Initial phases were recovered by maximum-likelihood based molecular replacement as implemented in Phaser using CIMPR_{D1D2}, CI-M6PR_{D3} based on the bovine CIMPR structure (PDB: 1sz0) and a VHH. Structures were iteratively built and refined in Coot, Isolde (Croll, Tristan Ian. 2018. “ISOLDE: A Physically Realistic Environment for Model Building into Low-Resolution Electron-Density Maps.” Acta Crystallographica Section D: Structural Biology 74 (6): 519-30) implemented in ChimeraX and Phenix refine.

TABLE 11
Crystallographic data collection and refinement statistics. Values in parenthesis refer to highest resolution shell.

| | CIMPR VHH7 | | CIMPR VHH7 | | CIMPR VHH1H11 | | CIMPR VHH8 | |
|---|---|---|---|---|---|---|---|---|
| Collection Date | 15 Dec. 2020 | | 15 Dec. 2020 | | 30 Jan. 2022 | | 11 Sep. 2020 | |
| Synchrotron | EMBL P14 | | EMBL P14 | | ESRF ID30A3 | | Soleil PX1 | |
| Condition | PGA C9 (0.3M | | BCS F6 (0.2M | | Proplex A7 (0.1M | | BCS F3 (75 mM Na | |
| | KBr, 0.1M NaCaco | | Ammonium | | Ammonium sulfate, | | acetate, 0.15M NaCl, | |
| | pH 6.5, 8% w/v Y- | | nitrate, 0.1M | | 0.1M Tris pH 7.5, | | 0.1M Tris pH 8.0, | |
| | PGA (Na+ form, LM)) | | Bis-Tris propane | | 20% w/v PEG 1500) | | 15% (v/v) PEG Smear | |
| | | | pH 8.5, 18% v/v | | | | Medium) | |
| | | | PEG Smear High) | | | | | |
| Cryo | 17,5% Glycerol | | 17% ZW221 | | 17.5% Ethglyc | | 17% ZW221 | |
| Unit Cell | 129.09 129.09 | | 135.63 135.63 | | 126.18 126.18 | | 170.17 182.89 | |
| Parameters | 569.16 | | 95.57 | | 105.07 | | 110.32 | |
| | 90 90 120 | | 90 90 90 | | 90 90 90 | | 90 90 90 | |
| Space group | R 3 2 (no 155) | | P 4₁ 2 2 (no 91) | | P 4₁ 2₁ 2 (no 92) | | C 2 2 2₁ (no 20) | |
| Wavelength (Å) | 0.9763 | | 0.9763 | | 0.9677 | | 0.978565 | |
| Resolution (Å) | 189.72-2.2 | (2.33-2.20) | 95.91-3.00 | (3.18-3.00) | 56.43 -2.70 | (2.86-2.70) | 124.58-2.75 | (2.91-2.75) |
| Reflection | 1015492 | (123223) | 492251 | (78908) | 72465 | (11789) | 552471 | (88164) |
| observed | | | | | | | | |
| unique | 92391 | (14175) | 18434 | (2910) | 23537 | (3745) | 45143 | (7153) |
| mulitplicity | 10.99 | (8.69) | 6.24 | (6.33) | 3.08 | (3.15) | 12.24 | (12.33) |
| completeness | 99.2 | (95.0) | 100.0 | (99.9) | 98.3 | (99.4) | 99.8 | (98.7) |
| I/σ(I) | 15.98 | (1.09) | 10.94 | (1.02) | 4.96 | (0.99) | 12.58 | (1.29) |
| R-meas (%) | 9.7 | (177.1) | 39.1 | (382.3) | 21.1 | (134.5) | 18.9 | (183.9) |
| CC(½) | 99.9 | (36.9) | 99.8 | (41.0) | 98.2 | (45.9) | 99.8 | (53.3) |
| Wilson B (Å²) | 59.48 | | 72.8 | | 53.4 | | 64.5 | |
| Reflections used in | 92080 | (8194) | 18401 | (1790) | 23484 | (2337) | 45041 | (4463) |
| refinement | | | | | | | | |
| for R-free | 1996 | (180) | 1841 | (179) | 1996 | (197) | 2238 | (206) |
| R-work | 0.2058 | (0.3039) | 0.2187 | (0.3333) | 0.2379 | (0.3915) | 0.2134 | (0.3254) |
| R-free | 0.2325 | (0.3094) | 0.2578 | (0.3762) | 0.2813 | (0.4425) | 0.2563 | (0.3446) |

| | | | | |
|---|---|---|---|---|
| Number of non- | 9315 | 4225 | 4202 | 8671 |
| hydrogen atoms | | | | |
| macromolecules | 8856 | 4155 | 4107 | 8452 |
| ligands | 222 | 63 | 81 | 202 |
| solvent | 237 | 7 | 14 | 17 |
| Protein residues | 1112 | 532 | 525 | 1086 |
| RMS(bonds Å) | 0.002 | 0.012 | 0.003 | 0.003 |
| RMS(angles °) | 0.55 | 1.82 | 0.6 | 0.61 |
| Ramachandran | 96.08 | 95.04 | 95.36 | 94.69 |
| favored (%) | | | | |
| allowed (%) | 3.55 | 4.96 | 4.64 | 5.12 |
| outliers (%) | 0.36 | 0 | 0 | 0.19 |
| Rotamer | 1.33 | 1.73 | 1.1 | 1.07 |
| outliers (%) | | | | |
| Clashscore | 2.2 | 0.12 | 4.04 | 0.71 |
| Average B-factor | 82 | 92.55 | 67.83 | 79.23 |
| macromolecules | 81.36 | 92.79 | 67.47 | 79.13 |
| ligands | 128.24 | 80.81 | 90.53 | 85.37 |
| solvent | 62.67 | 57.43 | 42.09 | 55.47 |
| Number of TLS | 8 | 9 | 4 | 8 |
| groups | | | | |

### SEQUENCE LISTING

SEQ ID NO: 1-11: Amino acid sequence of CI-M6PR-specific VHH 1-VHH11.
SEQ ID NO:12: monovalent EGFR-specific VHH 9G8 (construct 14)
SEQ ID NO:13: bispecific 9G8-VHH8 fusion protein (Construct 15)
SEQ ID NO: 14: trivalent bispecific 9G8-9G8-VHH8 fusion protein (Construct 16)
SEQ ID NO: 15: bispecific 9G8-GBP fusion protein (Construct 17)
SEQ ID NO:16: trivalent bispecific 9G8-9G8-GBP fusion protein (Construct 18)
SEQ ID NO: 17: monovalent VHH 9G8 S54A (Construct 25/33)
SEQ ID NO: 18: Bispecific 9G8 S54A-VHH8 fusion protein (Construct 26/35)
SEQ ID NO: 19: Trivalent bispecific 9G8 S54A-9G8 S54A-VHH8 fusion protein (construct 27/37)
SEQ ID NO: 20: bispecific 9G8 S54A-GBP fusion protein (Construct 28/38)
SEQ ID NO: 21: Trivalent bispecific 9G8 S54A-9G8 S54A-GBP fusion protein (Construct 29/39)
SEQ ID NO:22: FLAG3His₆-tag C-terminally fused to bispecific VHHs as used in the constructs
SEQ ID NO:23: Amino acid sequence of human cation-independent mannose-6-phosphate receptor precursor [NP_000867.2; 2491aa]
SEQ ID NO:24: amino acid sequence of VHH1H11
SEQ ID NO:25: amino acid sequence of VHH1H52
SEQ ID NO: 26: amino acid sequence of humanized variant VHH7h1
SEQ ID NO:27: amino acid sequence of humanized variant VHH7h2
SEQ ID NO: 28: amino acid sequence of humanized variant VHH7h3
SEQ ID NO: 29: amino acid sequence of humanized variant VHH7hWN
SEQ ID NO:30: amino acid sequence of humanized variant VHH8h1
SEQ ID NO:31: amino acid sequence of humanized variant VHH8h2
SEQ ID NO:32: amino acid sequence of humanized variant VHH8h3
SEQ ID NO:33: amino acid sequence of humanized variant VHH8h4
SEQ ID NO:34: amino acid sequence of humanized variant VHH8h5
SEQ ID NO:35: amino acid sequence of humanized variant VHH8hWN

TABLE 12
CDR sequences of CI-M6PR VHHs binding to the epitope of VHH7 or VHH8, wherein CDRs
are annotated according to AbM

| | SEQ | | SEQ ID | | SEQ ID | | SEQ ID |
|---|---|---|---|---|---|---|---|
| VHH | ID NO: | CDR1 | NO: | CDR2 | NO: | CDR3 | NO: |
| VHH7 | 7 | GIIFSDNRMD | 36 | TLASYGWKT | 42 | SSPVLNDI | 48 |
| VHH1 | 1 | GFTFDRYWMN | 37 | TINTGGTGTY | 43 | GATYYRGNSAI | 49 |
| VHH1H11 | 24 | GIIFSDNRMD | 38 | TLASYGWKT | 44 | NSGQY | 50 |
| VHH8 | 8 | GRTFNTYNWG | 39 | AIRWSSSKTS | 45 | SIVDFTTNPSHFGS | 51 |
| VHH5 | 5 | GRTFSRLAMG | 40 | AISENGDSIH | 46 | DRAAYYCSGSGCYPSRAPA | 52 |
| | | | | | | AASYDY | |
| VHH1H52 | 25 | GFTWDSYVIG | 41 | CLDVDDGSIY | 47 | VNRASMRFRRCLQVLRYDY | 53 |

TABLE 13
FR sequences of CI-M6PR VHHs binding to the epitope of VHH7 or VHH8, wherein CDRs are
annotated according to AbM

| | FL | | | | | | | | |
|---|---|---|---|---|---|---|---|---|---|
| | SEQ | | SEQ | | SEQ | | SEQ | | |
| | ID | | ID | | ID | | ID | | SEQ |
| VHH | NO: | FR1 | NO: | FR2 | NO: | FR3 | NO: | FR4 | ID NO: |
| VHH7 | 7 | QVQLQESGGGLV | 54 | WYRQAPGK | 60 | YADSVKGRFTISR | 66 | WGQGTQ | 72 |
| | | QPGGSLRLSCAAS | | QREWVA | | DNTKNTVYLRM | | VTVSS | |
| | | | | | | NSLKPEDTAVYY | | | |
| | | | | | | CKA | | | |
| VHH1 | 1 | QVQLQESGGGLV | 55 | WLRQIPGKE | 61 | YDDSVKGRFSISR | 67 | RGQGTQV | 73 |
| | | QPGGSLRLSCVAS | | IEWVS | | DDAKNTLYLTM | | TVSS | |
| | | | | | | NSLKTEDTAVYY | | | |
| | | | | | | CAR | | | |
| VHH1H11 | 24 | QVQLQESGGGLV | 56 | WYRQAPGK | 62 | YADSVKDRFTISR | 68 | WGQGTQ | 74 |
| | | QPGGSLRLSCAAS | | QREWVA | | DNAKNTVNLYM | | VTVSS | |
| | | | | | | NSLQPEDTAVYY | | | |
| | | | | | | C | | | |
| VHH8 | 8 | QVQLQESGGGLV | 57 | WFRQAPGK | 63 | YADSVKGRFTISR | 69 | WGQGTQ | 75 |
| | | QAGGSLRLSCEAS | | EREFVA | | DNAKNTIYLQM | | VTVSS | |
| | | | | | | NSLKPEDTAVYY | | | |
| | | | | | | CAA | | | |
| VHH5 | 5 | QVQLQESGGGLV | 58 | WFRQAPGK | 64 | YSDSVKGRFAVS | 70 | WGQGTQ | 76 |
| | | QAGGSLKLSCAAA | | EREFVA | | RDNAKNTVYLQ | | VTVSS | |
| | | | | | | MNSLKPEDTAIY | | | |
| | | | | | | YCAA | | | |
| VHH1H52 | 25 | QVQLQESGGGLV | 59 | WFRQAPGK | 65 | YHDSAKGRFSISR | 71 | WGQGTQ | 77 |
| | | QPGGSLRLSCTAS | | GREGVS | | DNAKNTVYLQM | | VTVSS | |
| | | | | | | NSLKPEDTAVYY | | | |
| | | | | | | CAA | | | |

SEQ ID NO:78: FR1 consensus (including humanization)
SEQ ID NO:79: FR2 consensus sequence (including humanization)
SEQ ID NO:80: FR3 consensus sequence (including humanization)
SEQ ID NO:81: FR4 consensus sequence (including humanization)
SEQ ID NO:82: Bispecific 9G8 S54A-VHH7 fusion protein (construct 34)
SEQ ID NO:83: Trivalent bispecific 9G8 S54A-9G8 S54A-VHH7 fusion protein (construct 36)
SEQ ID NO:84: Bispecific 9G8 S54A-VHH7 fusion protein (construct 40)
SEQ ID NO:85: Bispecific 9G8 S54A-VHH8 fusion protein (construct 41)
SEQ ID NO:86: Light chain of Cetuximab
SEQ ID NO:87: Heavy chain of Cetuximab
SEQ ID NO: 88: Heavy chain of Cetuximab-VHH7
SEQ ID NO:89: Heavy chain of Cetuximab-VHH8
SEQ ID NO:90: Monovalent GBP (construct 42)
SEQ ID NO:91: Bispecific GBP-VHH7 fusion protein (construct 43)
SEQ ID NO:92: Bispecific GBP-VHH8 fusion protein (construct 44)
SEQ ID NO:93: Bispecific GBP-VHH1 fusion protein (construct 45)
SEQ ID NO:94: Bispecific GBP-VHH5 fusion protein (construct 46)
SEQ ID NO:95: Bispecific GBP-1H11 fusion protein (construct 47)
SEQ ID NO:96: Bispecific GBP-1H52 fusion protein (construct 48)
SEQ ID NO:97: Amino acid sequence of mouse cation-independent mannose-6-phosphate receptor precursor [NP_034645.2; 2483 aa]
SEQ ID NO:98: Amino acid sequence of bovine cation-independent mannose-6-phosphate receptor precursor [NP_776777.1; 2499 aa]

### REFERENCES

1. Hamers-Casterman, C. et al. Nature 363, 446-448 (1993).
2. De Duve C, Pressman B C, Gianetto R, Wattiaux R, Appelmans F: Tissue fractionation studies. 6. Intracellular distribution patterns of enzymes in rat-liver tissue. Biochem J 1955, 60:604-617.
3. Muyldermans, S. Annu. Rev. Biochem. 82, 775-797 (2013).
4. Schrankel, C. S., Gökirmak, T., Lee, C.-W., Chang, G. & Hamdoun, A. Methods Cell Biol. 151, 353-376 (2019).
5. Laeremans, T., Haard, H. D. & Hoogenboom, H. R. J. M. (2007)—WO2007042289A2
6. Roovers, R. C. et al. Cancer Immunol. Immunother. 56,303-317 (2007).
7. York, S. J. et al. J. Biol. Chem. 274, 1164-1171 (1999).
8. Wang, Q., Villeneuve, G. & Wang, Z. EMBO Rep. 6, 942-948 (2005).
9. Jovčevska, I. & Muyldermans, S. BioDrugs 34, 11-26 (2020).
10. Banik S M, Pedram K, Wisnovsky S, Ahn G, Riley N M, Bertozzi C R: Lysosome-targeting chimaeras for degradation of extracellular proteins. Nature 2020, 584:291-297.
11. Lin-Cereghino J, Wong W W, Xiong S, Giang W, Luong L T, Vu J, Johnson S D, Lin-Cereghino G P: Condensed protocol for competent cell preparation and transformation of the methylotrophic yeast Pichia pastoris. Biotechniques 2005, 38:44-48.
12. Sand, K. M. K. et al. Front. Immunol. 5, (2015).
13. Yang J, Zhang Y: I-TASSER server: New development for protein structure and function predictions. Nucleic Acids Research 2015, 43: W174-W181.
14. Schrödinger, LLC: The {PyMOL} Molecular Graphics System, Version˜1.8. 2015.
15. Ahn et al. Nature Chemical Biology, Vol. 17; 2021; p. 937-946
16. Camacho, C. J. & Zhang, C. FastContact: rapid estimate of contact and binding free energies. Bioinforma. Oxf. Engl. 21, 2534-2536 (2005).
17. Olson U J, Dahms N M, Kim J-J P: The N-terminal Carbohydrate Recognition Site of the Cation-independent Mannose 6-Phosphate Receptor. J Biol Chem 2004, 279:34000-34009.
18. E. Krissinel, K. Henrick, J. Mol. Biol. 372, 774-797 (2007).
19. Pardon, et al. A general protocol for the generation of Nanobodies for structural biology. Nature Protocols 2014. 9:674-693.
20. Borden, L. A., Einstein, R., Gabel, C. A. & Maxfield, F. R. J. Biol. Chem. 1990. 265, 8497-8504.
21. Epenetos, A. A., Snook, D., Durbin, H., Johnson, P. M. & Taylor-Papadimitriou, J. Cancer Res. 1986. 46, 3183-3191.
22. Schröter, C. et al. mAbs 7, 138-151 (2014).
23. Pabla, B., Bissonnette, M. & Konda, V. J. World J. Clin. Oncol. 6, 133-141 (2015).
24. Bokemeyer, C. et al. Eur. J. Cancer Oxf. Engl. 1990 48, 1466-1475 (2012).
25. Tan, C. et al. World J. Gastroenterol. WJG 18, 5171-5180 (2012).
26. Di Nicolantonio, F. et al. J. Clin. Oncol. Off. J. Am. Soc. Clin. Oncol. 26, 5705-12 (2008).
27. Cunningham, D. et al. N. Engl. J. Med. 351, 337-345 (2004).
28. Arena, S. et al. Clin. Cancer Res. 21, 2157-2166 (2015).
29. Katreddy, R. R. et al. Oncogenesis 7, 5 (2018).
30. Weihua, Z. et al. Cancer Cell 13, 385-393 (2008).

1. A protein binding agent comprising:
an immunoglobulin-single-variable domain (ISVD) which specifically binds human cation-independent mannose-6-phosphate receptor (CI-M6PR) on the extracellular N-terminal CI-M6PR domains 1, 2 and/or 3, wherein the ISVD is fused to a binding agent which specifically binds an extracellularly-accessible protein target.

2. The protein binding agent of claim 1, wherein the CI-M6PR-specific ISVD specifically binds an epitope comprising the amino acid residues Lys191, Gly194, Ala195, Tyr196, Leu197, Phe208, Arg219, Gln224, Leu225, Ile297, Lys357, Gly408, Asp409, Asn431, Glu433, and Phe457 or an epitope comprising the amino acid residues Lys59, Asn60, Met85, Asp87, Lys89, Ala146, Thr147, and Glu148, and Asp118 or Gln119, as set forth in SEQ ID NO:23.

3. The protein binding agent of claim 1, wherein the CI-M6PR-specific ISVD specifically binds to CI-M6PR via the paratope comprising residues 32, 52-57, 100-103, 108 as set forth in SEQ ID NO:8, or via the paratope comprising residues 31, 33, 35, 53, 54, 56, 57, 96, 104 as set forth in SEQ ID NO:7, or the paratope comprising residues 31-35, 50, 52-57, 96-98 as set forth in SEQ ID NO:24.

4. The protein binding agent of claim 1, wherein the CI-M6PR-specific ISVD comprises 4 framework regions (FR) and 3 complementarity-determining regions (CDR) according to the following formula (1): FR1-CDR1-FR2-CDR2-FR3-CDR3-FR4 (1), and the CDR1, CDR2 and CDR3 regions are selected from those CDR1, CDR2 and CDR3 regions of a sequence selected from the group of sequences of SEQ ID NO: 1, 5, 7, 8, 24, or 25, wherein the CDR regions are annotated according to Kabat, MacCallum, IMGT, AbM, or Chothia.

5. The protein binding agent of claim 1, wherein the CI-M6PR-specific ISVD comprises a CDR1 sequence selected from SEQ ID NO: 36-41, a CDR2 sequence selected from SEQ ID NO: 42-47, and a CDR3 sequence selected from SEQ ID NO: 48-53.

6. The protein binding agent of claim 5, wherein the CI-M6PR-specific ISVD agent comprises a FR1 sequence corresponding to SEQ ID NO: 78, a FR2 sequence corresponding to SEQ ID NO: 79, a FR3 sequence corresponding to SEQ ID NO: 80, and a FR4 sequence corresponding to SEQ ID NO: 81.

7. The protein binding agent of claim 1, wherein the protein binding agent comprises a CI-M6PR-specific ISVD comprising a sequence selected from the group of sequences of SEQ ID NO: 1, 5, 7, 8, 24, or 25, or a sequence with at least 85% amino acid identity thereof wherein the CDRs are identical, a humanized variant thereof, or anyone of SEQ ID NOs: 26-35.

8. The protein binding agent of claim 1, wherein the CI-M6PR-specific ISVD and binding agent specifically binding an extracellularly-accessible protein target are fused by a linker, a short peptide linker, a glycine-serine linker, an Fc-tail, or another moiety.

9. The protein binding agent of claim 1, wherein the binding agent specifically binding the extracellularly-accessible protein comprises an antigen-binding protein domain, an ISVD, a VHH-Fc fusion, a VHH-Fc-VHH, a knob-into-hole VHH-Fc fusion, an antibody, such as or an IgG.

10. The protein binding agent of claim 1, wherein the protein binding agent is a multi-specific or multivalent binding agent comprising the CI-M6PR-specific ISVD fused to the extracellularly-accessible protein-specific binding agent and a further; functional moiety, a therapeutic moiety, antigen-binding domain, or a half-life extension moiety.

11. The protein binding agent of claim 1, which further comprises a detectable label or a tag.

12. The protein binding agent of claim 1, wherein the extracellularly-accessible protein target is the epidermal growth factor receptor (EGFR), and/or the EGFR-specific binding agent comprises SEQ ID NO: 12 or SEQ ID NO: 17, or a homologue with at least 90% identity thereof wherein the CDRs are identical, or comprises SEQ ID NO: 86 and 87.

13. The protein binding agent of claim 12, wherein the protein binding agent comprises a sequence selected from the group of sequences of SEQ ID NOs: 13, 14, 18, 19, 82 to 85, or a functional homologue with at least 90% identity thereof wherein the CDRs are identical, or the antibody formed by the heavy chain-ISVD fusion of SEQ ID NO: 88 or 89 and light chain of SEQ ID NO: 86.

14. A nucleic acid molecule encoding the protein binding agent of claim 1.

15. (canceled)

16. The protein binding agent of claim 1, wherein the protein binding agent is comprised in a pharmaceutical composition or a medicament.

17. (canceled)

18. (canceled)

19. (canceled)

20. (canceled)

21. A method for removing a cell surface molecule and/or degrading the molecule in the lysosome, the method comprising contacting a cell comprising the cell surface molecule with the protein binding agent of claim 1.

22. (canceled)

23. (canceled)


SEQUENCE LISTING (98 sequences)

SEQ ID NO: 1 - protein, 120 aa, synthetic construct
QVQLQESGGGLVQPGGSLRLSCVASGFTFDRYWMNWLRQIPGKEIEWVSTINTGGTGTYYDDSVKGRFSISRDDAKNTLYLTMNSLKTEDTAVYYCARGATYYRGNSAIRGQGTQVTVSS

SEQ ID NO: 2 - protein, 118 aa, synthetic construct
QVQLQESGGGLVQPGGSLRLSCVFSGRTFMSYDVGWFRQAPGKERELLSSITSSSSSPYYAESVKGRFAVSRDGAKKTVYLRMNSLEPEDTAVYYCAARENWRNDWQYWGQTQVTVSS

SEQ ID NO: 3 - protein, 119 aa, synthetic construct
QVQLQESGGGLVQPGGSLRLSCAAFGLNFKNFDMIWFRQAPGEERDIVAAISSSSSTIWYEDSVKGRFTISRDNAKNTVYLQMNSLKPEDTAVYYCAAALTSWRRPDYWGQGTQVTVSS

SEQ ID NO: 4 - protein, 123 aa, synthetic construct
QVQLQESGGGLVQAGGSLRLSCAASGRTFSNYVMGWFRQAPGKEREFVGSISQGGKMTYYPDSVKGRFTISRDNAKNTMYLQMDGLKPEDTAVYYCAVSRGYPMVRVSEVDYWGQGTQVTVSS

SEQ ID NO: 5 - protein, 134 aa, synthetic construct
QVQLQESGGGLVQAGGSLKLSCAAAGRTFSRLAMGWFRQAPGKEREFVAAISENGDSIHYSDSVKGRFAVSRDNAKNTVYLQMNSLKPEDTAIYYCAADRAAYYCSGSGCYPSRAPAAASYDYWGQGTQVTVSS

SEQ ID NO: 6 - protein, 112 aa, synthetic construct
QVQLQESGGGLVQAGGSLRLSCAVSGSISSIGGMGWFRQAPGKQREFVAVILNNSGMTSYADSVKGRFTISRGNSKNTMYLQMNSLKPEDTAVYYCNYSGYWGQGTQVTVSS

SEQ ID NO: 7 - protein, 116 aa, synthetic construct
QVQLQESGGGLVQPGGSLRLSCAASGIIFSDNRMDWYRQAPGKQREWVATLASYGWKTYADSVKGRFTISRDNTKNTVYLRMNSLKPEDTAVYYCKASSPVLNDIWGQGTQVTVSS

SEQ ID NO: 8 - protein, 123 aa, synthetic construct
QVQLQESGGGLVQAGGSLRLSCEASGRTFNTYNWGWFRQAPGKEREFVAAIRWSSSKTSYADSVKGRFTISRDNAKNTIYLQMNSLKPEDTAVYYCAASIVDFTTNPSHFGSWGQGTQVTVSS

SEQ ID NO: 9 - protein, 113 aa, synthetic construct
QVQLQESGGGLVQAGGSLRLSCAASGTILSIKTMGWYRQAPGKEREWVATITSSSIPMYADSVKGRFTISRDDAKNTVYLQMNSLKPEDTGVYYCNAPWTDYWGQGTQVTVSS

SEQ ID NO: 10 - protein, 127 aa, synthetic construct
QVQLQESGGGSVQAGGSLRLSCAPAGRTFSRIRMGWFRQAPGKEREFVAAISSGGLSYYADSMKGRFTISRDNAKNTVLLQMNSLEPDDTAVYYCAAVLNDYIDDYVARNPTLYDYWGQGTQVTVSS

SEQ ID NO: 11 - protein, 124 aa, synthetic construct
QVQLQESGGGLVQAGDSLKLSCTASGPTFSEITGGWFRQVPGKEREFVASISSTGITTYYTDSVKGRFTVSRDNAKNTMYLQMNSLKPEDTGVYYCAARSTSRSIAITVGYDYWGQGTQVTVSS

SEQ ID NO: 12 - protein, 127 aa, synthetic construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSS

SEQ ID NO: 13 - protein, 267 aa, synthetic construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGSGGGGSGGGGSGGGGSQVQLQESGGGLVQAGGSLRLSCEASGRTFNTYNWGWFRQAPGKEREFVAAIRWSSSKTSYADSVKGRFTISRDNAKNTIYLQMNSLKPEDTAVYYCAASIVDFTTNPSHFGSWGQGTQVTVSS

SEQ ID NO: 14 - protein, 409 aa, synthetic construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGGSGGGGSEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGSGGGGSGGGGSGGGGSQVQLQESGGGLVQAGGSLRLSCEASGRTFNTYNWGWFRQAPGKEREFVAAIRWSSSKTSYADSVKGRFTISRDNAKNTIYLQMNSLKPEDTAVYYCAASIVDFTTNPSHFGSWGQGTQVTVSS

SEQ ID NO: 15 - protein, 259 aa, synthetic construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGSGGGGSGGGGSGGGGSQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 16 - protein, 401 aa, synthetic construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGGSGGGGSEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGSGGGGSGGGGSGGGGSQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 17 - protein, 127 aa, synthetic construct
EVQLVESGGGLVQAGGSLRLSCAASGRTFSSYAMGWFRQAPGKEREFVVAINWASGSTYYADSVKGRFTISRDNAKNTMYLQMNSLKPEDTAVYYCAAGYQINSGNYNFKDYEYDYWGQGTQVTVSS

SEQ ID NO: 18 - protein, 267 aa, synthetic construct
EVQLVESGGGLVQAGGSLRLSCAASGRTFSSYAMGWFRQAPGKEREFVVAINWASGSTYYADSVKGRFTISRDNAKNTMYLQMNSLKPEDTAVYYCAAGYQINSGNYNFKDYEYDYWGQGTQVTVSSGSGGGGSGGGGSGGGGSQVQLQESGGGLVQAGGSLRLSCEASGRTFNTYNWGWFRQAPGKEREFVAAIRWSSSKTSYADSVKGRFTISRDNAKNTIYLQMNSLKPEDTAVYYCAASIVDFTTNPSHFGSWGQGTQVTVSS

SEQ ID NO: 19 - protein, 409 aa, synthetic construct
EVQLVESGGGLVQAGGSLRLSCAASGRTFSSYAMGWFRQAPGKEREFVVAINWASGSTYYADSVKGRFTISRDNAKNTMYLQMNSLKPEDTAVYYCAAGYQINSGNYNFKDYEYDYWGQGTQVTVSSGGGGSGGGGSGGGGSEVQLVESGGGLVQAGGSLRLSCAASGRTFSSYAMGWFRQAPGKEREFVVAINWASGSTYYADSVKGRFTISRDNAKNTMYLQMNSLKPEDTAVYYCAAGYQINSGNYNFKDYEYDYWGQGTQVTVSSGSGGGGSGGGGSGGGGSQVQLQESGGGLVQAGGSLRLSCEASGRTFNTYNWGWFRQAPGKEREFVAAIRWSSSKTSYADSVKGRFTISRDNAKNTIYLQMNSLKPEDTAVYYCAASIVDFTTNPSHFGSWGQGTQVTVSS

SEQ ID NO: 20 - protein, 259 aa, synthetic construct
EVQLVESGGGLVQAGGSLRLSCAASGRTFSSYAMGWFRQAPGKEREFVVAINWASGSTYYADSVKGRFTISRDNAKNTMYLQMNSLKPEDTAVYYCAAGYQINSGNYNFKDYEYDYWGQGTQVTVSSGSGGGGSGGGGSGGGGSQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 21 - protein, 401 aa, synthetic construct
EVQLVESGGGLVQAGGSLRLSCAASGRTFSSYAMGWFRQAPGKEREFVVAINWSAGSTYYADSVKGRFTISRDNAKNTMYLQMNSLKPEDTAVYYCAAGYQINSGNYNFKDYEYDYWGQGTQVTVSSGGGGSGGGGSGGGGSEVQLVESGGGLVQAGGSLRLSCAASGRTFSSYAMGWFRQAPGKEREFVVAINWASGSTYYADSVKGRFTISRDNAKNTMYLQMNSLKPEDTAVYYCAAGYQINSGNYNFKDYEYDYWGQGTQVTVSSGSGGGGSGGGGSGGGGSQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 22 - protein, 30 aa, synthetic construct
DYKDDDDKDYKDDDDKDYKDDDDKHHHHHH

SEQ ID NO: 23 - protein, 2491 aa, Homo sapiens
MGAAAGRSPHLGPAPARRPQRSLLLLQLLLLVAAPGSTQAQAAPFPELCSYTWEAVDTKNNVLYKINICGSVDIVQCGPSSAVCMHDLKTRTYHSVGDSVLRSATRSLLEFNTTVSCDQQGTNHRVQSSIAFLCGKTLGTPEFVTATECVHYFEWRTTAACKKDIFKANKEVPCYVFDEELRKHDLNPLIKLSGAYLVDDSDPDTSLFINVCRDIDTLRDPGSQLRACPPGTAACLVRGHQAFDVGQPRDGLKLVRKDRLVLSYVREEAGKLDFCDGHSPAVTITFVCPSERREGTIPKLTAKSNCRYEIEWITEYACHRDYLESKTCSLSGEQQDVSIDLTPLAQSGGSSYISDGKEYLFYLNVCGETEIQFCNKKQAAVCQVKKSDTSQVKAAGRYHNQTLRYSDGDLTLIYFGGDECSSGFQRMSVINFECNKTAGNDGKGTPVFTGEVDCTYFFTWDTEYACVKEKEDLLCGATDGKKRYDLSALVRHAEPEQNWEAVDGSQTETEKKHFFINICHRVLQEGKARGCPEDAAVCAVDKNGSKNLGKFISSPMKEKGNIQLSYSDGDDCGHGKKIKTNITLVCKPGDLESAPVLRTSGEGGCFYEFEWHTAAACVLSKTEGENCTVFDSQAGFSFDLSPLTKKNGAYKVETKKYDFYINVCGPVSVSPCQPDSGACQVAKSDEKTWNLGLSNAKLSYYDGMIQLNYRGGTPYNNERHTPRATLITFLCDRDAGVGFPEYQEEDNSTYNFRWYTSYACPEEPLECVVTDPSTLEQYDLSSLAKSEGGLGGNWYAMDNSGEHVTWRKYYINVCRPLNPVPGCNRYASACQMKYEKDQGSFTEVVSISNLGMAKTGPVVEDSGSLLLEYVNGSACTTSDGRQTTYTTRIHLVCSRGRLNSHPIFSLNWECVVSFLWNTEAACPIQTTTDTDQACSIRDPNSGFVFNLNPLNSSQGYNVSGIGKIFMFNVCGTMPVCGTILGKPASGCEAETQTEELKNWKPARPVGIEKSLQLSTEGFITLTYKGPLSAKGTADAFIVRFVCNDDVYSGPLKFLHQDIDSGQGIRNTYFEFETALACVPSPVDCQVTDLAGNEYDLTGLSTVRKPWTAVDTSVDGRKRTFYLSVCNPLPYIPGCQGSAVGSCLVSEGNSWNLGVVQMSPQAAANGSLSIMYVNGDKCGNQRFSTRITFECAQISGSPAFQLQDGCEYVFIWRTVEACPVVRVEGDNCEVKDPRHGNLYDLKPLGLNDTIVSAGEYTYYFRVCGKLSSDVCPTSDKSKVVSSCQEKREPQGFHKVAGLLTQKLTYENGLLKMNFTGGDTCHKVYQRSTAIFFYCDRGTQRPVFLKETSDCSYLFEWRTQYACPPFDLTECSFKDGAGNSFDLSSLSRYSDNWEAITGTGDPEHYLINVCKSLAPQAGTEPCPPEAAACLLGGSKPVNLGRVRDGPQWRDGIIVLKYVDGDLCPDGIRKKSTTIRFTCSESQVNSRPMFISAVEDCEYTFAWPTATACPMKSNEHDDCQVTNPSTGHLFDLSSLSGRAGFTAAYSEKGLVYMSICGENENCPPGVGACFGQTRISVGKANKRLRYVDQVLQLVYKDGSPCPSKSGLSYKSVISFVCRPEAGPTNRPMLISLDKQTCTLFFSWHTPLACEQATECSVRNGSSIVDLSPLIHRTGGYEAYDESEDDASDTNPDFYINICQPLNPMHGVPCPAGAAVCKVPIDGPPIDIGRVAGPPILNPIANEIYLNFESSTPCLADKHFNYTSLIAFHCKRGVSMGTPKLLRTSECDFVFEWETPVVCPDEVRMDGCTLTDEQLLYSFNLSSLSTSTFKVTRDSRTYSVGVCTFAVGPEQGGCKDGGVCLLSGTKGASFGRLQSMKLDYRHQDEAVVLSYVNGDRCPPETDDGVPCVFPFIFNGKSYEECIIESRAKLWCSTTADYDRDHEWGFCRHSNSYRTSSIIFKCDEDEDIGRPQVFSEVRGCDVTFEWKTKVVCPPKKLECKFVQKHKTYDLRLLSSLTGSWSLVHNGVSYYINLCQKIYKGPLGCSERASICRRTTTGDVQVLGLVHTQKLGVIGDKVVVTYSKGYPCGGNKTASSVIELTCTKTVGRPAFKRFDIDSCTYYFSWDSRAACAVKPQEVQMVNGTITNPINGKSFSLGDIYFKLFRASGDMRTNGDNYLYEIQLSSITSSRNPACSGANICQVKPNDQHFSRKVGTSDKTKYYLQDGDLDVVFASSSKCGKDKTKSVSSTIFFHCDPLVEDGIPEFSHETADCQYLFSWYTSAVCPLGVGFDSENPGDDGQMHKGLSERSQAVGAVLSLLLVALTCCLLALLLYKKERRETVISKLTTCCRRSSNVSYKYSKVNKEEETDENETEWLMEEIQLPPPRQGKEGQENGHITTKSVKALSSLHGDDQDSEDEVLTIPEVKVHSGRGAGAESSHPVRNAQSNALQEREDDRVGLVRGEKARKGKSSSAQQKTVSSTKLVSFHDDSDEDLLHI

SEQ ID NO: 24 - protein, 111 aa, synthetic construct
QVQLQESGGGLVQPGGSLRLSCAASGIIFSDNRMDWYRQAPGKQREWVATLASYGWKTYADSVKDRFTISRDNAKNTVNLYMNSLQPEDTAVYYCNSGQYWGQGTQVTVSS

SEQ ID NO: 25 - protein, 128 aa, synthetic construct
QVQLQESGGGLVQPGGSLRLSCTASGFTWDSYVIGWFRQAPGKGREGVSCLDVDDGSIYYHDSAKGRFSISRDNAKNTVYLQMNSLKPEDTAVYYCAAVNRASMRFRRCLQVLRYDYWGQGTQVTVSS

SEQ ID NO: 26 - protein, 116 aa, synthetic construct
EVQLVESGGGLVQPGGSLRLSCAASGIIFSDNRMDWYRQAPGKQREWVATLASYGWKTYADSVKGRFTISRDNSKNTVYLQMNSLRPEDTAVYYCKASSPVLNDIWGQGTLVTVSS

SEQ ID NO: 27 - protein, 116 aa, synthetic construct
DVQLVESGGGLVQPGGSLRLSCAASGIIFSDNRMDWYRQAPGKQREWVATLASYGWKTYADSVKGRFTISRDNSKNTVYLQMNSLRPEDTAVYYCKASSPVLNDIWGQGTLVTVSS

SEQ ID NO: 28 - protein, 116 aa, synthetic construct
DVQLVESGGGLVQPGGSLRLSCAASGIIFSDNRMDWYRQAPGKQREWVATLASYGWKTYADSVKGRFTISRDNTKNTVYLQMNSLRPEDTAVYYCKASSPVLNDIWGQGTLVTVSS

SEQ ID NO: 29 - protein, 116 aa, synthetic construct
DVQLVESGGGLVQPGGSLRLSCAASGIIFSDNRMDWYRQAPGKQREWVATLASYGWKTYADSVKGRFTISRDNAKNTVYLQMNSLRPEDTAVYYCKASSPVLNDIWGQGTLVTVSS

SEQ ID NO: 30 - protein, 123 aa, synthetic construct
EVQLVESGGGLVQPGGSLRLSCAASGRTFNTYNWGWFRQAPGKEREFVAAIRWSSSKTSYADSVKGRFTISRDNSKNTVYLQMNSLRPEDTAVYYCAASIVDFTTNPSHFGSWGQGTLVTVSS

SEQ ID NO: 31 - protein, 123 aa, synthetic construct
DVQLVESGGGLVQPGGSLRLSCAASGRTFNTYNWGWFRQAPGKEREFVAAIRWSSSKTSYADSVKGRFTISRDNSKNTVYLQMNSLRPEDTAVYYCAASIVDFTTNPSHFGSWGQGTLVTVSS

SEQ ID NO: 32 - protein, 123 aa, synthetic construct
DVQLVESGGGLVQAGGSLRLSCAASGRTFNTYNWGWFRQAPGKEREFVAAIRWSSSKTSYADSVKGRFTISRDNSKNTVYLQMNSLRPEDTAVYYCAASIVDFTTNPSHFGSWGQGTLVTVSS

SEQ ID NO: 33 - protein, 123 aa, synthetic construct
DVQLVESGGGLVQPGGSLRLSCEASGRTFNTYNWGWFRQAPGKEREFVAAIRWSSSKTSYADSVKGRFTISRDNSKNTVYLQMNSLRPEDTAVYYCAASIVDFTTNPSHFGSWGQGTLVTVSS

SEQ ID NO: 34 - protein, 123 aa, synthetic construct
DVQLVESGGGLVQAGGSLRLSCEASGRTFNTYNWGWFRQAPGKEREFVAAIRWSSSKTSYADSVKGRFTISRDNSKNTVYLQMNSLRPEDTAVYYCAASIVDFTTNPSHFGSWGQGTLVTVSS

SEQ ID NO: 35 - protein, 123 aa, synthetic construct
DVQLVESGGGLVQPGGSLRLSCAASGRTFNTYNWGWFRQAPGKEREFVAAIRWSSSKTSYADSVKGRFTISRDNAKNTIYLQMNSLRPEDTAVYYCAASIVDFTTNPSHFGSWGQGTLVTVSS

SEQ ID NO: 36 - protein, 10 aa, synthetic construct
GIIFSDNRMD

SEQ ID NO: 37 - protein, 10 aa, synthetic construct
GFTFDRYWMN

SEQ ID NO: 38 - protein, 10 aa, synthetic construct
GIIFSDNRMD

SEQ ID NO: 39 - protein, 10 aa, synthetic construct
GRTFNTYNWG

SEQ ID NO: 40 - protein, 10 aa, synthetic construct
GRTFSRLAMG

SEQ ID NO: 41 - protein, 10 aa, synthetic construct
GFTWDSYVIG

SEQ ID NO: 42 - protein, 9 aa, synthetic construct
TLASYGWKT

SEQ ID NO: 43 - protein, 10 aa, synthetic construct
TINTGGTGTY

SEQ ID NO: 44 - protein, 9 aa, synthetic construct
TLASYGWKT

SEQ ID NO: 45 - protein, 10 aa, synthetic construct
AIRWSSSKTS

SEQ ID NO: 46 - protein, 10 aa, synthetic construct
AISENGDSIH

SEQ ID NO: 47 - protein, 10 aa, synthetic construct
CLDVDDGSIY

SEQ ID NO: 48 - protein, 8 aa, synthetic construct
SSPVLNDI

SEQ ID NO: 49 - protein, 11 aa, synthetic construct
GATYYRGNSAI

SEQ ID NO: 50 - protein, 5 aa, synthetic construct
NSGQY

SEQ ID NO: 51 - protein, 14 aa, synthetic construct
SIVDFTTNPSHFGS

SEQ ID NO: 52 - protein, 25 aa, synthetic construct
DRAAYYCSGSGCYPSRAPAAASYDY

SEQ ID NO: 53 - protein, 19 aa, synthetic construct
VNRASMRFRRCLQVLRYDY

SEQ ID NO: 54 - protein, 25 aa, synthetic construct
QVQLQESGGGLVQPGGSLRLSCAAS

SEQ ID NO: 55 - protein, 25 aa, synthetic construct
QVQLQESGGGLVQPGGSLRLSCVAS

SEQ ID NO: 56 - protein, 25 aa, synthetic construct
QVQLQESGGGLVQPGGSLRLSCAAS

SEQ ID NO: 57 - protein, 25 aa, synthetic construct
QVQLQESGGGLVQAGGSLRLSCEAS

SEQ ID NO: 58 - protein, 25 aa, synthetic construct
QVQLQESGGGLVQAGGSLKLSCAAA

SEQ ID NO: 59 - protein, 25 aa, synthetic construct
QVQLQESGGGLVQPGGSLRLSCTAS

SEQ ID NO: 60 - protein, 14 aa, synthetic construct
WYRQAPGKQREWVA

SEQ ID NO: 61 - protein, 14 aa, synthetic construct
WLRQIPGKEIEWVS

SEQ ID NO: 62 - protein, 14 aa, synthetic construct
WYRQAPGKQREWVA

SEQ ID NO: 63 - protein, 14 aa, synthetic construct
WFRQAPGKEREFVA

SEQ ID NO: 64 - protein, 14 aa, synthetic construct
WFRQAPGKEREFVA

SEQ ID NO: 65 - protein, 14 aa, synthetic construct
WFRQAPGKGREGVS

SEQ ID NO: 66 - protein, 39 aa, synthetic construct
YADSVKGRFTISRDNTKNTVYLRMNSLKPEDTAVYYCKA

SEQ ID NO: 67 - protein, 39 aa, synthetic construct
YDDSVKGRFSISRDDAKNTLYLTMNSLKTEDTAVYYCAR

SEQ ID NO: 68 - protein, 37 aa, synthetic construct
YADSVKDRFTISRDNAKNTVNLYMNSLQPEDTAVYYC

SEQ ID NO: 69 - protein, 39 aa, synthetic construct
YADSVKGRFTISRDNAKNTIYLQMNSLKPEDTAVYYCAA

SEQ ID NO: 70 - protein, 39 aa, synthetic construct
YSDSVKGRFAVSRDNAKNTVYLQMNSLKPEDTAIYYCAA

SEQ ID NO: 71 - protein, 39 aa, synthetic construct
YHDSAKGRFSISRDNAKNTVYLQMNSLKPEDTAVYYCAA

SEQ ID NO: 72 - protein, 11 aa, synthetic construct
WGQGTQVTVSS

SEQ ID NO: 73 - protein, 11 aa, synthetic construct
RGQGTQVTVSS

SEQ ID NO: 74 - protein, 11 aa, synthetic construct
WGQGTQVTVSS

SEQ ID NO: 75 - protein, 11 aa, synthetic construct
WGQGTQVTVSS

SEQ ID NO: 76 - protein, 11 aa, synthetic construct
WGQGTQVTVSS

SEQ ID NO: 77 - protein, 11 aa, synthetic construct
WGQGTQVTVSS

SEQ ID NO: 78 - protein, 25 aa, synthetic construct
  VARIANT 1: note = Q, E or D
  VARIANT 5: note = Q or V
  VARIANT 14: note = P or A
  VARIANT 19: note = R or K
  VARIANT 23: note = A, E, T or V
  VARIANT 25: note = S or A
XVQLXESGGGLVQXGGSLXLSCXAX

SEQ ID NO: 79 - protein, 14 aa, synthetic construct
  VARIANT 2: note = L, F or Y
  VARIANT 5: note = A or I
  VARIANT 9: note = G, E, or Q
  VARIANT 10: note = R or I
  VARIANT 12: note = G, F or W
  VARIANT 14: note = S or A
WXRQXPGKXXEXVX

SEQ ID NO: 80 - protein, 39 aa, synthetic construct
  VARIANT 5: note = V or A
  VARIANT 7: note = G or D
  VARIANT 10: note = S, T or A
  VARIANT 11: note = I or V
  VARIANT 15: note = D or N
  VARIANT 16: note = A, T or S
  VARIANT 23: note = R, Q or Y
  VARIANT 28: note = K, Q or R
  VARIANT 29: note = P or T
  VARIANT 34: note = V or I
  VARIANT 35: note = Y or V
  VARIANT 38: note = K, A or Y
  VARIANT 39: note = A, R or C
  VARIANT 2: note = A, S, H or D
  VARIANT 21: note = Y or N
  VARIANT 20: note = L, I or V
YXDSXKXRFXXSRDXXKNTXXLXMNSLXXEDTAXXYCXX

SEQ ID NO: 81 - protein, 11 aa, synthetic construct
  VARIANT 6: note = Q or L
  VARIANT 1: note = W or R
XGQGTXVTVSS

SEQ ID NO: 82 - protein, 262 aa, synthetic construct
EVQLVESGGGLVQAGGSLRLSCAASGRTFSSYAMGWFRQAPGKEREFVVAINWASGSTYYADSVKGRFTISRDNAKNTMYLQMNSLKPEDTAVYYCAAGYQINSGNYNFKDYEYDYWGQGTQVTVSSGSGGGGSGGGGSGGGGSGSQVQLQESGGGLVQPGGSLRLSCAASGIIFSDNRMDWYRQAPGKQREWVATLASYGWKTYADSVKGRFTISRDNTKNTVYLRMNSLKPEDTAVYYCKASSPVLNDIWGQGTQVTVSS

SEQ ID NO: 83 - protein, 402 aa, synthetic construct
EVQLVESGGGLVQAGGSLRLSCAASGRTFSSYAMGWFRQAPGKEREFVVAINWASGSTYYADSVKGRFTISRDNAKNTMYLQMNSLKPEDTAVYYCAAGYQINSGNYNFKDYEYDYWGQGTQVTVSSGGGGSGGGGSGGGGSEVQLVESGGGLVQAGGSLRLSCAASGRTFSSYAMGWFRQAPGKEREFVVAINWASGSTYYADSVKGRFTISRDNAKNTMYLQMNSLKPEDTAVYYCAAGYQINSGNYNFKDYEYDYWGQGTQVTVSSGSGGGGSGGGGSGGGGSQVQLQESGGGLVQPGGSLRLSCAASGIIFSDNRMDWYRQAPGKQREWVATLASYGWKTYADSVKGRFTISRDNTKNTVYLRMNSLKPEDTAVYYCKASSPVLNDIWGQGTQVTVSS

SEQ ID NO: 84 - protein, 288 aa, synthetic construct
EVQLVESGGGLVQAGGSLRLSCAASGRTFSSYAMGWFRQAPGKEREFVVAINWASGSTYYADSVKGRFTISRDNAKNTMYLQMNSLKPEDTAVYYCAAGYQINSGNYNFKDYEYDYWGQGTQVTVSSGGGGSGGGGSGGGGSGGGGSGGGGSGGGGSGGGGSGGGGSGGGGSQVQLQESGGGLVQPGGSLRLSCAASGIIFSDNRMDWYRQAPGKQREWVATLASYGWKTYADSVKGRFTISRDNTKNTVYLRMNSLKPEDTAVYYCKASSPVLNDIWGQGTQVTVSS

SEQ ID NO: 85 - protein, 295 aa, synthetic construct
EVQLVESGGGLVQAGGSLRLSCAASGRTFSSYAMGWFRQAPGKEREFVVAINWASGSTYYADSVKGRFTISRDNAKNTMYLQMNSLKPEDTAVYYCAAGYQINSGNYNFKDYEYDYWGQGTQVTVSSGGGGSGGGGSGGGGSGGGGSGGGGSGGGGSGGGGSGGGGSGGGGSQVQLQESGGGLVQAGGSLRLSCEASGRTFNTYNWGWFRQAPGKEREFVAAIRWSSSKTSYADSVKGRFTISRDNAKNTIYLQMNSLKPEDTAVYYCAASIVDFTTNPSHFGSWGQGTQVTVSS

SEQ ID NO: 86 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 575 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSDVQLVESGGGLVQPGGSLRLSCAASGIIFSDNRMDWYRQAPGKQREWVATLASYGWKTYADSVKGRFTISRDNAKNTVYLQMNSLRPEDTAVYYCKASSPVLNDIWGQGTLVTVSS

SEQ ID NO: 89 - protein, 582 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSDVQLVESGGGLVQPGGSLRLSCAASGRTFNTYNWGWFRQAPGKEREFVAAIRWSSSKTSYADSVKGRFTISRDNAKNTIYLQMNSLRPEDTAVYYCAASIVDFTTNPSHFGSWGQGTLVTVSS

SEQ ID NO: 90 - protein, 115 aa, synthetic construct
DVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 91 - protein, 248 aa, synthetic construct
DVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGGGGSGGGGSGGGGSQVQLQESGGGLVQPGGSLRLSCAASGIIFSDNRMDWYRQAPGKQREWVATLASYGWKTYADSVKGRFTISRDNTKNTVYLRMNSLKPEDTAVYYCKASSPVLNDIWGQGTQVTVSS

SEQ ID NO: 92 - protein, 255 aa, synthetic construct
DVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGGGGSGGGGSGGGGSQVQLQESGGGLVQAGGSLRLSCEASGRTFNTYNWGWFRQAPGKEREFVAAIRWSSSKTSYADSVKGRFTISRDNAKNTIYLQMNSLKPEDTAVYYCAASIVDFTTNPSHFGSWGQGTQVTVSS

SEQ ID NO: 93 - protein, 252 aa, synthetic construct
DVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGGGGSGGGGSGGGGSQVQLQESGGGLVQPGGSLRLSCVASGFTFDRYWMNWLRQIPGKEIEWVSTINTGGTGTYYDDSVKGRFSISRDDAKNTLYLTMNSLKTEDTAVYYCARGATYYRGNSAIRGQGTQVTVSS

SEQ ID NO: 94 - protein, 266 aa, synthetic construct
DVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGGGGSGGGGSGGGGSQVQLQESGGGLVQAGGSLKLSCAAAGRTFSRLAMGWFRQAPGKEREFVAAISENGDSIHYSDSVKGRFAVSRDNAKNTVYLQMNSLKPEDTAIYYCAADRAAYYCSGSGCYPSRAPAAASYDYWGQGTQVTVSS

SEQ ID NO: 95 - protein, 243 aa, synthetic construct
DVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGGGGSGGGGSGGGGSQVQLQESGGGLVQPGGSLRLSCAASGIIFSDNRMDWYRQAPGKQREWVATLASYGWKTYADSVKDRFTISRDNAKNTVNLYMNSLQPEDTAVYYCNSGQYWGQGTQVTVSS

SEQ ID NO: 96 - protein, 260 aa, synthetic construct
DVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGSGGGGSGGGGSGGGGSQVQLQESGGGLVQPGGSLRLSCTASGFTWDSYVIGWFRQAPGKGREGVSCLDVDDGSIYYHDSAKGRFSISRDNAKNTVYLQMNSLKPEDTAVYYCAAVNRASMRFRRCLQVLRYDYWGQGTQVTVSS

SEQ ID NO: 97 - protein, 2483 aa, Mus musculus
MRAVQLGPVPSGPRVALLPPLLLLLLLAAAGSAQAQAVDLDALCSYTWEAVDSKNNAVYKINVCGNVGISSCGPTSAICMCDLKTENCRSVGDSLLRSSARSLLEFNTTMGCQPSDSQHRIQTSITFLCGKTLGTPEFVTATDCVHYFEWRTTAACKKDIFKADKEVPCYAFDDKLQKHDLNPLIKLNGGYLVDDSDPDTSLFINVCRDIDSLRDPSTQLRVCPAGTAACLLKGNQAFDVGRPKEGLKLLSKDRLVLTYVKEEGEKPDFCNGHSPAVTVTFVCPSERREGTIPKLTAKSNCRYEVEWITEYACHRDYLQSESCSLSSEQHDITIDLSPLAQYGGSPYVSDGREYTFFINVCGDTKVSLCNNKEAAVCQEKKADSTQVKIAGRHQNQTLRYSDGDLTLIYSGGDECSSGFQRMSVINFECNKTAGKDGRGEPVFTGEVDCTYFFTWDTKYACIKEKEDLLCGAINGKKRYDLSVLARHSESEQNWEAVDGSQAESEKYFFINVCHRVLQEGKARNCPEDAAVCAVDKNGSKNLGKFVSSPTKEKGHIQLSYTDGDDCGSDKKISTNITLVCKPGDLESAPVLRAARSDGCFYEFEWHTAAACVLSKTEGENCTVLDAQAGFSFDLSLLTKKNGAYKVETEKYDFYINVCGPVSMDPCQSNSGACQVAKSGKSWNLGLSSTKLTYYDGMIQLSYRNGTPYNNEKHTPRATLITFLCDRDAGVGFPEYQEEDNSTYNFRWYTSYACPEEPLECMVTDPSMMEQYDLSSLVKSEGGSGGNWYAMENSREHVTRRKYYLNVCRPLNPVPGCDRYASACQMKYENHEGSLAETVSISNLGVAKIGPVVEESGSLLLEYVNGSACTTSDGQLTTYSTRIHLVCGRGFMNSHPIFTFNWECVVSFLWNTEAACPIQTITETDQACSIRDPSSGFVFNLSPLNDSAQGHVVLGIGKTFVFNICGAMPACGTVAGKPAYGCEAETQIEDIKDLRPQRPVGMERSLQLSAEGFLTLTYKGSSPSDRGTAFIIRFICNDDIYPGAPKFLHQDIDSTRGIRNTYFEFETALACTPSLVDCQVTDPAGNEYDLSALSMVRKPWTAVDTSAYGKRRHFYLSVCNPLPYIPGCHGIALGSCMVSEDNSFNLGVVQISPQATGNGSLSILYVNGDRCGDQRFSTRIVFECAQTSGSPMFQFVNNCEYVFVWRTVEACPVIREEGDNCQVKDPRHGNLYDLKPLGLNDTIVSVGEYTYYLRVCGKLSSDVCSAHDGSKAVSSCQEKKGPQGFQKVAGLLSQKLTFENGLLKMNYTGGDTCHKVYQRSTTIYFYCDRTTQKPVFLKETSDCSYMFEWRTQYACPPFNVTECSVQDAAGNSIDLSSLSRYSDNWEAVTRTGATEHYLINVCKSLSPHAGTEPCPPEAAVCLLNGSKPVNLGKVRDGPQWTDGVTVLQYVDGDLCPDKIRRRSTIIRFTCSDNQVNSRPLFISAVQDCEYTFSWPTPSACPVKSNTHDDCQVTNPSTGHLFDLSSLSGRAGINASYSEKGLVFMSICEENENCGPGVGACFGQTRISVGKASKRLSYKDQVLQLVYENGSPCPSLSDLRYKSVISFVCRPEAGPTNRPMLISLDKQTCTLFFSWHTPLACEQATECTVRNGSSIIDLSPLIHRTGGYEAYDESEDDTSDTTPDFYINICQPLNPMHGVPCPAGASVCKVPVDGPPIDIGRVTGPPIFNPVANEVYLNFESSTHCLADRYMNYTSLITFHCKRGVSMGTPKLIRTNDCDFVFEWETPIVCPDEVKTQGCAVTDEQLLYSFNLTSLSTSTFKVTRDARTYSIGVCTAAAGLGQEGCKDGGVCLLSGNKGASFGRLASMQLDYRHQDEAVILSYVNGDPCPPETDDGEPCVFPFIYKGKSYDECVLEGRAKLWCSKTANYDRDHEWGFCRQTNSYRMSAIIFTCDESEDIGRPQVFSEDRGCEVTFEWKTKVVCPPKKMECKFVQKHKTYDLRLLSSLTGSWDFVHEGNSYFINLCQRVYKGPLDCSERASICKKSATGQVQVLGLVHTQKLEVIDETVIVTYSKGYPCGGNKTASSVIELTCAKTVGRPAFKRFDSVSCTYYFYWYSRAACAVRPQEVTMVNGTLTNPVTGKSFSLGEIYFKLFSASGDMRTNGDNYLYEIQLSSITSSSYPACAGANICQVKPNDQHFSRKVGTSDMTKYYVQDGDLDVVFTSSSKCGKDKTKSVSSTIFFHCDPLVKDGIPEFSHETADCQYLFSWYTSAVCPLGVDFEDESAGPEYKGLSERSQAVGAVLSLLLVALTGCLLALLLHKKERRETVINKLTSCCRRSSGVSYKYSKVSKEEETDENETEWLMEEIQVPAPRLGKDGQENGHITTKAVKAEALSSLHGDDQDSEDEVLTVPEVKVHSGRGAEVESSQPLRNPQRKVLKEREGERLGLVRGEKARKGKFRPGQRKPTAPAKLVSFHDDSDEDLLHI

SEQ ID NO: 98 - protein, 2499 aa, Bos taurus
MEAAAGRSSHLGPAPAGRPPRCPLLLQLQLLLLLLLLPPGWVPGAAGTQGAEFPELCSYTWEAVDTKNNMLYKINICGNMGVAQCGPSSAVCMHDLKTDSFHSVGDSLLKTASRSLLEFNTTVNCKQQNHKIQSSITFLCGKTLGTPEFVTATDCVHYFEWRTTAACKKNIFKANKEVPCYAFDRELKKHDLNPLIKTSGAYLVDDSDPDTSLFINVCRDIEVLRASSPQVRVCPTGAAACLVRGDRAFDVGRPQEGLKLVSNDRLVLSYVKEGAGQPDFCDGHSPAVTITFVCPSERREGTIPKLTAKSNCRFEIEWVTEYACHRDYLESRSCSLSSAQHDVAVDLQPLSRVEASDSLFYTSEADEYTYYLSICGGSQAPICNKKDAAVCQVKKADSTQVKVAGRPQNLTLRYSDGDLTLIYFGGEECSSGFQRMSVINFECNQTAGNNGRGAPVFTGEVDCTYFFTWDTKYACVHEKEALLCGVSDGKQRFDLSALARHSELEQNWEAVDGSQREAEKKHFFINICHRVLQTGQARGCPEDAAVCAVDKNGSKNLGRFISSPTREKGNIQLSYSDGDECGGGQKIITNITLMCKPGDLESAPVLTTSRADGCFYEFEWRTAAACVLSRTEGDNCTVFDSQAGFSFDLTPLTKKDAYKVETDKYEFHINVCGPVSVGACPPDSGACQVSRSDRKSWNLGRSNAKLSYYDGMIQLTYRDGTPYNNEKRTPRATLITFLCDRDAGVGFPEYQEEDNSTYNFRWYTSYACPEEPLECIVTDPVTLDQYDLSRLAKSEGGPGGNWYSLDNGGARSTWRKYYINVCRPLNPVPGCDRYASACQMKYQGEQGSYSETVSISNLGVAKTGPMVEDSGSLLLEYVNGSACTTSDQRRTTYTTRIHLVCSTGSLYTHPIFSLNWECVVSFLWNTAAACPIRITTDIDQVCSIKDPNSGYVFDLNPLNNSRGYVVLGIGKTFLFNVCGDMPACGTLDGKPASGCEAEVQMDDMKTLKPGRLVGLEKSLQLSTEGFITLNYTGLPSHPNGRADAFIIRFVCNDDVYPGTPKFLHQDIDSSLGIRDTFFEFETALACVPSPVDCQVTDPAGNEYDLSGLSKARKPWTAVDTFDEGKKRTFYLSVCTPLPYIPGCHGTAVGCCLVTEDSKLNLGVVQISPQVGANGSLSLVYVNGDKCKNQRFSTRINLECAHTTGSPTFQLQNDCEYVFLWRTVEACPVVRAEGDYCEVRDPRHGNLYNLIPLGLNDTVVRAGEYTYYFRVCGELTSGVCPTSDKSKVISSCQEKRGPQGFQKVAGLFNQKLTYENGVLKMNYTGGDTCHKVYQRSTTIFFYCDRSTQAPVFLQETSDCSYLFEWRTQYACPPYDLTECSFKNEAGETYDLSSLSRYSDNWEAVTGTGSTEHYLINVCKSLSPQAGSDPCPPEAAVCLLGGPKPVNLGRVRDSPQWSQGLTLLKYVDGDLCPDQIRKKSTTIRFTCSESHVNSRPMFISAVEDCEYTFSWPTAAACAVKSNVHDDCQVTNPATGHLFDLSSLSGRAGFTAAYSEKGLVYLSVCGDNENCANGVGACFGQTRISVGKASKRLTYVDQVLQLVYEGGSPCPSKTGLSYKSVISFVCRPEVGPTNRPMLISLDKRTCTLFFSWHTPLACEQTTECSVRNGSSLIDLSPLIHRTGGYEAYDESEDDGSDTSPDFYINICQPLNPMHGLACPAGTAVCKVPVDGPPIDIGRVAGPPILNPIANEVYLNFESSTPCLADRHFNYTSLITFHCKRGVSMGTPKLLRTSVCDFVFEWETPLVCPDEVKTDGCSLTDEQLYYSFNLSSLSKSTFKVTRGPHTYSVGVCTAAAGLDEGGCKDGAVCLLSGSKGASFGRLASMKLDYRHQDEAVILSYANGDTCPPETEDGEPCVFPFVFNGKSYEECVVESRARLWCATTANYDRDHEWGFCKHSTSHRTSVIIFKCDEDADVGRPQVFSEVRGCEVTFEWKTKVVCPPKKMECKFVQKHRTYDLRLLSSLTGSWSFVHNGASYYINLCQKIYKGPQDCSERASVCKKSTSGEVQVLGLVHTQKLDVVDDRVIVTYSKGHYCGDNKTASAVIELTCAKTVGRPSFTRFDVDSCTYHFSWDSRAACAVKPQEVQMVNGTITNPANGRSFSLGDIYFKRFSASGDVRTNGDRYIYEIQLSSITGSSSPACSGASICQRKANDQHFSRKVGTSNQTRYYVQDGDLDVVFTSSSKCGKDKTKSVSSTIFFHCDPLVKDGIPEFSHETADCQYLFSWHTSAVCPLGAGFDEEIAGDDAQEHKGLSERSQAVGAVLSLLLVALTACLLTLLLYKKERREMVMSRLTNCCRRSANVSYKYSKVNKEEEADENETEWLMEEIQPPAPRPGKEGQENGHVAAKSVRAADTLSALHGDEQDSEDEVLTLPEVKVRPPGRAPGAEGGPPLRPLPRKAPPPLRADDRVGLVRGEPARRGRPRAAATPISTFHDDSDEDLLHV